(19) United States
(12) Patent Application Publication
(10) Pub. No.: US 2020/0056174 A1
(43) Pub. Date: Feb. 20, 2020

(54) COMPOSITIONS AND METHODS OF TREATING CANCER

(71) Applicant: DANA-FARBER CANCER INSTITUTE, INC., Boston, MA (US)

(72) Inventor: Donald Kufe, Wellesley, MA (US)

(21) Appl. No.: 16/330,927

(22) PCT Filed: Sep. 8, 2017
(86) PCT No.: PCT/US2017/050721

§ 371 Date: Mar. 6, 2019

Related U.S. Application Data
(60) Provisional application No. 62/384,950, filed on Sep. 8, 2016.

(51) Int. Cl. C12N 15/113; A61K 45/06

(52) U.S. Cl. CPC C12N 15/113; C12N 2310/14; A61K 45/06

(57) ABSTRACT

The present invention provides methods of treating cancer by de-repressing the anti-tumor immune response.

### RELATED APPLICATIONS

This application claims priority to, and the benefit of, U.S. Provisional Application No. 62/384,950, filed on Sep. 8, 2016, the contents of which is incorporated herein by reference in its entirety.

### GOVERNMENT INTEREST

This invention was made with government support under CA97098 and CA1664480 awarded by the National Cancer Institute. The government has certain rights in the invention.

### FIELD OF THE INVENTION

The present invention relates generally to de-repressing anti-tumor immunity.

### BACKGROUND OF THE INVENTION

A hallmark of human cancers is the evasion of immune destruction. Cancers are often infiltrated with immune cells that are ineffective in recognizing tumor antigens. Notably, however, the presence of immune cell infiltrates in “hot” tumors is associated with improved responsiveness to immunotherapeutic approaches, emphasizing the importance of reprogramming both “hot” and “cold” tumor microenvironments. In this way, immunotherapy has recently changed the landscape of NSCLC treatment. Blockade of the programmed death 1 (PD-1)/programmed death ligand 1 (PD-L1) immune checkpoint, in particular, is broadly effective in the treatment of NSCLCs and can extend survival in patients with tumors not responsive to targeted therapy. However, PD-1/PD-L1 blockade is associated with a response rate of about 20% in NSCLC and these responses are often of short duration. Thus, a need exists for composition and methods for increasing the efficacy of immunotherapy.

### SUMMARY OF THE INVENTION

In various aspects the invention provides methods of de-repressing an anti-tumor immune response in a subject having cancer comprising administering to the subject a MUC1 inhibitor, a MYC inhibitor, a TAK1 inhibitor, an NF-κB p65 pathway inhibitor, an IKK inhibitor, or a ZEB1 pathway inhibitor. The immune response is an innate immune response or an adaptive immune response. Optionally, the methods further include administering to the subject an immunotherapy.

In other aspects the invention provides methods of increasing the efficacy of an immunotherapy regimen by administering to the subject who has received or will receive an immunotherapy a MUC1 inhibitor, a MYC inhibitor, a TAK1 inhibitor, an NF-κB p65 pathway inhibitor, an IKK inhibitor, or a ZEB1 pathway inhibitor.

The immunotherapy is therapeutic antibody, a CAR T-cell therapy, a dendritic cell/tumor fusion, or a tumor vaccine.

The inhibitor is administered in an amount sufficient to decrease tumor PD-L1 transcription and/or TLR7 transcription. Alternatively, the inhibitor is administered in an amount sufficient to increase TLR9, IFNγ, MCP-1 or GM-CSF expression.

Optionally, the methods of the invention further include administering to the subject one or more checkpoint inhibitors. The checkpoint inhibitor is PD-1, PD-L1, PD-L2, CTLA-4, LAG-3, B7-H3, B7-H4, Tim3, BTLA, KIR, A2aR, and/or CD200.

In a further aspect, the invention provides method of augmenting the presentation of tumor associated antigen by a tumor by administering to said subject a MUC1 inhibitor, a MYC inhibitor, a TAK1 inhibitor, an NF-κB p65 pathway inhibitor, an IKK inhibitor, or a ZEB1 pathway inhibitor. The inhibitor is administered in an amount sufficient to increase the expression of TAP-1, TAP-2, MHC or Tapasin.

Unless otherwise defined, all technical and scientific terms used herein have the same meaning as commonly understood by one of ordinary skill in the art to which this invention pertains. Although methods and materials similar or equivalent to those described herein can be used in the practice of the present invention, suitable methods and materials are described below. All publications, patent applications, patents, and other references mentioned herein are expressly incorporated by reference in their entirety. In cases of conflict, the present specification, including definitions, will control. In addition, the materials, methods, and examples described herein are illustrative only and are not intended to be limiting.

Other features and advantages of the invention will be apparent from and encompassed by the following detailed description and claims.

### BRIEF DESCRIPTION OF THE DRAWINGS

FIG. 1. MUC1-C drives PD-L1 expression in NSCLC cells. A-C. H1975 (A), H460 (B) and A549 (C) NSCLC cells stably expressing a Control shRNA (CshRNA) or a MUC1 shRNA (MUC1shRNA) were analyzed for PD-L1 mRNA levels by qRT-PCR (left). The results (mean±SEM of three biological replicates each performed in triplicate) are expressed as relative mRNA levels compared to that obtained with cells expressing CshRNA (assigned a value of 1). Lysates were immunoblotted with the indicated antibodies (right). (D). A549 lung cancer cells were transfected to stably express an inducible control shRNA (left) or MUC1 shRNA (right). After treatment with doxycycline (DOX) for 72 h, lysates from the indicated cells were immunoblotted with antibodies against MUC1-C, PD-L1 and β-actin. E and F. H1975 (E) and H460 (F) cells were transiently transfected to express an empty vector or MUC1-C for 72 h.MUC1 (left) and PD-L1 (right) mRNA levels were determined by qRT-PCR. The results (mean±SEM of three biological replicates each performed in triplicate) are expressed as relative mRNA levels as compared to that obtained for cells expressing the empty vector (assigned a value of 1).

FIG. 2. Targeting the MUC1-C cytoplasmic domain downregulates PD-L1 expression. (A). Schematic representation of the MUC1-C subunit with the 58 aa extracellular domain (ED), the 28 aa transmembrane domain (TM), and the sequence of the 72 aa cytoplasmic domain (CD). The MUC1-C cytoplasmic domain contains a CQC motif that is necessary and sufficient for MUC1-C homodimerization and oncogenic function. GO-203 is a cell-penetrating peptide that targets the CQC motif and blocks MUC1-C homodimerization. GO-203 has been encapsulated into nanoparticles (GO-203/NPs) for delivery in mouse tumor models. The MUC1-C cytoplasmic domain binds directly to IKKβ, IKKγ, and NF-κB p65 and promotes the activation of NF-κB target genes. B and C. H1975 (B) and H460 (C) cells were infected with lentiviral vectors to stably express an empty vector or MUC1-C (AQA). The indicated cells were analyzed for PD-L1 mRNA levels by qRT-PCR. The results (mean±SEM of three determinations) are expressed as relative PD-L1 mRNA levels as compared to that obtained for the vector cells (assigned a value of 1). D and E. H1975 (D) and H460 (E) cells were treated with empty NPs or 2.5 μM GO-203/NPs at 0 and 72 h, and then harvested at 144 h. PD-L1 mRNA levels were determined by qRT-PCR. The results (mean±SEM of three determinations) are expressed as relative PD-L1 mRNA levels as compared to that obtained for the empty NP-treated cells (assigned a value of 1). (F). Mice bearing established H460 tumor xenografts (˜150 mm3) were treated weekly with intraperitoneal injections of empty NPs (squares) or 15 mg/kg GO-203/NPs (circles). The results are expressed as tumor volume (mean±SEM, 6 mice per group). * denotes p<0.05. ** denotes p<0.01. (G). Tumors obtained on day 14 were analyzed for PD-L1 mRNA levels by qRT-PCR (left). The results (mean±SEM of three biological replicates each performed in triplicate) are expressed as relative PD-L1 mRNA levels as compared to that obtained for the tumors obtained in control mice (assigned a value of 1). Tumor lysates from empty NP- and GO-203/NP-treated mice (day 14) were immunoblotted with the indicated antibodies (right).

FIG. 3. MUC1-C drives PD-L1 transcription by an NF-κB p65-dependent mechanism. (A). Schema of the pPD-L1-Luc reporter with positioning of the putative the NF-κB binding site at −377 to −387 upstream to the transcription start site. B and C. The indicated H1975 (B) and H460 (C) cells were transfected with the pPD-L1-Luc reporter for 48 h and then assayed for luciferase activity. The results are expressed as the relative luciferase activity (mean±SEM of three determinations) compared with that obtained from cells expressing the CshRNA (assigned a value of 1). D and E. H1975 (D) and H460 (E) cells were treated with 5 μM BAY-11-7085 or DMSO as the vehicle control for 18 h. PD-L1 mRNA levels were determined by qRT-PCR. The results (mean±SEM of three determinations) are expressed as relative PD-L1 mRNA levels as compared to that obtained for the control cells (assigned a value of 1). (F). H460 cells stably expressing a CshRNA or an NF-κB p65 shRNA (NF-κBshRNA) were transfected with the pPD-L1-Luc reporter for 48 h and then assayed for luciferase activity. The results are expressed as the relative luciferase activity (mean±SEM of three determinations) compared with that obtained from cells expressing the CshRNA (assigned a value of 1). (G). The indicated H460 cells were analyzed for PD-L1 mRNA levels by qRT-PCR (left). The results (mean±SEM of three biological replicates each performed in triplicate) are expressed as relative PD-L1 mRNA levels as compared to that obtained for cells expressing the CshRNA (assigned a value of 1). Lysates were immunoblotted with the indicated antibodies (right).

FIG. 4. MUC1-C/NF-κB p65 complexes occupy the PD-L1 promoter. (A). Soluble chromatin from H1975 cells was precipitated with anti-NF-κB or a control IgG. (B). In the re-ChIP experiments, NF-κB precipitates were released and then re-immunoprecipitated with an anti-MUC1-C. The final DNA samples were amplified by qPCR with primers for the PD-L1 promoter NF-κB binding region or GAPDH as a control. The results (mean±SEM of three determinations) are expressed as the relative fold enrichment compared to that obtained with the IgG control (assigned a value of 1). (C). Soluble chromatin from H1975/CshRNA and H1975/MUC1shRNA cells was precipitated with anti-NF-κB or a control IgG. The final DNA samples were amplified by qPCR with primers for the PD-L1 promoter NF-κB binding region or GAPDH as a control. The results (mean±SEM of three determinations) are expressed as the relative fold enrichment compared to that obtained for H1975/CshRNA cell chromatin (assigned a value of 1). (D). Soluble chromatin from H460 cells was precipitated with anti-NF-κB or a control IgG. (E). In re-ChIP experiments, NF-κB precipitates were released and then r-eimmunoprecipitated with an anti-MUC1-C. The final DNA samples were amplified by qPCR with primers for the PD-L1 promoter NF-κB binding region or as a control GAPDH. The results (mean±SEM of three determinations) are expressed as the relative fold enrichment compared with that obtained with the IgG control (assigned a value of 1). (F). Soluble chromatin from H460/CshRNA and H460/MUC1shRNA cells was precipitated with anti-NF-κB or a control IgG. The final DNA samples were amplified by qPCR with primers for the PD-L1 promoter NF-κB binding region or GAPDH as a control. The results (mean±SEM of three determinations) are expressed as the relative fold enrichment compared to that obtained for H460/CshRNA cell chromatin (assigned a value of 1).

FIG. 5. Targeting MUC1-C derepresses TLR9 expression. (A). Schema of the TLR9 promoter with positioning of the E-boxes upstream to the transcription start site. B and C. The indicated H1975 (B) and H460 (C) cells were analyzed for TLR9 mRNA levels by qRT-PCR. The results (mean±SEM of three determinations) are expressed as relative mRNA levels as compared to that obtained for the CshRNA cells (assigned a value of 1). (D). H460 cells stably express a control CshRNA or a ZEB1shRNA were analyzed for TLR9 mRNA levels by qRT-PCR. The results (mean±SEM of three determinations) are expressed as relative mRNA levels as compared to that obtained for the CshRNA cells (assigned a value of 1). (E). Soluble chromatin from H460 cells was precipitated with anti-ZEB1 or a control IgG. F. In re-ChIP experiments, ZEB1 precipitates were released and then re-immunoprecipitated with anti-MUC1-C. The final DNA samples were amplified by qPCR with primers for the TLR9 promoter ZEB1 binding region or as a control GAPDH. The results (mean±SEM of three determinations) are expressed as the relative fold enrichment compared to that obtained with the IgG control (assigned a value of 1). (G). Soluble chromatin from H460/CshRNA and H460/MUC1shRNA cells was precipitated with anti-ZEB1 or a control IgG. The final DNA samples were amplified by qPCR with primers for the TLR9 promoter ZEB1 binding region or as a control GAPDH. The results (mean±SEM of three determinations) are expressed as the relative fold enrichment compared to that obtained with H460/CshRNA cell chromatin (assigned a value of 1).

FIG. 6. Targeting MUC1-C activates IFN-γ expression. (A). Schema of the IFNG promoter with positioning of the E-boxes upstream to the transcription start site. B and C. The indicated H1975 (B) and H460 (C) cells were analyzed for IFN-γ mRNA levels by qRT-PCR. The results (mean±SEM of three determinations) are expressed as relative mRNA levels as compared to that obtained for the CshRNA cells (assigned a value of 1). (D). H460 cells stably express a control CshRNA or a ZEB1shRNA were analyzed for IFN-γ mRNA levels by qRT-PCR. The results (mean±SEM of three determinations) are expressed as relative mRNA levels as compared to that obtained for the CshRNA cells (assigned a value of 1). (E). Soluble chromatin from H460 cells was precipitated with anti-ZEB1 or a control IgG. (F). In the re-ChIP experiments, ZEB1 precipitates were released and then re-immunoprecipitated with anti-MUC1-C. The final DNA samples were amplified by qPCR with primers for the IFNG promoter ZEB1 binding region or as a control GAPDH. The results (mean±SEM of three determinations) are expressed as the relative fold enrichment compared to that obtained with the IgG control (assigned a value of 1). (G). Soluble chromatin from H460/CshRNA (left) and H460/MUC1shRNA was precipitated with anti-ZEB1 or a control IgG (right). The final DNA samples were amplified by qPCR with primers for the IFNG promoter ZEB1 binding region or as a control GAPDH. The results (mean±SD of three determinations) are expressed as the relative fold enrichment compared to that obtained with the CshRNA cells.

FIG. 7. (A). Targeting MUC1-C induces MCP-1 and GM-CSF expression by ZEB1-mediated mechanisms. A and B. The indicated H460 cells were analyzed for MCP-1 (A) and GM-CSF (B) mRNA levels by qRT-PCR. The results (mean±SEM of three determinations) are expressed as relative mRNA levels as compared to that obtained for the CshRNA cells (assigned a value of 1). (C). H460 tumors obtained on day 14 (see FIG. 2F) of treatment with empty NPs (open bars) or GO-203/NPs (solid bars) were analyzed for TLR9, IFN-γ, MCP-1 and GM-CSF mRNA levels by qRT-PCR. The results (mean±SEM of three biological replicates each performed in triplicate) are expressed as relative mRNA levels as compared to that obtained for the tumors obtained in empty NP-treated mice (assigned a value of 1). ** denotes p value<0.05. (D). Kaplan-Meier plot comparing the overall survival of patients with NSCLC. Patients were stratified with the high (red) or low (black) expression of TLR9, IFN-γ, MCP-1 and GM-CSF against the median average. The survival curves were compared using Log-rank (Mantel-Cox) test. H.R.: Hazard Ratio. (E). Proposed schema of a MUC1-C-induced proinflammatory program linking EMT (blue) and immune evasion (red) of NSCLC cells. MUC1-C activates the proinflammatory TAK1→IKK→NF-κB p65 pathway (32-34). MUC1-C upregulates TLR7, which also contributes to NF-κB p65 activation, survival and chemoresistance of NSCLC cells (40). MUC1-C forms a complex with NF-κB p65 and induces the activation of NF-κB target genes, including MUC1 itself, in an autoinductive circuit (33). MUC1-C also promotes occupancy of NF-κB p65 on the ZEB1 (44) and PD-L1 promoters and contributes to activation of these genes. The upregulation of ZEB1 and the formation of MUC1-C/ZEB1 complexes suppresses miR-200c and thereby induces EMT (44). Of note, PD-L1 is also a target of miR-200 (15), invoking the possibility that the MUC1-C→NF-κB p65→ZEB1 pathway could increase PD-L1 expression by both transcriptional and post-transcriptional mechanisms. Our results further support a role for MUC1-C/ZEB1 complexes in suppression of TLR9, IFNG, MCP-1 and GM-CSF, linking EMT with immune evasion. Thus, targeting MUC1-C suppresses PD-L1 and induces TLR9, IFN-γ, MCP-1 and GM-CSF expression, supporting the notion that MUC1-C is of importance for immune evasion.

FIG. 8. Silencing MUC1-C decreases activation of the pPD-L1 reporter. The indicated A549 cells were transfected with the pPD-L1-Luc reporter for 48 h and then assayed for luciferase activity. The results are expressed as the relative luciferase activity (mean±SEM of three biological replicates each performed in triplicate) compared with that obtained with cells expressing the CshRNA (assigned a value of 1).

FIG. 9. Overexpression of MUC1-C (AQA) suppresses the pPD-L1-Luc reporter. A and B. H1975 (A) and H460 (B) cells stably expressing an empty vector or MUC1-C (AQA) were transfected with the pPD-L1-Luc reporter for 48 h and then assayed for luciferase activity. The results are expressed as the relative luciferase activity (mean±SEM of three determinations) compared with that obtained from cells expressing the empty vector (assigned a value of 1).

FIG. 10. Silencing MUC1-C has little effect on PD-1, PD-L2, CTLA-4, TIM-3 and LAG-3 expression. A and B. The indicated H1975 (A) and H460 (B) cells were analyzed for PD-1, PD-L2, CTLA-4, TIM-3 and LAG-3 mRNA levels by qRT-PCR. The results (mean±SEM of three determinations) are expressed as relative mRNA levels as compared to that obtained for the CshRNA cells (assigned a value of 1).

FIG. 11. Targeting MUC1-C suppresses TLR7 expression by an NF-κB-dependent mechanism. (A). Schema of the TLR7 promoter with localization of the NF-κB binding site. (B). The indicated H1975 (left) and H460 (right) cells were analyzed for TLR7 mRNA levels by qRT-PCR. The results (mean±SEM of three determinations) are expressed as relative mRNA levels as compared to that obtained for the CshRNA cells (assigned a value of 1). (C). H1975 (left) and H460 (right) cells were treated with 5 μM BAY-11-7085 or DMSO as the vehicle control for 18 h. TLR7 mRNA levels were determined by qRT-PCR. The results (mean±SEM of three determinations) are expressed as relative TLR7 mRNA levels as compared to that obtained for the control cells (assigned a value of 1). (D). The indicated H460 cells were analyzed for TLR7 mRNA levels by qRT-PCR. The results (mean±SEM of three determinations) are expressed as relative TLR7 mRNA levels as compared to that obtained for cells expressing the CshRNA (assigned a value of 1).

FIG. 12. MUC1 inversely correlates with TLR9, IFN-γ and MCP-1 expression. A-C. Clinical dataset of NSCLC patients were downloaded from Gene Expression Omnibus (GEO) under the accession number GSE72094 (n=442). Log 2 expression values of MUC1-C (merck-NM_001018016at) were assessed for correlation with TLR9 (merck-NM_017442_at) (A), IFN-γ (merck-NM_000619_at) (B) and MCP-1 (merck-NM_002982_at) (C) (left). RNA sequencing data from the TCGA dataset (n=576) was normalized and the correlation between MUC1 and TLR9 (A), IFN-γ (B) and MCP-1 (C) was assessed by the Spearman's rank correlation coefficient (right). *** denotes p value <0.001. The shaded area represents the 95% confidence interval.

FIG. 13. MUC1-C regulates PD-L1 and IFN-γ expression in LLC NSCLC cells. (A) Lewis Lung Carcinoma (LLC) NSCLC cells stably expressing a control empty vector (LLC/Vector) or full length MUC1 (LLC/MUC1) were analyzed for MUC1, PD-L1 and IFN-γ mRNA levels by qRT-PCR. The results (mean±SEM of three biological replicates each performed in triplicate) are expressed as relative mRNA levels as compared to that obtained for the vector cells (assigned a value of 1). (B) Lysates from LLC/Vector and LLC/MUC1 cells were immunoblotted with the indicated antibodies. (C) LLC cells expressing a control empty vector (LLC/Vector) or MUC1-C (LLC/MUC1-C) were analyzed for MUC1, PD-L1 and IFN-γ mRNA levels by qRT-PCR. The results (mean±SEM of three biological replicates each performed in triplicate) are expressed as relative mRNA levels as compared to that obtained for the vector cells (assigned a value of 1. (D) LLC/MUC1 cells were treated with empty NPs or 2.5 μM GO-203/NPs for 72 h. Cells were analyzed for MUC1, PD-L1 and IFN-γ mRNA levels by qRT-PCR. The results (mean±SEM of three biological replicates each performed in triplicate) are expressed as relative mRNA levels as compared to that obtained for the NP-treated cells (assigned a value of 1). (E) Lysates from the designated LLC/MUC1 cells were immunoblotted with the indicated antibodies.

FIG. 14. Targeting MUC1-C activates the LLC tumor immune microenvironment in a MUC1.Tg mouse model. (A) Mice bearing established LLC/MUC1 tumor xenografts (˜150 mm3) were treated weekly with intraperitoneal injections of empty NPs (squares) or 15 mg/kg GO-203/NPs (triangles). The results are expressed as tumor volume (mean±SEM, 6 mice per group). * denotes p<0.05. (B) Tumors harvested from empty NP- and GO-203/NP-treated mice (day 10) were analyzed for MUC1, PD-L1 and IFN-γ mRNA levels by qRT-PCR. The results (mean±SEM of three biological replicates each performed in triplicate) are expressed as relative mRNA levels as compared to that obtained for the control NP-treated mice (assigned a value of 1). (C) Tumors obtained on day 10 were immunoblotted with the indicated antibodies. (D-F) Single cell suspensions were generated from the LLC/MUC1 tumor tissues and subjected to FACS analysis. (D) In a representative histogram, tumor cells from NP-treated (profile #1) and GO-203/NP-treated (profile #2) mice were analyzed for PD-L1 expression (left). An isotype identical antibody was used as an internal control (profile #3) (left). The percentage of PD-L1-positive tumor cells is expressed as the mean±SEM for 5 tumors per group (right). (E) Expression levels of Ki67 on T-cells relative to PD-L1 on tumor cells. (F) Tumor infiltrating CD8+ cells were analyzed for CD69 expression. The results are expressed as the percentage (mean±SEM for 5 tumors per group) of CD69 positive cells.

FIG. 15. Functional evaluation of TILs from LLC/MUC1 tumors. (A-E) Immune cells were isolated from LLC/MUC1 tumors and then stimulated ex vivo for 6 h. (A) The CD45+CD3+ tumor-infiltrating population was analyzed for CD8+ T-cells and CD4+Foxp3+ Tregs. The results are expressed as the CD8+/CD4+Foxp3 ratio (mean±SD for 4 tumors per group). (B) Representative histogram depicting IFN-γ production by CD8+ T-cells from NP-treated (profile #1) and GO-203/NP-treated (profile #2) LLC/MUC1 tumors (left). An isotype identical antibody was used as an internal control (profile #3) (left). The results are expressed as the percentage (mean±SEM for 5 tumors per group) of IFN-γ+ cells (right). (C) Representative histogram showing CD107α expression by CD8+ T-cells from NP-treated (profile #1) and GO 203/NP treated (profile #2) LLC/MUC1 tumors (left). The results are expressed as the mean fluorescent intensity (MFI; mean±SEM of 5 tumors per group) (right). (D) CD8+ T-cells were analysed for granzyme B secretion. The results are expressed as the percentage (mean±SEM for 5 tumors per group) of granzyme B positive cells. (E) Lymph nodes obtained from NP- and GO-203/NP-treated mice were incubated with LLC/MUC1 target cells at the indicated ratios. The results are expressed as percentage cytotoxicity (mean±SEM for 5 mice per group) comparing NP-treated mice (open bars) with GO-203/NP-treated mice (solid bars).

FIG. 16. Structure of the MUC1-C subunit. MUC1-C consists of a 58-aa extracellular, a 28-aa transmembrane, and a 72-aa cytoplasmic domain. Highlighted is the aa sequence of the intrinsically disordered cytoplasmic domain and interactions with the IKK→NF-κB p65 pathway. The CQC motif is necessary for MUC1-C homodimerization, nuclear import and oncogenic function. The CQC motif is the target of the GO-203 peptide, which blocks MUC1-C homodimerization.

FIG. 17. Overexpression of MUC1 in NSCLC negatively correlates with CD8, IFNG and granzyme B (GZMB). (A) Microarray data from Oncomine database are expressed as box plots (25th-75^{th} percentiles) for MUC1 expression in normal lung tissues (n=20) and lung adenocarcinoma (n=226). The data were log 2 transformed and median centered. (B-D) RNA sequencing data of lung cancer patients was obtained from cBioPortal TCGA data set. Correlations between MUC1 expression and that for CD8 (B), IFNG (C) and GZMB (D) were assessed using Spearman's rank correlation coefficient, where p<0.05 was considered as statistically significant.

FIG. 18. Expression of CD8 and IFNG in NSCLC correlates with survival. (A-B) Kaplan-Meier plot comparing the overall survival of patients with NSCLC in the TCGA data set. Patients were stratified with the high (red) or low (blue) expression of CD8 (A) and IFNG (B) against the median average. The survival curves were compared using log-rank (Mantel-Cox) test. HR, hazard ratio.

FIG. 19. MUC1-C induces PD-L1 expression. (A) Lysates from the designated basal A and basal B TNBC cells were immunoblotted with the indicated antibodies. (B-C) BT-549 cells were transduced to stably express a tetracycline-inducible MUC1 shRNA (tet-MUC1shRNA). Cells treated with or without 500 ng/ml DOX for 4 d were analyzed for MUC1 (left) and PD-L1 mRNA levels (right) by qRT-PCR. The results (mean±SD of 3 determinations) are expressed as relative mRNA levels compared with that obtained for control DOX-untreated cells (assigned a value of 1) (B). Lysates from cells treated with or without 500 ng/ml DOX for 7 d were immunoblotted with the indicated antibodies (C). (D-E) MDA-MB-231/tet-MUC1shRNA cells treated with or without 200 ng/ml DOX for 4 d were analyzed for MUC1 (left) and PD-L1 mRNA levels (right) by qRT-PCR (mean±SD of 3 determinations) (D). Lysates from cells treated with or without 200 ng/ml DOX for 7 d were immunoblotted with the antibodies (E).

FIG. 20. Effects of DOX treatment on BT-549/tet-CshRNA, BT-549/tet-MUC1 shRNA #2, MDA-MB-231/tet-CshRNA, and SUM-159/tet-MUC1 shRNA cells. (A) BT-549 cells were stably transduced a tetracycline-inducible control shRNA (tet-CshRNA). Cells treated with or without 500 ng/ml DOX for 4 d were analyzed for MUC1 (left) and PD-L1 (right) mRNA levels by qRT-PCR. The results (mean±SD of 3 determinations) are expressed as relative mRNA levels compared with that obtained for control DOX-untreated cells (assigned a value of 1). (B) BT-549/tet-MUC1shRNA cells were treated with and without 500 ng/ml DOX for 7 d. Cell number was determined by Alamar blue staining. The results (mean±SD of 6 determinations) are expressed as relative cell number compared with that obtained for control DOX-untreated cells (assigned a value of 1). (C-D) BT-549 cells were stably transduced tetracycline-inducible MUC1 shRNA #2 (tet-MUC1shRNA #2). Cells treated with or without 500 ng/ml DOX for 4 d were analyzed for MUC1 (left) and PD-L1 (right) mRNA levels by qRT-PCR. The results (mean±SD of 3 determinations) are expressed as relative mRNA levels compared with that obtained for control DOX-untreated cells (assigned a value of 1). (C). Lysates from cells treated with or without 500 ng/ml DOX for 7 d were immunoblotted with the indicated antibodies (D). (E). MDA-MB-231 cells were stably transduced a tetracycline-inducible control shRNA (tet-CshRNA). Cells treated with or without 200 ng/ml DOX for 4 d were analyzed for MUC1 (left) and PD-L1 (right) mRNA levels by qRTPCR. The results (mean±SD of 3 determinations) are expressed as relative mRNA levels compared with that obtained for control DOXuntreated cells (assigned a value of 1). (F-G). SUM-159 cells were stably transduced to express a tetracycline-inducible MUC1 shRNA. Cells treated with or without 200 ng/ml DOX for 5 d were analyzed for MUC1 (left) and PD-L1 (right) mRNA levels by qRT-PCR. The results (mean±SD of 3 determinations) are expressed as relative mRNA levels compared with that obtained for control DOX-untreated cells (assigned a value of 1) (F). Lysates from cells treated with or without 200 ng/ml DOX for 7 d were immunoblotted with the indicated antibodies (G).

FIG. 21. Targeting MUC1-C suppresses PD-L1 expression. (A) Schema of MUC1-C with the 58 amino acid (aa) extracellular domain (ED), the 28 aa transmembrane domain (TM), and the 72 aa cytoplasmic domain (CD). The CQC motif of the CD domain is indispensable for MUC1-C homodimerization, and is targeted by the cell-penetrating GO-203 peptide. Highlighted are interactions of the MUC1-C cytoplasmic domain with the NF κB p65 and MYC pathways. (B) BT-20 cells stably transduced to express a control or MUC1-C vector were analyzed for PDL1 mRNA levels by qRT-PCR. The results (mean±SD of 3 determinations) are expressed as relative PD-L1 mRNA levels compared to that obtained for vector cells (assigned a value of 1) (left). Lysates were immunoblotted with the indicated antibodies (right). (C) BT-549 cells were transfected to stably express an empty vector or MUC1-C (AQA) mutant. Lysates were immunoblotted with the indicated antibodies. (D-F). BT-549 (D), MDA-MB-231 (E), and BT-20/MUC1-C (F) cells treated with empty NPs or 2.5 μM GO-203/NPs for 5 d were analyzed for PD-L1 mRNA levels by qRT-PCR. The results (mean±SD of 3 determinations) are expressed as relative PD-L1 mRNA levels compared to that obtained for empty NPs (assigned a value of 1) (left). Lysates from cells treated with empty NPs or 2.5 μM GO-203/NPs for 7 d were immunoblotted with the indicated antibodies (right).

FIG. 22. MUC1-C→MYC signaling induces PD-L1 expression. (A-B). Lysates from BT-549/tet-MUCshRNA (A) and MDA-MB-231/tet-MUCshRNA (B) cells treated with or without DOX for 7 d were immunoblotted with the indicated antibodies. (C-D). Lysates from BT-549 (C) and MDA-MB-231 (D) cells treated with 5 μM CP-2 or 5 μM GO-203 for 3 d were immunoblotted with the indicated antibodies. (E-F). BT-549/tet-MYCshRNAcells treated with or without 200 ng/ml DOX for 1 d were analyzed for MYC and PD-L1 levels by qRT-PCR. The results (mean±SD of 3 determinations) are expressed as relative mRNA levels compared with that obtained for control DOX-untreated cells (assigned a value of 1) (E). Lysates from cells treated with or without 200 ng/ml DOX for 3 d were immunoblotted with the indicated antibodies (F). (G-H). MDA-MB-231/tet-MYCshRNA cells treated with or without 200 ng/ml DOX for 1 d were analyzed for MYC and PD-L1 levels by qRT-PCR (mean±SD of 3 determinations) (G). Lysates from cells treated with or without 200 ng/ml DOX for 3 d were immunoblotted with the indicated antibodies (H).

FIG. 23. Effects of treatment with the JQ1 BET bromodomain inhibitor. (A-B). BT-549 (A) and BT-20/MUC1-C (B) cells treated with 5 μM JQ1 or vehicle control for 48 h were analyzed for MYC and PD-L1 mRNA levels by qRT-PCR. The results (mean±SD of 3 determinations) are expressed as relative mRNA levels compared with that obtained for control cells (assigned a value of 1) (left). Cell lysates were immunoblotted with the indicated antibodies (right).

FIG. 24. MUC1-CR→NF-kB p65 signaling induces PD-L1 expression. (A) Lysates from BT-549/tet-MUC1shRNA cells treated with or without 200 ng/ml DOX for 7 d were immunoblotted with indicated antibodies. (B) Lysates from MDA-MB-231/tet-MUC1shRNA cells treated with or without 500 ng/ml DOX for 7 d were immunoblotted with the indicated antibodies. (C-D). BT-549 cells and MDA-MB-231 cells were stably transduced to express a control shRNA (CshRNA) or NF-kB p65 shRNA (p65shRNA). Cells were analyzed for PD-L1 levels by qRT-PCR. The results (mean±SD of 3 determinations) are expressed as relative PD-L1 mRNA levels compared to that obtained for CshRNA cells assigned a value of 1) (left). Lysates were immunoblotted with the indicated antibodies (right). (E-F) BT-549 (F) and BT-20/MUC1-C (G) cells treated with 5 μM BAY-11-7085 (BAY-11) or vehicle control for 24 h were analyzed for PD-L1 mRNA levels by qRT-PCR (mean±SD of 3 determinations) (left). Cell lysates were immunoblotted with the indicated antibodies (right).

FIG. 25. MUC1-C enhances MYC and NF-κB p65 occupancy on the PDL1 promoter. (A) Schema of the pPD-L1 promoter with highlighting of the E-box at −159 to −164 and NF-κB binding site at −378 to −387 upstream to the transcription start site (TSS). (B) BT-549/tet-MUC1shRNA cells cultured with or without DOX for 5 d were transfected with the pPD-L1-Luc reporter for 48 h and then assayed for luciferase activity. The results (mean±SD of 3 determinations) are expressed as the relative luciferase activity compared to that obtained for control DOX-untreated cells (assigned a value of 1). (C) BT-549 cells treated with NPs or GO-203/NPs for 4 d were transfected with pPD-L1-Luc reporter for 48 h and then assayed for luciferase activity. The results (mean±SD of 3 determinations) are expressed as the relative luciferase activity compared to that obtained with empty NP-treated cells (assigned a value of 1). (D) Soluble chromatin from BT-549/tet-MUC1shRNA cells was precipitated with anti-MYC or a control IgG (left). The final DNA samples were amplified by qPCR with primers for the PD-L1 promoter MYC binding region or GAPDH as a control. The results (mean±SD of three determinations) are expressed as the relative fold enrichment compared to that obtained with the IgG control (assigned a value of 1). Soluble chromatin from 549/tet-MUC1shRNA cells cultured with or without DOX for 5 d was precipitated with anti-MYC or a control IgG. The final DNA samples were amplified by qPCR. The results (mean±SEM of three determinations) are expressed as the relative fold enrichment compared to that obtained for control DOX-untreated cells (assigned a value of 1) (right). (E) Soluble chromatin from BT-549/tet-MUC1shRNA cells was precipitated with anti-NF-κB p65 or a control IgG (left). The final DNA samples were amplified by qPCR with primers for the PD-L1 promoter NF-κB binding region or GAPDH as a control. The results (mean±SD of three determinations) are expressed as the relative fold enrichment compared to that obtained with the IgG control (assigned a value of 1). Soluble chromatin from BT-549/tet-MUC1shRNA cells cultured with or without DOX for 5 d was precipitated with anti-NF-κB p65 or a control IgG (right). The final DNA samples were amplified by qPCR. The results (mean±SEM of three determinations) are expressed as the relative fold enrichment compared to that obtained for control DOXuntreated cell chromatin (assigned a value of 1). (F) Soluble chromatin from BT-549 and MDA-MB-468 cells was precipitated with anti-MUC1-C or a control IgG. The final DNA samples were amplified by qPCR with primers for the PD-L1 promoter or GAPDH as a control. The results (mean±SD of three determinations) are expressed as the relative fold enrichment compared to that obtained with the IgG controls (assigned a value of 1). *p<0.05. (G) Soluble chromatin from MDA-MB-468 cells was precipitated with anti-MYC (left), anti-NF-κB p65 (right) or a control IgG (left). The final DNA samples were amplified by qPCR with primers for the PD-L1 promoter or GAPDH as a control. The results (mean±SD of three determinations) are expressed as the relative fold enrichment compared to that obtained with the IgG controls (assigned a value of 1). # p>0.05.

FIG. 26. MUC1-C drives PD-L1 expression in mouse Eo771 TNBC cells. (A-B). Eo771 TNBC cells were stably transduced to express human MUC1-C (Eo771/MUC1-C). Cells were analyzed for PD-L1 levels by qRT-PCR. The results (mean±SD of 3 determinations) are expressed as relative PD-L1 mRNA levels compared to that obtained for Eo771/vector cells (assigned a value of 1) (A) Lysates were immunoblotted with the indicated antibodies (B). (C) Eo771/MUC1-C cells treated with 5 μM JQ1 or vehicle control for 48 h were analyzed for PD-L1 mRNA levels by qRT-PCR (mean±SD of 3 determinations) (left). Cell lysates were immunoblotted with the indicated antibodies (right). (D) Eo771/MUC1-C cells treated with 5 μM BAY-11-7085 (BAY-11) or vehicle control for 24 h were analyzed for PD-L1 mRNA levels by qRT-PCR (mean±SD of 3 determinations) (left). Cell lysates were immunoblotted with the indicated antibodies (right). (E) Eo771/MUC1-C cells treated with empty NPs or 2.5 μM GO-203/NPs for 5 d were analyzed for PD-L1 mRNA levels by qRT-PCR (mean±SD of 3 determinations) (left). Lysates from cells treated with empty NPs or 2.5 μM GO-203/NPs for 7 d were immunoblotted with the indicated antibodies (right).

FIG. 27. Targeting MUC1-C in Eo771/MUC1-C tumors activates the immune microenvironment. (A) Eo771/MUC1-C cells were injected subcutaneously into the flanks of MUC1.Tg mice. Left panel. Mice with established tumors of approximately 150 mm3 were pair-matched and then treated with empty NPs (diamonds) or 15 mg/kg GO-203/NPs (squares) (left). The results are expressed as tumor volume (mean±SEM; 5 mice per group). One of the tumors in the GO-203/NP-treated group was undetectable at the time of harvest. *p<0.05. Tumors were harvested on day 16 when the controls showed signs of necrosis. Right panel. In a subsequent experiment, mice were treated with PBS (diamonds) or 10 mg/kg anti-PD-L1 (squares) on days 0 and 5. The results are expressed as tumor volume (mean±SEM; 6 mice per group). Tumors in the control group showed signs of necrosis on day 16 when the study was terminated according to the animal protocol. (B) Tumor cells were analyzed for PD-L1 mRNA levels by qRT-PCR. The results (mean±SD of 4 determinations) are expressed as relative mRNA levels compared with that obtained for empty NP-treated tumors (assigned a value of 1) (left). Lysates were immunoblotted with the indicated antibodies (right). (C-E). Single cell suspensions were prepared for FACS analysis. (C) In a representative histogram, tumor cells from NPtreated (profile #1) and GO-203/NP-treated (profile #2) mice were analyzed for PD-L1 expression (left). An isotype identical antibody was used as a control (profile #3) (left). The percentage of PD-L1-positive tumor cells is expressed as the mean±SD (4 tumors per group) (right). (D) Tumor-infiltrating CD8+ T-cells were analyzed for CD69 and granzyme B expression. The results are expressed as the percentage (mean±SD; n=4) of CD69 (left) and granzyme B (right) positive cells. E Tumor-infiltrating immune cells were isolated by Ficoll separation and stimulated with the Leucocyte Activation Cocktail. CD8+ T-cells were analyzed for expression of the CD107α degranulation marker (left), IFN-γ (middle) and granzyme B (right). The results are expressed as the percentage (mean±SD; n=4) of positive cells. (F) Lymph nodes obtained from NP- and GO-203/NP-treated mice were disrupted into cell single suspensions. Effectors were plated in 96-well plates with Eo771/MUC1-C target cells at a 3:1 ratio. After 6 h, T-cell mediated cytotoxicity was assayed measuring LDH release. The results are expressed as percentage cytotoxicity (mean±SD; n=4).

FIG. 28. Targeting MUC1-C in E0771/MUC1-C tumors increases CD69 and granzyme B expression. (A-B). Single cell tumor suspensions were prepared for FACS analysis. In representative histograms, tumor cells from NP-treated (profile #1) and GO-203/NP-treated (profile #2) mice were analyzed for CD69 (A) and granzyme B (B) expression. An isotype identical antibody was used as a control (profile #3).

FIG. 29. Targeting MUC1-C in Eo771/MUC1-C tumors activates CD8+ T-cells. Tumor-infiltrating immune cells were isolated by Ficoll separation and stimulated with the Leucocyte Activation Cocktail. In representative histograms, CD8+ T cells from NP-treated (profile #1) and GO-203/NP-treated (profile #2) mice were analyzed for expression of the CD107α degranulation marker (A), IFN-γ (B) and granzyme B (C). An isotype identical antibody was used as a control (profile #3).

FIG. 30. Correlation between MUC1 and T-cell activation in TNBCs. (A-C). Gene expression data obtained from of TNBCs was obtained from GSE25066 datasets. Correlation between MUC1 and CD8A/B (A), CD69 (B) and GZMB (C) expression (C) were assessed using the Spearman's coefficient, where p<0.05 was considered as statistically significant. (D-F). Kaplan-Meier plots comparing the Relapse-Free Survival (RFS) of TNBC patients. Patients were stratified with high (red) or low (blue) expression of CD8 (D), CD69 (E) and GZMB (F) against the median. The survival curves were compared using the log-rank test. HR, hazard ratio.

FIG. 31. Proposed model for MUC1-C-induced integration of PD-L1 expression with EMT, CSC state and epigenetic programming in basal B TNBC cells. The present results demonstrate that MUC1-C activates the PD-L1 gene by NF-κB p65- and MYC-mediated mechanisms. MUC1-C→NF-κB p65 signaling also activates the ZEB1 gene and thereby represses miR-200c with induction of the EMT program and CSC state (44,34). Additionally, the MUC1-C→NF-κB p65 pathway promotes epigenetic reprogramming by induction of genes encoding DNMT1/3b and components of the PRC2 complex, including EZH2 (96,108). Moreover, MUC1-C-induced activation of the MYC pathway induces BMI1 expression and PRC1-mediated epigenetic alterations (98). In this way, MUC1-C integrates PD-L1 expression with the EMT program, CSC state and epigenetic reprogramming in basal B TNBC cells.

### DETAILED DESCRIPTION OF THE INVENTION

The immune system plays a critical role in protecting the host from cancer. Notably, the tumor microenvironment is an important aspect of cancer biology that contributes to tumor initiation, tumor progression, and responses to therapy. Cells and molecules of the immune system are a fundamental component of the tumor microenvironment.

Harnessing the inherent ability of the immune system to eliminate tumor cells represents the most promising anti-cancer strategy since the development of chemotherapy, however, in most cases, the optimal anti-tumor response is drastically reduced because of the tumor's ability to evade immune destruction. Cancers are often infiltrated with immune cells that are ineffective in recognizing tumor antigens.

Immunotherapy has recently changed the landscape of cancer treatment. For example, blockade of the programmed death 1 (PD-1)/programmed death ligand 1 (PD-L1) immune checkpoint, is broadly effective in the treatment of NSCLCs and can extend survival in patients with tumors not responsive to targeted therapy. However, PD-1/PD-L1 blockade is associated with a response rate of about 20% in NSCLC and these responses are often of short duration. These findings support the premise that evasion of immune recognition and destruction contributes to the pathogenesis of cancer and that additional approaches are needed to enhance the effectiveness of immunotherapy.

Studies in genetically engineered mouse models (GEMMs) have demonstrated that NSCLCs driven by mutant EGFR activate the PD-1/PD-L1 pathway and thereby suppress T-cell function. Similarly KRAS-driven NSCLCs also increase inflammatory cytokine production to suppress T-cell activity in the tumor microenvironment.

In addition, (PD-L1 is upregulated in triple-negative breast cancer (TNBC) and is of importance to the pathogenesis of this refractory disease. Mucin 1 (MUC1) is also overexpressed in TNBC cells and confers a poor prognosis. Our studies provide insights into the involvement of MUC1-C in immune evasion of TNBCs and support the targeting of MUC1-C as a potential immunotherapeutic approach for the treatment of patients with TNB.

The present invention sought to identify the mechanisms by which tumor cells induce PD-L1 expression and immunosuppressive cytokine production in order to develop more effective immunotherapeutic approaches.

Mucin 1 (MUC1) is a transmembrane glycoprotein that is aberrantly overexpressed in >80% of NSCLCs (16). The overexpression of MUC1 in NSCLCs is associated with poor disease-free and overall survival, emphasizing the potential importance of MUC1 to NSCLC pathogenesis. MUC1 consists of two subunits: an N-terminal extracellular mucin subunit (MUC1-N) and a transmembrane C-terminal subunit (MUC1-C) that functions as an oncoprotein (22, 23). MUC1-C includes a 58-amino acid extracellular domain, which forms complexes with galectin-3 and thereby cell surface receptor tyrosine kinases, such as EGFR (24). The MUC1-C 72-amino acid cytoplasmic domain is an intrinsically disordered structure (25), which has the plasticity to interact with multiple kinases and effectors that have been linked to transformation (22, 23). In this context, the MUC1-C cytoplasmic domain activates the PI3K→AKT and MEK→ERK pathways in NSCLC and other carcinoma cells (26-28). The MUC1-C cytoplasmic domain also binds directly to certain transcription factors, such as β-catenin/TCF4 and STAT1/3, and promotes activation of their target genes (29-31). In addition, MUC1-C directly activates the TAK1→IKK→NF-κB p65 pathway, linking this inflammatory response with EMT and self-renewal of cancer cells (32-34). These pleotropic activities of the MUC1-C subunit are dependent on a CQC motif in the cytoplasmic domain that is necessary and sufficient for the formation of MUC1-C homodimers and their import into the nucleus (25, 35, 36).

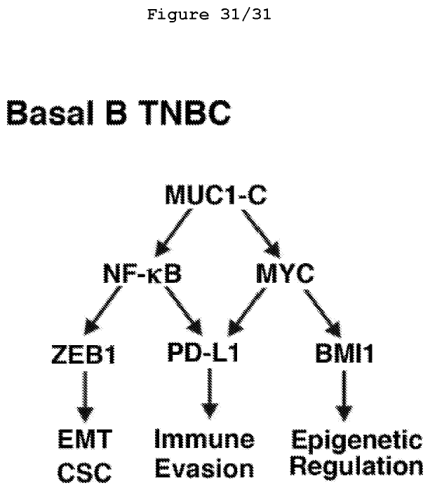

The present studies demonstrate that MUC1-C drives (i) constitutive PD-L1 expression in basal B BT-549, MDA-MB-231 and SUM-159 TNBC cells, which display mesenchymal and CSC characteristics (101-103) (FIG. 31), and (ii) inducible PD-L1 expression in basal A BT-20 TNBC cells. The results support a model in which MUC1-C activates the PD-L1 promoter in part by a MYC-dependent pathway. MUC1-C has been shown to activate MYC-mediated BMI1 expression and epigenetic alterations in basal B TNBC cells (98) (FIG. 31). Here, we show that targeting MUC1-C results in the downregulation of MYC, decreased occupancy of MYC on the PD-L1 promoter and suppression of pPD-L1-Luc reporter activation, all in support of a transcriptional mechanism. A MUC1-C→MYC→PD-L1 pathway was further supported by the findings that targeting MYC with inducible silencing or the JQ1 inhibitor suppresses PD-L1 expression.

The present results also demonstrate that MUC1-C induces PD-L1 by an NF-κB p65-mediated mechanism. Along these lines, MUC1-C activates the inflammatory NF-κB p65 pathway in basal B TNBC cells (32, 33, 44). MUC1-C binds directly to NF-κB p65 and promotes NF-κB p65 occupancy on its target gene, ZEB1, which in turn drives the ZEB1→miR-200c loop and the induction of EMT (33,44) (FIG. 31). In concert with those findings, we show here that targeting MUC1-C decreases NF-κB p65 occupancy on the PD-L1 promoter and suppresses activation of the pPD-L1-Luc reporter. Targeting NF-κB p65 also resulted in downregulation of PD-L1 expression, supporting activation of a MUC1-C→NF-κB p65→PD-L1 pathway. Of note, the MUC1-C→MYC and MUC1-C→NF-κB p65 pathways both have significant roles in driving PD-L1 expression in the basal B TNBC cells (FIG. 31), supporting potential cross-talk of these two transcription factors in activating the PD-L1 promoter.

As an extension of the studies in human TNBC cells, we established mouse Eo771 TNBC cells that stably express human MUC1-C and confirmed that MUC1-C induces PD-L1 expression in this model. The results further indicate that, as observed in human TNBC cells, MUC1-C-induced increases in PD-L1 in Eo771/MUC1-C cells are mediated by MYC and NF-κB. The Eo771/MUC1-C cells also provided an opportunity to assess the effects of targeting MUC1-C in immune competent MUC1.Tg mice bearing established Eo771/MUC1-C tumors. Notably, and in contrast to GO-203/NPs, anti-PD-L1 treatment had little if any effect on growth of the Eo771/MUC1-C tumors. Importantly, GO-203/NP treatment of Eo771/MUC1-C cells growing in vitro and as tumors in MUC1.Tg mice was associated with downregulation of PD-L1 expression. We also found that targeting MUC1-C and thereby suppression of PD-L1 in Eo771/MUC1-C tumors is associated with activation of the CD8+ Tcell population. In support of that contention, we found that GO-203/NP treatment results in upregulation of the CD69 activation marker and granzyme B in the CD8+ T-cell population. The CD8+ T-cells obtained from GO-203/NP-treated mice were also more effective in killing Eo771/MUC1-C cells. In patients with TNBCs treated with adjuvant or neoadjuvant chemotherapy, the presence of TILs is associated with improved clinical outcomes (82-84). Datasets obtained from TNBC patients were analyzed and, interestingly, found that MUC1 expression predicts for decreases in mRNA levels of intratumoral (i) CD8, and (ii) the CD69 and granzyme B markers of T-cell activation. In addition, the analysis of the databases showed that decreases in CD8, CD69 and GZMB expression each correlated with more aggressive disease. These findings and those in our in vitro and mouse model studies further support a role for MUC1-C in suppressing immune recognition and destruction.

The present invention and the studies described herein provide new insights into the integration of increased PD-L1 expression with the EMT process. In this way, MUC1-C drives EMT in basal B TNBC cells by activation of the inflammatory NF-κB p65 pathway and thereby induction of the EMT transcription factor ZEB1 (44) (FIG. 31). In turn, ZEB1 (i) promotes loss of polarity by suppression of polarity factors, such as CRB3, and (ii) activates the HIPPO/YAP pathway with induction of MYC in TNBC cells (24). ZEB1 also decreases expression of the miR-200c tumor suppressor, which is a negative regulator of PD-L1 (29). In this regard, recent work has demonstrated that MUC1-C increases PD-L1 expression in AML cells by suppression of miR-200c (107), supporting the premise that MUC1-C regulates PD-L1 by transcriptional and posttranscriptional mechanisms which are dependent on cell context. The MUC1-C→NF-κB p65 and MUC1-C→MYC pathways also have the capacity to induce epigenetic modifications needed for the associated changes in gene expression for the EMT program and CSC state (96, 98) (FIG. 31). Of potential interest is why PD-L1 and EMT would be integrated in basal B TNBC cells. One explanation is that invasive and metastatic cancer cells require a defense against immune recognition. Another possibility is that the overexpression of MUC1-C with induction of PDL1 and EMT represents an appropriation and exploitation by cancer cells of an epithelial stress response that evolved to repair damaged epithelia (69).

The present studies demonstrate that targeting MUC1-C in NSCLC cells is associated with downregulation of PD-L1 expression. Specially, MUC1-C induces PD-L1 transcription by forming MUC1-C/NF-κB p65 complexes on the PD-L1 promoter. Additionally, the present studies demonstrate that targeting MUC1-C results in derepression of TLR9, IFNG, MCP-1/CCL2 and GM-CSF/CSF2 gene expression by the ZEB1 transcriptional suppressor. These findings support the notion that MUC1-C is of importance for evasion of tumor cells to immune recognition and destruction.

Furthermore, the present studies also demonstrates that MUC1-C activates the CD274/PD-L1 gene in TNBC cells. The results presented herein (i) MUC1-C drives PD-L1 transcription by MYC- and NF-κB p65-mediated mechanisms, and (ii) targeting MUC1-C with genetic and pharmacologic approaches results in the suppression of PD-L1. Targeting MUC1-C in MUC1.Tg mice harboring mouse Eo771/MUC1-C tumors further showed suppression of PD-L1 by tumor cells and activation of the tumor immune microenvironment. These results and those from analysis of TNBC datasets provide additional support for involvement of MUC1-C in immune evasion of cancer

Accordingly, the invention features methods of de-repressing an anti-tumor immune response, increasing the efficacy of an immunotherapy regimen or augmenting presentation of tumor associated antigens by administering to the subject a MUC1 inhibitor, a MYC inhibitor, a TAK1 inhibitor, an NF-kβ p65 pathway inhibitor, an IKK inhibitor, or a ZEB1 pathway inhibitor.

Mucin-1 Inhibitors

A mucin-1 (MUC1) inhibitor is a compound that decreases expression or activity of MUC1. MUC1 is an oncogenic glycoprotein that is aberrantly expressed in many solid tumor and hematological malignancies including MM. MUC1 plays a vital role in supporting key aspects of the malignant phenotype including cell proliferation and self-renewal, resistance to cytotoxic injury and apoptosis, and capacity for migration and tissue invasion. MUC1 is comprised of an N-terminus that is shed into the circulation and a C-terminus that upon activation, undergoes homodimerization, translocation to the nucleus and interaction with downstream effectors including Wnt/b-catenin, NF-kB, and the JAK/STAT pathway. A MUC1 inhibitor decreases expression or activity of MUC1. A decrease in MUC1 activity is defined by a reduction of a biological function of the MUC1. For example, a decrease or reduction in MUC1 expression or biological activity refers to at least a 1%, 2%, 5%, 10%, 15%, 20%, 25%, 30%, 35%, 40%, 45%, 50%, 60%, 70%, 80%, 90% or 100% decrease in MUC1 expression or activity compared to a control.

A biological activity of a MUC1 inhibitor includes for example upregulation of miR-200c.

MUC1 expression is measured by detecting a MUC1 transcript or protein using standard methods known in the art, such as RT-PCR, microarray, and immunoblotting or immunohistochemistry with MUC1-specific antibodies. For example, a decrease in MUC1 expression refers to at least a 1%, 2%, 5%, 10%, 15%, 20%, 25%, 30%, 35%, 40%, 45%, 50%, 60%, 70%, 80%, 90% or 100% decrease in the level of MUC1 mRNA or MUC1 protein.

The MUC1 inhibitor is an antibody or fragment thereof specific to MUC1. Methods for designing and producing specific antibodies are well-known in the art. In particular embodiments the MUC1 inhibitor is a bi-specific antibody. For example, the bi-specific antibody is specific for MUC1 and PD-1 or PDL-1.

The MUC1 inhibitor can also be a small molecule. A “small molecule” as used herein, is meant to refer to a composition that has a molecular weight in the range of less than about 5 kD to 50 daltons, for example less than about 4 kD, less than about 3.5 kD, less than about 3 kD, less than about 2.5 kD, less than about 2 kD, less than about 1.5 kD, less than about 1 kD, less than 750 daltons, less than 500 daltons, less than about 450 daltons, less than about 400 daltons, less than about 350 daltons, less than 300 daltons, less than 250 daltons, less than about 200 daltons, less than about 150 daltons, less than about 100 daltons. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention. For example, the MUC1 inhibitor is G0-203.

Alternatively, the MUC1 inhibitor is for example an antisense MUC1 nucleic acid, a MUC1 specific short-interfering RNA, or a MUC1-specific ribozyme. By the term “siRNA” is meant a double stranded RNA molecule which prevents translation of a target mRNA. Standard techniques of introducing siRNA into a cell are used, including those in which DNA is a template from which an siRNA is transcribed. The siRNA includes a sense MUC1 nucleic acid sequence, an anti-sense MUC1 nucleic acid sequence or both. Optionally, the siRNA is constructed such that a single transcript has both the sense and complementary antisense sequences from the target gene, e.g., a hairpin (shRNA). Examples of siRNAs and shRNAs are disclosed in the examples herein.

Binding of the siRNA to a MUC1 transcript in the target cell results in a reduction in MUC1 production by the cell. The length of the oligonucleotide is at least 10 nucleotides and may be as long as the naturally-occurring MUC1 transcript. Preferably, the oligonucleotide is 19-25 nucleotides in length. Most preferably, the oligonucleotide is less than 75, 50, 25 nucleotides in length.

MYC Inhibitors

A MYC inhibitor is a compound that decreases expression or activity of MYC.

MYC protein is a transcription factor that activates expression of many genes through binding enhancer box sequences (E-boxes) and recruiting histone acetyltransferases (HATs). It can also act as a transcriptional repressor. By binding Miz-1 transcription factor and displacing the p300 co-activator, it inhibits expression of Miz-1 target genes. In addition, MYC has a direct role in the control of DNA replication

MYC is activated upon various mitogenic signals such as serum stimulation or by Wnt, Shh and EGF (via the MAPK/ERK pathway). By modifying the expression of its target genes, MYC activation results in numerous biological effects. The first to be discovered was its capability to drive cell proliferation (upregulates cyclins, downregulates p21), but it also plays a very important role in regulating cell growth (upregulates ribosomal RNA and proteins), apoptosis (downregulates Bcl-2), differentiation, and stem cell self-renewal. MYC is a very strong proto-oncogene and it is very often found to be upregulated in many types of cancers. MYC overexpression stimulates gene amplification, presumably through DNA over-replication.

A MYC inhibitor decreases expression or activity of MYC. A decrease in MYC activity is defined by a reduction of a biological function of the MYC. For example, a decrease or reduction in MYC expression or biological activity refers to at least a 1%, 2%, 5%, 10%, 15%, 20%, 25%, 30%, 35%, 40%, 45%, 50%, 60%, 70%, 80%, 90% or 100% decrease in MYC expression or activity compared to a control.

A biological activity of a MYC inhibitor includes for example upregulation of miR-200c.

MYC expression is measured by detecting a MYC transcript or protein using standard methods known in the art, such as RT-PCR, microarray, and immunoblotting or immunohistochemistry with MYC-specific antibodies. For example, a decrease in MYC expression refers to at least a 1%, 2%, 5%, 10%, 15%, 20%, 25%, 30%, 35%, 40%, 45%, 50%, 60%, 70%, 80%, 90% or 100% decrease in the level of MYC mRNA or MUC1 protein.

The MYC inhibitor is an antibody or fragment thereof specific to MYC. Methods for designing and producing specific antibodies are well-known in the art. In particular embodiments the MYC inhibitor is a bi-specific antibody. For example, the bi-specific antibody is specific for MYC and PD-1 or PDL-1.

The MYC inhibitor can also be a small molecule. A “small molecule” as used herein, is meant to refer to a composition that has a molecular weight in the range of less than about 5 kD to 50 daltons, for example less than about 4 kD, less than about 3.5 kD, less than about 3 kD, less than about 2.5 kD, less than about 2 kD, less than about 1.5 kD, less than about 1 kD, less than 750 daltons, less than 500 daltons, less than about 450 daltons, less than about 400 daltons, less than about 350 daltons, less than 300 daltons, less than 250 daltons, less than about 200 daltons, less than about 150 daltons, less than about 100 daltons. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention. For example, the MYC inhibitor is 10074-G5 or 10058-F4.

Alternatively, the MUC1 inhibitor is for example an antisense MYC nucleic acid, a MYC specific short-interfering RNA, or a MYC-specific ribozyme. By the term “siRNA” is meant a double stranded RNA molecule which prevents translation of a target mRNA. Standard techniques of introducing siRNA into a cell are used, including those in which DNA is a template from which an siRNA is transcribed. The siRNA includes a sense MYC nucleic acid sequence, an anti-sense MYC nucleic acid sequence or both. Optionally, the siRNA is constructed such that a single transcript has both the sense and complementary antisense sequences from the target gene, e.g., a hairpin (shRNA). Examples of siRNAs and shRNAs are disclosed in the examples herein.

Binding of the siRNA to a MYC transcript in the target cell results in a reduction in MYC production by the cell. The length of the oligonucleotide is at least 10 nucleotides and may be as long as the naturally-occurring MYC transcript. Preferably, the oligonucleotide is 19-25 nucleotides in length. Most preferably, the oligonucleotide is less than 75, 50, 25 nucleotides in length.

TGF-Beta Activated Kinase 1 Inhibitors

A TGF-beta activated kinase 1 (TAK1) inhibitor is a compound that decreases expression or activity of TAK1. TAK1 is a signaling intermediate in tumor necrosis factor (TNF), interleukin 1, and Toll-like receptor signaling pathways. TAK1-binding protein 2 (TAB2) and its closely related protein, TAB3, are binding partners of TAK1 and have previously been identified as adaptors of TAK1 that recruit TAK1 to a TNF receptor signaling complex. TAB2 and TAB3 redundantly mediate activation of TAK1A.

TAK1 inhibitor decreases expression or activity of TAK1. A decrease in TAK1 activity is defined by a reduction of a biological function of the TAK1. For example, a decrease or reduction in TAK1 expression or biological activity refers to at least a 1%, 2%, 5%, 10%, 15%, 20%, 25%, 30%, 35%, 40%, 45%, 50%, 60%, 70%, 80%, 90% or 100% decrease in TAK1 expression or activity compared to a control.

A biological activity of a TAK1 inhibitor includes for example B cell receptor crosslinking.

TAK1 expression is measured by detecting a TAK1 transcript or protein using standard methods known in the art, such as RT-PCR, microarray, and immunoblotting or immunohistochemistry with TAK1-specific antibodies. For example, a decrease in TAK1 expression refers to at least a 1%, 2%, 5%, 10%, 15%, 20%, 25%, 30%, 35%, 40%, 45%, 50%, 60%, 70%, 80%, 90% or 100% decrease in the level of TAK1 mRNA or TAK1 protein.

The TAK1 inhibitor is an antibody or fragment thereof specific to TAK1. Methods for designing and producing specific antibodies are well-known in the art. In particular embodiments the TAK1 inhibitor is a bi-specific antibody. For example, the bi-specific antibody is specific for TAK1 and PD-1 or PDL-1.

The TAK1 inhibitor can also be a small molecule. A “small molecule” as used herein, is meant to refer to a composition that has a molecular weight in the range of less than about 5 kD to 50 daltons, for example less than about 4 kD, less than about 3.5 kD, less than about 3 kD, less than about 2.5 kD, less than about 2 kD, less than about 1.5 kD, less than about 1 kD, less than 750 daltons, less than 500 daltons, less than about 450 daltons, less than about 400 daltons, less than about 350 daltons, less than 300 daltons, less than 250 daltons, less than about 200 daltons, less than about 150 daltons, less than about 100 daltons. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention. For example, the TAK1 inhibitor is (5Z)-7-Oxozeaenol.

Alternatively, the TAK1 inhibitor is for example an antisense TAK1 nucleic acid, a TAK1 specific short-interfering RNA, or a TAK1-specific ribozyme. By the term “siRNA” is meant a double stranded RNA molecule which prevents translation of a target mRNA. Standard techniques of introducing siRNA into a cell are used, including those in which DNA is a template from which an siRNA is transcribed. The siRNA includes a sense TAK1 nucleic acid sequence, an anti-sense TAK1 nucleic acid sequence or both. Optionally, the siRNA is constructed such that a single transcript has both the sense and complementary antisense sequences from the target gene, e.g., a hairpin (shRNA). Examples of siRNAs and shRNAs are disclosed in the examples herein.

Binding of the siRNA to a TAK1 transcript in the target cell results in a reduction in TAK1 production by the cell. The length of the oligonucleotide is at least 10 nucleotides and may be as long as the naturally-occurring TAK1 transcript. Preferably, the oligonucleotide is 19-25 nucleotides in length. Most preferably, the oligonucleotide is less than 75, 50, 25 nucleotides in length.

NFκ-β p65 Pathway Inhibitors

Nuclear factor-κB (NFκ-β) signaling pathway plays a major role in the development, maintenance, and progression of most chronic diseases. NFκ-β controls the expression of genes involved in a number of physiological responses, including immune inflammatory responses, acute-phase inflammatory responses, oxidative stress responses, cell adhesion, differentiation, and apoptosis.

More than 700 inhibitors of the NF-κB activation pathway, including antioxidants, peptides, small RNA/DNA, microbial and viral proteins, small molecules, and engineered dominant-negative or constitutively active polypeptides have been described. (See, Gupta, S. Biochim Biophys Acta. 2010 October-December; 1799(10-12): 775-787, the content of which are incorporated by reference in its entirety.

The NFκ-β p65 pathway inhibitor is an antibody or fragment thereof specific to NFκ-β or p65. Methods for designing and producing specific antibodies are well-known in the art. In particular embodiments the NFκ-β p65 pathway inhibitor is a bi-specific antibody. For example, the bi-specific antibody is specific for NFκ-β or p65 and PD-1 or PDL-1.

The NFκ-β p65 pathway inhibitor can also be a small molecule. A “small molecule” as used herein, is meant to refer to a composition that has a molecular weight in the range of less than about 5 kD to 50 daltons, for example less than about 4 kD, less than about 3.5 kD, less than about 3 kD, less than about 2.5 kD, less than about 2 kD, less than about 1.5 kD, less than about 1 kD, less than 750 daltons, less than 500 daltons, less than about 450 daltons, less than about 400 daltons, less than about 350 daltons, less than 300 daltons, less than 250 daltons, less than about 200 daltons, less than about 150 daltons, less than about 100 daltons. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention. For example, the NFκ-β p65 pathway inhibitor is BAY-11-7085, SB203580 or PD0980589.

Alternatively, the NFκ-β p65 pathway inhibitor is for example an antisense nucleic acid, a specific short-interfering RNA, or a ribozyme. By the term “siRNA” is meant a double stranded RNA molecule which prevents translation of a target mRNA. Standard techniques of introducing siRNA into a cell are used, including those in which DNA is a template from which an siRNA is transcribed. The siRNA includes a sense nucleic acid sequence, an anti-sense nucleic acid sequence or both. Optionally, the siRNA is constructed such that a single transcript has both the sense and complementary antisense sequences from the target gene, e.g., a hairpin (shRNA). Examples of siRNAs and shRNAs are disclosed in the examples herein. The length of the oligonucleotide is at least 10 nucleotides and may be as long as the naturally-occurring TAK1 transcript. Preferably, the oligonucleotide is 19-25 nucleotides in length. Most preferably, the oligonucleotide is less than 75, 50, 25 nucleotides in length.

IκB Kinase Inhibitor

The IκB kinase (IKK) is an enzyme complex that is involved in propagating the cellular response to inflammation. The IκB kinase enzyme complex is part of the upstream NF-κB signal transduction cascade. The IκBα (inhibitor of kappa B) protein inactivates the NF-κB transcription factor by masking the nuclear localization signals (NLS) of NF-κB proteins and keeping them sequestered in an inactive state in the cytoplasm. Specifically, IKK phosphorylates the inhibitory IκBα protein.

An IKK inhibitor decreases expression or activity of IKK. A decrease in IKK activity is defined by a reduction of a biological function of the IKK. For example, a decrease or reduction in IKK expression or biological activity refers to at least a 1%, 2%, 5%, 10%, 15%, 20%, 25%, 30%, 35%, 40%, 45%, 50%, 60%, 70%, 80%, 90% or 100% decrease in IKK expression or activity compared to a control.

A biological activity of a IKK inhibitor includes for example activation of NFκ-β.

IKK expression is measured by detecting a IKK transcript or protein using standard methods known in the art, such as RT-PCR, microarray, and immunoblotting or immunohistochemistry with IKK-specific antibodies. For example, a decrease in IKK expression refers to at least a 1%, 2%, 5%, 10%, 15%, 20%, 25%, 30%, 35%, 40%, 45%, 50%, 60%, 70%, 80%, 90% or 100% decrease in the level of IKK mRNA or IKK protein.

The IKK inhibitor is an antibody or fragment thereof specific to IKK. Methods for designing and producing specific antibodies are well-known in the art. In particular embodiments the IKK inhibitor is a bi-specific antibody. For example, the bi-specific antibody is specific for IKK and PD-1 or PDL-1.

The IKK inhibitor can also be a small molecule. A “small molecule” as used herein, is meant to refer to a composition that has a molecular weight in the range of less than about 5 kD to 50 daltons, for example less than about 4 kD, less than about 3.5 kD, less than about 3 kD, less than about 2.5 kD, less than about 2 kD, less than about 1.5 kD, less than about 1 kD, less than 750 daltons, less than 500 daltons, less than about 450 daltons, less than about 400 daltons, less than about 350 daltons, less than 300 daltons, less than 250 daltons, less than about 200 daltons, less than about 150 daltons, less than about 100 daltons. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention. For example, the IKK inhibitor is Bay 11-7082.

Alternatively, the IKK inhibitor is for example an antisense IKK nucleic acid, a IKK specific short-interfering RNA, or a IKK-specific ribozyme. By the term “siRNA” is meant a double stranded RNA molecule which prevents translation of a target mRNA. Standard techniques of introducing siRNA into a cell are used, including those in which DNA is a template from which an siRNA is transcribed. The siRNA includes a sense T IKK nucleic acid sequence, an anti-sense IKK nucleic acid sequence or both. Optionally, the siRNA is constructed such that a single transcript has both the sense and complementary antisense sequences from the target gene, e.g., a hairpin (shRNA). Examples of siRNAs and shRNAs are disclosed in the examples herein.

Binding of the siRNA to a IKK transcript in the target cell results in a reduction in IKK production by the cell. The length of the oligonucleotide is at least 10 nucleotides and may be as long as the naturally-occurring IKK transcript. Preferably, the oligonucleotide is 19-25 nucleotides in length. Most preferably, the oligonucleotide is less than 75, 50, 25 nucleotides in length.

Zinc Finger E-Box-Binding Homeobox 1 Inhibitor

Zinc finger E-box-binding homeobox 1 (ZEB1) (previously known as TCF8) encodes a zinc finger and homeodomain transcription factor that represses T-lymphocyte-specific IL2 gene expression by binding to a negative regulatory domain 100 nucleotides 5-prime of the IL2 transcription start site.

A ZEB1 inhibitor decreases expression or activity of ZEB1. A decrease in ZEB1 activity is defined by a reduction of a biological function of the ZEB1. For example, a decrease or reduction in ZEB1 expression or biological activity refers to at least a 1%, 2%, 5%, 10%, 15%, 20%, 25%, 30%, 35%, 40%, 45%, 50%, 60%, 70%, 80%, 90% or 100% decrease in ZEB1 expression or activity compared to a control.

A biological activity of a ZEB1 inhibitor includes for example activation of NF-κB.

ZEB1 expression is measured by detecting a ZEB1 transcript or protein using standard methods known in the art, such as RT-PCR, microarray, and immunoblotting or immunohistochemistry with ZEB1-specific antibodies. For example, a decrease in ZEB1 expression refers to at least a 1%, 2%, 5%, 10%, 15%, 20%, 25%, 30%, 35%, 40%, 45%, 50%, 60%, 70%, 80%, 90% or 100% decrease in the level of ZEB1 mRNA or ZEB1 protein.

The ZEB1 inhibitor is an antibody or fragment thereof specific to ZEB1. Methods for designing and producing specific antibodies are well-known in the art. In particular embodiments the ZEB1 inhibitor is a bi-specific antibody. For example, the bi-specific antibody is specific for ZEB1 and PD-1 or PDL-1.

The ZEB1 inhibitor can also be a small molecule. A “small molecule” as used herein, is meant to refer to a composition that has a molecular weight in the range of less than about 5 kD to 50 daltons, for example less than about 4 kD, less than about 3.5 kD, less than about 3 kD, less than about 2.5 kD, less than about 2 kD, less than about 1.5 kD, less than about 1 kD, less than 750 daltons, less than 500 daltons, less than about 450 daltons, less than about 400 daltons, less than about 350 daltons, less than 300 daltons, less than 250 daltons, less than about 200 daltons, less than about 150 daltons, less than about 100 daltons. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

Alternatively, the ZEB1 inhibitor is for example an antisense ZEB1 nucleic acid, a ZEB1 specific short-interfering RNA, or a ZEB1-specific ribozyme. By the term “siRNA” is meant a double stranded RNA molecule which prevents translation of a target mRNA. Standard techniques of introducing siRNA into a cell are used, including those in which DNA is a template from which an siRNA is transcribed. The siRNA includes a sense T ZEB1 nucleic acid sequence, an anti-sense ZEB1 nucleic acid sequence or both. Optionally, the siRNA is constructed such that a single transcript has both the sense and complementary antisense sequences from the target gene, e.g., a hairpin (shRNA). Examples of siRNAs and shRNAs are disclosed in the examples herein.

Binding of the siRNA to a ZEB1 transcript in the target cell results in a reduction in ZEB1 production by the cell. The length of the oligonucleotide is at least 10 nucleotides and may be as long as the naturally-occurring ZEB1 transcript. Preferably, the oligonucleotide is 19-25 nucleotides in length. Most preferably, the oligonucleotide is less than 75, 50, 25 nucleotides in length.

Therapeutic Methods

In various aspects, the invention provides methods of treating cancer in a subject. The method includes administering to the subject a compound that inhibits the expression or activity of MUC1, MYC, TAK1, the NF-kB-p65 pathway, IKK inhibitor, or the ZEB1 pathway. The inhibitor is administered in an amount sufficient to decrease tumor PD-L1 transcription and or TLR7 transcription. Alternatively, inhibitor is administered in an amount sufficient to increase CD8, CD69, GZMB, TLR9, IFNγ, MCP-1 or GM-CSF expression. The inhibitor is administered in an amount sufficient to increase the expression of TAP-1, TAP-2, MHC or Tapasin.

Cells are directly contacted with the inhibitor. Alternatively, the inhibitor is administered systemically.

Cancer is treated by de-repressing an anti-tumor immune response. The immune response is an innate immune response or an adaptive immune response.

Alternatively, cancer is treated by increasing the efficacy of an immunotherapy regimen. Immunotherapy includes for example therapeutic antibody, a CAR T-cell therapy, a dendritic cell/tumor fusion, or a tumor vaccine.

In other aspects of the invention, cancer is treated by augmenting presentation of tumor associated antigens.

The subject will receive, has received or is receiving therapeutic antibody. Therapeutic antibodies include for example, Alemtuzumab, Atezolizumab, Ipilimumab Nivolumab, Ofatumumab, Pembrolizumab, or Rituximab.

The subject will receive, has received or is receiving checkpoint inhibitor therapy. By checkpoint inhibitor it is meant that at the compound inhibits a protein in the checkpoint signally pathway. Proteins in the checkpoint signally pathway include for example, PD-1, PD-L1, PD-L2, CTLA-4, LAG-3, B7-H3, B7-H4, Tim3, BTLA, KIR, A2aR, and/or CD200. Checkpoint inhibitor are known in the art. For example, the checkpoint inhibitor can be a small molecule. A “small molecule” as used herein, is meant to refer to a composition that has a molecular weight in the range of less than about 5 kD to 50 daltons, for example less than about 4 kD, less than about 3.5 kD, less than about 3 kD, less than about 2.5 kD, less than about 2 kD, less than about 1.5 kD, less than about 1 kD, less than 750 daltons, less than 500 daltons, less than about 450 daltons, less than about 400 daltons, less than about 350 daltons, less than 300 daltons, less than 250 daltons, less than about 200 daltons, less than about 150 daltons, less than about 100 daltons. Small molecules can be, e.g., nucleic acids, peptides, polypeptides, peptidomimetics, carbohydrates, lipids or other organic or inorganic molecules.

Alternatively the checkpoint inhibitor is an antibody is an antibody or fragment thereof. For example, the antibody or fragment thereof is specific to a protein in the checkpoint signaling pathway, such as PD-1, PD-L1, PD-L2, CTLA-4, LAG-3, B7-H3, B7-H4, Tim3, BTLA, KIR, A2aR, and/or CD200.

The subject will receive, has received or is receiving a tumor vaccine consisting of a fusion between autologous dendritic cells (DCs) and tumor cells (DC cell fusions).

The subject will receive, has received or is receiving CAR T-cell therapy.

Optionally, the patient may receive concurrent treatment with an immunomodulatory agent. These agents include lenalidomide, pomalinomide or apremilast. Lenalidomide has been shown to boost response to vaccination targeting infectious diseases and in pre-clinical studies enhances T cell response to a DC cell fusion vaccine.

The methods described herein are useful to alleviate the symptoms of a variety of cancers. The cancer is a solid tumor or a hematologic tumor. The solid tumor is for example a lung tumor, a breast tumor, or a renal tumor. The hematologic tumor id for example acute myeloid leukemia (AML) or multiple myeloma (MM).

Treatment is efficacious if the treatment leads to clinical benefit such as, a decrease in size, prevalence, or metastatic potential of the tumor in the subject. When treatment is applied prophylactically, “efficacious” means that the treatment retards or prevents tumors from forming or prevents or alleviates a symptom of clinical symptom of the tumor. Efficaciousness is determined in association with any known method for diagnosing or treating the particular tumor type.

Therapeutic Administration

The invention includes administering to a subject composition comprising a MUC1 inhibitor, a MYC a TAK1 inhibitor, an NF-kB p65 pathway inhibitor, an IKK inhibitor, or a ZEB1 pathway inhibitor.

An effective amount of a therapeutic compound is preferably from about 0.1 mg/kg to about 150 mg/kg. Effective doses vary, as recognized by those skilled in the art, depending on route of administration, excipient usage, and coadministration with other therapeutic treatments including use of other anti-proliferative agents or therapeutic agents for treating, preventing or alleviating a symptom of a cancer. A therapeutic regimen is carried out by identifying a mammal, e.g., a human patient suffering from a cancer using standard methods.

Doses may be administered once, or more than once. In some embodiments, it is preferred that the therapeutic compound is administered once a week, twice a week, three times a week, four times a week, five times a week, six times a week, or seven times a week for a predetermined duration of time. The predetermined duration of time may be 1 week, 2 weeks, 3 weeks, 4 weeks, 5 weeks, 6 weeks, 7 weeks, 2 months, 3 months, 4 months, 5 months, 6 months, 7 months, 8 months, 9 months, 10 months, 11 months, or up to 1 year.

The pharmaceutical compound is administered to such an individual using methods known in the art. Preferably, the compound is administered orally, rectally, nasally, topically or parenterally, e.g., subcutaneously, intraperitoneally, intramuscularly, and intravenously. The inhibitors are optionally formulated as a component of a cocktail of therapeutic drugs to treat cancers. Examples of formulations suitable for parenteral administration include aqueous solutions of the active agent in an isotonic saline solution, a 5% glucose solution, or another standard pharmaceutically acceptable excipient. Standard solubilizing agents such as PVP or cyclodextrins are also utilized as pharmaceutical excipients for delivery of the therapeutic compounds.

The therapeutic compounds described herein are formulated into compositions for other routes of administration utilizing conventional methods. For example, the therapeutic compounds are formulated in a capsule or a tablet for oral administration. Capsules may contain any standard pharmaceutically acceptable materials such as gelatin or cellulose. Tablets may be formulated in accordance with conventional procedures by compressing mixtures of a therapeutic compound with a solid carrier and a lubricant. Examples of solid carriers include starch and sugar bentonite. The compound is administered in the form of a hard shell tablet or a capsule containing a binder, e.g., lactose or mannitol, conventional filler, and a tableting agent. Other formulations include an ointment, suppository, paste, spray, patch, cream, gel, resorbable sponge, or foam. Such formulations are produced using methods well known in the art.

The therapeutic compounds described herein may be formulated into nanoparticles such as polymeric nanoparticles. In a particular embodiment GO-203 is formulated in polymeric nanoparticles

Therapeutic compounds are effective upon direct contact of the compound with the affected tissue. Accordingly, the compound is administered topically. Alternatively, the therapeutic compounds are administered systemically. For example, the compounds are administered by inhalation. The compounds are delivered in the form of an aerosol spray from pressured container or dispenser which contains a suitable propellant, e.g., a gas such as carbon dioxide, or a nebulizer.

Additionally, compounds are administered by implanting (either directly into an organ or subcutaneously) a solid or resorbable matrix which slowly releases the compound into adjacent and surrounding tissues of the subject.

In some embodiments, it is preferred that the therapeutic compounds described herein are administered in combination with another therapeutic agent, such as a chemotherapeutic agent, radiation therapy, or an anti-mitotic agent. In some aspects, the anti-mitotic agent is administered prior to administration of the present therapeutic compound, in order to induce additional chromosomal instability to increase the efficacy of the present invention to targeting cancer cells. Examples of anti-mitotic agents include taxanes (i.e., paclitaxel, docetaxel), and vinca alkaloids (i.e., vinblastine, vincristine, vindesine, vinorelbine).

### Definitions

The practice of the present invention employs, unless otherwise indicated, conventional techniques of molecular biology, microbiology, cell biology and recombinant DNA, which are within the skill of the art. See, e.g., Sambrook, Fritsch and Maniatis, MOLECULAR CLONING: A LABORATORY MANUAL, 2^{nd} edition (1989); CURRENT PROTOCOLS IN MOLECULAR BIOLOGY (F. M. Ausubel et al. eds., (1987)); the series METHODS IN ENZYMOLOGY (Academic Press, Inc.): PCR 2: A PRACTICAL APPROACH (Mi. MacPherson, B. D. Hames and G. R. Taylor eds. (1995)) and ANIMAL CELL CULTURE (Rd. Freshney, ed. (1987)).

As used herein, certain terms have the following defined meanings. As used in the specification and claims, the singular form “a”, “an” and “the” include plural references unless the context clearly dictates otherwise. For example, the term “a cell” includes a plurality of cells, including mixtures thereof.

“Treatment” is an intervention performed with the intention of preventing the development or altering the pathology or symptoms of a disorder. Accordingly, “treatment” refers to both therapeutic treatment and prophylactic or preventative measures. Those in need of treatment include those already with the disorder as well as those in which the disorder is to be prevented. In tumor (e.g., cancer) treatment, a therapeutic agent may directly decrease the pathology of tumor cells, or render the tumor cells more susceptible to treatment by other therapeutic agents, e.g., radiation and/or chemotherapy. As used herein, “ameliorated” or “treatment” refers to a symptom which is approaches a normalized value (for example a value obtained in a healthy patient or individual), e.g., is less than 50% different from a normalized value, preferably is less than about 25% different from a normalized value, more preferably, is less than 10% different from a normalized value, and still more preferably, is not significantly different from a normalized value as determined using routine statistical tests.

Thus, treating may include suppressing, inhibiting, preventing, treating, or a combination thereof. Treating refers inter alia to increasing time to sustained progression, expediting remission, inducing remission, augmenting remission, speeding recovery, increasing efficacy of or decreasing resistance to alternative therapeutics, or a combination thereof. “Suppressing” or “inhibiting”, refers inter alia to delaying the onset of symptoms, preventing relapse to a disease, decreasing the number or frequency of relapse episodes, increasing latency between symptomatic episodes, reducing the severity of symptoms, reducing the severity of an acute episode, reducing the number of symptoms, reducing the incidence of disease-related symptoms, reducing the latency of symptoms, ameliorating symptoms, reducing secondary symptoms, reducing secondary infections, prolonging patient survival, or a combination thereof. The symptoms are primary, while in another embodiment, symptoms are secondary. “Primary” refers to a symptom that is a direct result of the proliferative disorder, while, secondary refers to a symptom that is derived from or consequent to a primary cause. Symptoms may be any manifestation of a disease or pathological condition.

The “treatment of cancer or tumor cells”, refers to an amount of peptide or nucleic acid, described throughout the specification, capable of invoking one or more of the following effects: (1) inhibition of tumor growth, including, (i) slowing down and (ii) complete growth arrest; (2) reduction in the number of tumor cells; (3) maintaining tumor size; (4) reduction in tumor size; (5) inhibition, including (i) reduction, (ii) slowing down or (iii) complete prevention, of tumor cell infiltration into peripheral organs; (6) inhibition, including (i) reduction, (ii) slowing down or (iii) complete prevention, of metastasis; (7) enhancement of anti-tumor immune response, which may result in (i) maintaining tumor size, (ii) reducing tumor size, (iii) slowing the growth of a tumor, (iv) reducing, slowing or preventing invasion and/or (8) relief, to some extent, of the severity or number of one or more symptoms associated with the disorder.

As used herein, “an ameliorated symptom” or “treated symptom” refers to a symptom which approaches a normalized value, e.g., is less than 50% different from a normalized value, preferably is less than about 25% different from a normalized value, more preferably, is less than 10% different from a normalized value, and still more preferably, is not significantly different from a normalized value as determined using routine statistical tests.

The terms “patient” or “individual” are used interchangeably herein, and refers to a mammalian subject to be treated, with human patients being preferred. In some cases, the methods of the invention find use in experimental animals, in veterinary application, and in the development of animal models for disease, including, but not limited to, rodents including mice, rats, and hamsters; and primates.

As used herein, the term “anti-tumor immunity” refers to an immune response induced upon recognition of cancer antigens by immune cells.

As used herein, the term “T cell activation” refers to cellular activation of resting T cells manifesting a variety of responses (For example, T cell proliferation, cytokine secretion and/or effector function). T cell activation may be induced by stimulation of the T cell receptor (TCR) with antigen/MHC complex.

As used herein, the term “antigen presenting capacity” refers to the ability of antigen presenting cells (APCs) to present antigen to T lymphocytes to elicit an immune response. In certain embodiments, the immune response is a type I immunity response. In certain embodiments, the antigen presenting capacity is determined by measuring infiltration and activation of T cells at tumor locations and/or secretion of IFN-.gamma. and Granzyme B ex vivo by APCs (i.e., dendritic cells).

As used herein, the term “anti-tumor T cells” refers to T lymphocytes that have been activated by APCs, wherein the antigen is a tumor-associated antigen. These T lymphocytes will subsequently induce the killing of malignant cells.

As used herein, the term “anti-tumor response” refers to at least one of the following: tumor necrosis, tumor regression, tumor inflammation, tumor infiltration by activated T lymphocytes, or activation of tumor infiltrating lymphocytes. In certain embodiments, activation of lymphocytes is due to presentation of a tumor-associated antigen by APCs.

As used herein, the term “extended survival” refers to increasing overall or progression free survival in a treated subject relative to an untreated control.

As used herein, the terms “improved therapeutic outcome” and “enhanced therapeutic efficacy,” relative to cancer refers to a slowing or diminution of the growth of cancer cells or a solid tumor, or a reduction in the total number of cancer cells or total tumor burden. An “improved therapeutic outcome” or “enhanced therapeutic efficacy” therefore means there is an improvement in the condition of the patient according to any clinically acceptable criteria, including, for example, decreased tumor size, an increase in time to tumor progression, increased progression-free survival, increased overall survival time, an increase in life expectancy, or an improvement in quality of life. In particular, “improved” or “enhanced” refers to an improvement or enhancement of 1%, 5%, 10%, 25% 50%, 75%, 100%, or greater than 100% of any clinically acceptable indicator of therapeutic outcome or efficacy.

The terms “cancer”, “tumor”, “cancerous”, and “malignant” refer to or describe the physiological condition in mammals that is typically characterized by unregulated cell growth. Examples of cancer include but are not limited to, carcinoma including adenocarcinoma, lymphoma, blastoma, melanoma, sarcoma, and leukemia. More particular examples of such cancers include squamous cell cancer, small-cell lung cancer, non-small cell lung cancer, gastrointestinal cancer, Hodgkin's and non-Hodgkin's lymphoma, pancreatic cancer, glioblastoma, glioma, cervical cancer, ovarian cancer, liver cancer such as hepatic carcinoma and hepatoma, bladder cancer, breast cancer, including triple negative breast cancer, colon cancer, colorectal cancer, endometrial carcinoma, myeloma (such as multiple myeloma), salivary gland carcinoma, kidney cancer such as renal cell carcinoma and Wilms' tumors, basal cell carcinoma, melanoma, prostate cancer, vulval cancer, thyroid cancer, testicular cancer, esophageal cancer, and various types of head and neck cancer.

“Tumor burden” also referred to as “tumor load”, refers to the total amount of tumor material distributed throughout the body. Tumor burden refers to the total number of cancer cells or the total size of tumor(s), throughout the body, including lymph nodes and bone barrow. Tumor burden can be determined by a variety of methods known in the art, such as, e.g. by measuring the dimensions of tumor(s) upon removal from the subject, e.g., using calipers, or while in the body using imaging techniques, e.g., ultrasound, bone scan, computed tomography (CT) or magnetic resonance imaging (MRI) scans.

The term “tumor size” refers to the total size of the tumor which can be measured as the length and width of a tumor. Tumor size may be determined by a variety of methods known in the art, such as, e.g. by measuring the dimensions of tumor(s) upon removal from the subject, e.g., using calipers, or while in the body using imaging techniques, e.g., bone scan, ultrasound, CT or MRI scans.

As used herein, the term “primary cancer” refers to the original tumor or the first tumor. Cancer may begin in any organ or tissue of the body. It is usually named for the part of the body or the type of cell in which it originates (Metastatic Cancer: Questions and Answers, Cancer Facts 6.20, National Cancer Institute, reviewed Sep. 1, 2004 (2004)).

By the term “modulate,” it is meant that any of the mentioned activities, are, e.g., increased, enhanced, increased, augmented, agonized (acts as an agonist), promoted, decreased, reduced, suppressed blocked, or antagonized (acts as an antagonist). Modulation can increase activity more than 1-fold, 2-fold, 3-fold, 5-fold, 10-fold, 100-fold, etc., over baseline values. Modulation can also decrease its activity below baseline values.

As used herein, the term “administering to a cell” (e.g., an expression vector, nucleic acid, a delivery vehicle, agent, and the like) refers to transducing, transfecting, microinjecting, electroporating, or shooting, the cell with the molecule. In some aspects, molecules are introduced into a target cell by contacting the target cell with a delivery cell (e.g., by cell fusion or by lysing the delivery cell when it is in proximity to the target cell).

Dendritic cells (DCs) are potent APCs. DCs are minor constituents of various immune organs such as spleen, thymus, lymph node, epidermis, and peripheral blood. For instance, DCs represent merely about 1% of crude spleen (see Steinman et al. (1979) J. Exp. Med 149: 1) or epidermal cell suspensions (see Schuler et al. (1985) J. Exp. Med 161:526; Romani et al. J. Invest. Dermatol (1989) 93: 600) and 0.1-1% of mononuclear cells in peripheral blood (see Freudenthal et al. Proc. Natl Acad Sci USA (1990) 87: 7698). Methods for isolating DCs from peripheral blood or bone marrow progenitors are known in the art. (See Inaba et al. (1992) J. Exp. Med 175:1157; Inaba et al. (1992) J. Exp, Med 176: 1693-1702; Romani et al. (1994) J. Exp. Med. 180: 83-93; Sallusto et al. (1994) J. Exp. Med 179: 1109-1118)). Preferred methods for isolation and culturing of DCs are described in Bender et al. (1996) J. Immun. Meth. 196:121-135 and Romani et al. (1996) J. Immun. Meth 196:137-151. As used herein, the term “dendritic cell” refers to a type of specialized antigen presenting cell (APC) involved in innate and adaptive immunity. Also referred to as “DC.” Dendritic cells may be present in the tumor microenvironment and these are referred to as “tumor-associated dendritic cells” or “tDCs.”

Thus, the term “cytokine” refers to any of the numerous factors that exert a variety of effects on cells, for example, inducing growth or proliferation. Non-limiting examples of cytokines include, IL-2, stem cell factor (SCF), IL-3, IL-6, IL-7, IL-12, IL-15, G-CSF, GM-CSF, IL-1α, IL-1β, MIP-1α, LIF, c-kit ligand, TPO, and flt3 ligand. Cytokines are commercially available from several vendors such as, for example, Genzyme Corp. (Framingham, Mass.), Genentech (South San Francisco, Calif.), Amgen (Thousand Oaks, Calif.) and Immunex (Seattle, Wash.). It is intended, although not always explicitly stated, that molecules having similar biological activity as wild-type or purified cytokines (e.g., recombinantly produced cytokines) are intended to be used within the spirit and scope of the invention and therefore are substitutes for wild-type or purified cytokines.

“Costimulatory molecules” are involved in the interaction between receptor-ligand pairs expressed on the surface of antigen presenting cells and T cells. One exemplary receptor-ligand pair is the B7 co-stimulatory molecules on the surface of DCs and its counter-receptor CD28 or CTLA-4 on T cells. (See Freeman et al. (1993) Science 262:909-911; Young et al. (1992) J. Clin. Invest 90: 229; Nabavi et al. Nature 360:266)). Other important costimulatory molecules include, for example, CD40, CD54, CD80, and CD86. These are commercially available from vendors identified above.

As used herein, an “immune modulating agent” is an agent capable of altering the immune response of a subject. In certain embodiments, “immune modulating agents” include adjuvants (substances that enhance the body's immune response to an antigen), vaccines (e.g., cancer vaccines), and those agents capable of altering the function of immune checkpoints, including the CTLA-4, LAG-3, B7-H3, B7-H4, Tim3, BTLA, KIR, A2aR, CD200 and/or PD-1 pathways. Exemplary immune checkpoint modulating agents include anti-CTLA-4 antibody (e.g., ipilimumab), anti-LAG-3 antibody, anti-B7-H3 antibody, anti-B7-H4 antibody, anti-Tim3 antibody, anti-BTLA antibody, anti-KIR antibody, anti-A2aR antibody, anti CD200 antibody, anti-PD-1 antibody, anti-PD-L1 antibody, anti-CD28 antibody, anti-CD80 or -CD86 antibody, anti-B7RP1 antibody, anti-B7-H3 antibody, anti-HVEM antibody, anti-CD137 or -CD137L antibody, anti-OX40 or -OX40L antibody, anti-CD40 or -CD40L antibody, anti-GAL9 antibody, anti-IL-10 antibody and A2aR drug. For certain such immune pathway gene products, the use of either antagonists or agonists of such gene products is contemplated, as are small molecule modulators of such gene products. In certain embodiments, the “immune modulatory agent” is an anti-PD-1 or anti-PD-L1 antibody.

A “hybrid” cell refers to a cell having both antigen presenting capability and also expresses one or more specific antigens. In one embodiment, these hybrid cells are formed by fusing, in vitro, APCs with cells that are known to express the one or more antigens of interest. As used herein, the term “hybrid” cell and “fusion” cell are used interchangeably.

A “control” cell refers to a cell that does not express the same antigens as the population of antigen-expressing cells.

The term “culturing” refers to the in vitro propagation of cells or organisms on or in media of various kinds, it is understood that the descendants 30 of a cell grown in culture may not be completely identical (i.e., morphologically, genetically, or phenotypically) to the parent cell. By “expanded” is meant any proliferation or division of cells.

As used herein, the term “test sample” is a sample isolated, obtained or derived from a subject, e.g., a human subject.

The term “sufficient amount” or “amount sufficient to” means an amount sufficient to produce a desired effect. e.g., an amount sufficient to reduce the size of a tumor.

An “effective amount” is an amount sufficient to effect beneficial or desired results. An effective amount can be administered in one or more administrations, applications or dosages.

The term “therapeutically effective amount” is an amount that is effective to ameliorate a symptom of a disease. A therapeutically effective amount can be a “prophylactically effective amount” as prophylaxis can be considered therapy.

An “isolated” population of cells is “substantially free” of cells and materials with which it is associated in nature. By “substantially free” or “substantially pure” is meant at least 50% of the population are the desired cell type, preferably at least 70%, more preferably at least 80%, and even more preferably at least 90%. An “enriched” population of cells is at least 5% fused cells. Preferably, the enriched population contains at least 10%, more preferably at least 20%, and most preferably at least 25% fused cells.

The term “autogeneic”, or “autologous”, as used herein, indicates the origin of a cell. Thus, a cell being administered to an individual (the “recipient”) is autogeneic if the cell was derived from that individual (the “donor”) or a genetically identical individual (i.e., an identical twin of the individual). An autogeneic cell can also be a progeny of an autogeneic cell. The term also indicates that cells of different cell types are derived from the same donor or genetically identical donors. Thus, an effector cell and an antigen presenting cell are said to be autogeneic if they were derived from the same donor or from an individual genetically identical to the donor, or if they are progeny of cells derived from the same donor or from an individual genetically identical to the donor.

Similarly, the term “allogeneic”, as used herein, indicates the origin of a cell. Thus, a cell being administered to an individual (the “recipient”) is allogeneic if the cell was derived from an individual not genetically identical to the recipient. In particular, the term relates to non-identity in expressed MHC molecules. An allogeneic cell can also be a progeny of an allogeneic cell. The term also indicates that cells of different cell types are derived from genetically nonidentical donors, or if they are progeny of cells derived from genetically non-identical donors. For example, an APC is said to be allogeneic to an effector cell if they are derived from genetically non-identical donors.

A “subject” is a vertebrate, preferably a mammal, more preferably a human. Mammals include, but are not limited to, murines, simians, humans, farm animals, sport animals, and pets.

The terms “major histocompatibility complex” or “MHC” refers to a complex of genes encoding cell-surface molecules that are required for antigen presentation to immune effector cells such as T cells and for rapid graft rejection. In humans, the MHC complex is also known as the HLA complex. The proteins encoded by the MHC complex are known as “MHC molecules” and are classified into class I and class II MHC molecules. Class I MHC molecules include membrane heterodimeric proteins made up of an α chain encoded in the MHC associated noncovalently with β2-microglobulin. Class I MHC molecules are expressed by nearly all nucleated cells and have been shown to function in antigen presentation to CD8+ T cells. Class I molecules include HLA-A, -B, and -C in humans. Class II MHC molecules also include membrane heterodimeric proteins consisting of noncovalently associated and J3 chains. Class II MHCs are known to function in CD4+ T cells and, in humans, include HLA-DP, -DQ, and DR. The term “MHC restriction” refers to a characteristic of T cells that permits them to recognize antigen only after it is processed and the resulting antigenic peptides are displayed in association with either a class I or class II MHC molecule. Methods of identifying and comparing MHC are well known in the art and are described in Allen M. et al. (1994) Human Imm. 40:25-32; Santamaria P. et al. (1993) Human Imm. 37:39-50; and Hurley C. K. et al. (1997) Tissue Antigens 50:401-415.

The term “sequence motif” refers to a pattern present in a group of 15 molecules (e.g., amino acids or nucleotides). For instance, in one embodiment, the present invention provides for identification of a sequence motif among peptides present in an antigen. In this embodiment, a typical pattern may be identified by characteristic amino acid residues, such as hydrophobic, hydrophilic, basic, acidic, and the like.

The term “peptide” is used in its broadest sense to refer to a compound of two or more subunit amino acids, amino acid analogs, or peptidomimetics. The subunits may be linked by peptide bonds. In another embodiment, the subunit may be linked by other bonds, e.g. ester, ether, etc.

As used herein the term “amino acid” refers to either natural and/or 25 unnatural or synthetic amino acids, including glycine and both the D or L optical isomers, and amino acid analogs and peptidomimetics. A peptide of three or more amino acids is commonly called an oligopeptide if the peptide chain is short. If the peptide chain is long, the peptide is commonly called a polypeptide or a protein.

The term “aberrantly expressed” refers to polynucleotide sequences in a cell or tissue which are differentially expressed (either over-expressed or under-expressed) when compared to a different cell or tissue whether or not of the same tissue type, i.e., lung tissue versus lung cancer tissue.

An “antibody” is an immunoglobulin molecule capable of binding an antigen. As used herein, the term encompasses not only intact immunoglobulin molecules, but also anti-idiotypic antibodies, mutants, fragments, fusion proteins, humanized proteins and modifications of the immunoglobulin molecule that comprise an antigen recognition site of the required specificity.

An “antibody complex” is the combination of antibody and its binding partner or ligand.

A “native antigen” is a polypeptide, protein or a fragment containing an epitope, which induces an immune response in the subject.

By “interfering RNA” or “RNAi” or “interfering RNA sequence,” we refer to double-stranded RNA (i.e., duplex RNA) that targets (i.e., silences, reduces, or inhibits) expression of a target gene (Le., by mediating the degradation of mRNAs which are complementary to the sequence of the interfering RNA) when the interfering RNA is in the same cell as the target gene. Interfering RNA thus refers to the double stranded RNA formed by two complementary strands or by a single, self-complementary strand. Interfering RNA typically has substantial or complete identity to the target gene. The sequence of the interfering RNA can correspond to the full length target gene, or a subsequence thereof. Interfering RNA includes small-interfering “RNA” or “siRNA,” i.e., interfering RNA of about 15-60, 15-50, 15-50, or 15-40 (duplex) nucleotides in length, more typically about, 15-30, 15-25 or 19-25 (duplex) nucleotides in length, and is preferably about 20-24 or about 21-22 or 21-23 (duplex) nucleotides in length (e.g., each complementary sequence of the double stranded siRNA is 15-60, 15-50, 15-50, 15-40, 15-30, 15-25 or 19-25 nucleotides in length, preferably about 20-24 or about 21-22 or 21-23 nucleotides in length, and the double stranded siRNA is about 15-60, 15-50, 15-50, 15-40, 15-30, 15-25 or 19-25 preferably about 20-24 or about 21-22 or 21-23 base pairs in length). siRNA duplexes may comprise 3′ overhangs of about 1 to about 4 nucleotides, preferably of about 2 to about 3 nucleotides and 5′ phosphate termini, The siRNA can be chemically synthesized or may be encoded by a plasmid (e.g., transcribed as sequences that automatically fold into duplexes with hairpin loops). siRNA can also be generated by cleavage of longer dsRNA (e.g., dsRNA greater than about 25 nucleotides in length) with the E. coli RNase III or Dicer. These enzymes process the dsRNA into biologically active siRNA (see, e.g., Yang et al., PNAS USA 99: 9942-7 (2002); Calegari et al., PNAS USA 99: 14236 (2002); Byrom et al., Ambion TechNotes 10(1): 4-6 (2003); Kawasaki et al.; Nucleic Acids Res. 31:981-7 (2003); Knight and Bass, Science 2.93: 2269-71 (2001); and Robertson et al., J. Biol. Chem. 243: 82 (1968)). Preferably, dsRNA are at least 50 nucleotides to about 100, 200, 300, 400 or 500 nucleotides in length. A dsRNA may be as long as 1000, 1500, 2000, 5000 nucleotides in length or longer. The dsRNA can encode for an entire gene transcript or a partial gene transcript.

By “siRNA” we refer to a short inhibitory RNA that can be used to silence gene expression of a specific gene. The siRNA can be a short RNA hairpin (e.g. shRNA) that activates a cellular degradation pathway directed at mRNAs corresponding to the siRNA. Methods of designing specific siRNA molecules or shRNA molecules and administering them are known to a person skilled in the art. It is known in the art that efficient silencing is obtained with siRNA duplex complexes paired to have a two nucleotide 3′ overhang. Adding two thymidine nucleotides is thought to add nuclease resistance. A person skilled in the art will recognize that other nucleotides can also be added.

By “antisense nucleic acid” as used herein means a nucleotide sequence that is complementary to its target e.g. a tumor derived immune suppressive transcription product such as IL10. The nucleic acid can comprise DNA, RNA or a chemical analog, that binds to the messenger RNA produced by the target gene. Binding of the antisense nucleic acid prevents translation and thereby inhibits or reduces target protein expression. Antisense nucleic acid molecules may be chemically synthesized using naturally occurring nucleotides or variously modified nucleotides designed to increase the biological stability of the molecules or to increase the physical stability of the duplex formed with mRNA or the native gene e.g. phosphorothioate derivatives and acridine substituted nucleotides. The antisense sequences may be produced biologically using an expression vector introduced into cells in the form of a recombinant plasmid, phagemid or attenuated virus in which antisense sequences are produced under the control of a high efficiency regulatory region, the activity of which may be determined by the cell type into which the vector is introduced.

The term “isolated” means separated from constituents, cellular and otherwise, in which the polynucleotide, peptide, polypeptide, protein, antibody, or fragments thereof, are normally associated with in nature. As is apparent to those of skill in the art, a non-naturally occurring polynucleotide, peptide, polypeptide, protein, antibody, or fragments thereof, does not require “isolation” to distinguish it from its naturally occurring counterpart. In addition, a “concentrated”, “separated” or “diluted” polynucleotide, peptide, polypeptide, protein, antibody, or fragments thereof, is distinguishable from its naturally occurring counterpart in that the concentration or number of molecules per volume is greater than “concentrated” or less than “separated” than that of its naturally occurring counterpart. A polynucleotide, peptide, polypeptide, protein, antibody, or fragments thereof, which differs from the naturally occurring counterpart in its primary sequence or for example, by its glycosylation pattern, need not be present in its isolated form since it is distinguishable from its naturally occurring counterpart by its primary sequence, or alternatively, by another characteristic such as glycosylation pattern. Although not explicitly stated for each of the inventions disclosed herein, it is to be understood that all of the above embodiments for each of the compositions disclosed below and under the appropriate conditions, are provided by this invention. Thus, a non-naturally occurring polynucleotide is provided as a separate embodiment from the isolated naturally occurring polynucleotide. A protein produced in a bacterial cell is provided as a separate embodiment from the naturally occurring protein isolated from a eucaryotic cell in which it is produced in nature.

A “composition” is intended to mean a combination of active agent and another compound or composition, inert (for example, a detectable agent, carrier, solid support or label) or active, such as an adjuvant.

A “pharmaceutical composition” is intended to include the combination of an active agent with a carrier, inert or active, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin, REMINGTON'S PHARM. SCI, 15th Ed. (Mack Publ. Co., Easton (1975)).

As used herein, the term “inducing an immune response in a subject” is a term well understood in the art and intends that an increase of at least about 2-fold, more preferably at least about 5-fold, more preferably at least about 10-fold, more preferably at least about 100-fold, even more preferably at least about 500-fold, even more preferably at least about 1000-fold or more in an immune response to an antigen (or epitope) can be detected (measured), after introducing the antigen (or epitope) into the subject, relative to the immune response (if any) before introduction of the antigen (or epitope) into the subject. An immune response to an antigen (or epitope), includes, but is not limited to, production of an antigen-specific (or epitope-specific) antibody, and production of an immune cell expressing on its surface a molecule which specifically binds to an antigen (or epitope). Methods of determining whether an immune response to a given antigen (or epitope) has been induced are well known in the art. For example, antigen specific antibody can be detected using any of a variety of immunoassays known in the art, including, but not limited to, ELISA, wherein, for example, binding of an antibody in a sample to an immobilized antigen (or epitope) is detected with a detectably-labeled second antibody (e.g., enzyme-labeled mouse anti-human Ig antibody). Immune effector cells specific for the antigen can be detected any of a variety of assays known to those skilled in the art, including, but not limited to, FACS, or, in the case of CTLs, ⁵¹CR-release assays, or ³H-thymidine uptake assays.

By cellular proliferative and/or differentiative disorders we refer to cancer, e.g., carcinoma, sarcoma, metastatic disorders or hematopoietic neoplastic disorders, e.g., leukemias. A metastatic tumor can arise from a multitude of primary tumor types, including but not limited to those of prostate, colon, lung, breast and origin.

By “cancer”, “hyperproliferative” and “neoplastic” refer to cells having the capacity for autonomous growth, i,e., an abnormal state or condition characterized by rapidly proliferating cell growth. Hyperproliferative and neoplastic disease states may be categorized as pathologic, i.e., characterizing or constituting a disease state, or may be categorized as non-pathologic, i.e., a deviation from normal but not associated with a disease state. The term is meant to include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth. Examples of non-pathologic hyperproliferative cells include proliferation of cells associated with wound repair. The terms “cancer” or “neoplasms” include malignancies of the various organ systems, e.g., affecting the nervous system, lung, breast, thyroid, lymphoid, gastrointestinal, and genito-urinary tract, as well as adenocarcinomas, which include malignancies such as most colon cancers, renal-cell carcinoma, prostate cancer and/or testicular tumors, non-small cell carcinoma of the lung, cancer of the small intestine and cancer of the esophagus. The term “carcinoma” is art recognized and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. In some embodiments, the disease is renal carcinoma or melanoma. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures. The term “sarcoma” is art recognized and refers to malignant tumors of mesenchymal derivation.

As used herein, the term “cancer therapy” refers to a therapy useful in treating cancer. Examples of anti-cancer therapeutic agents include, but are not limited to, e.g., surgery, chemotherapeutic agents, immunotherapy, growth inhibitory agents, cytotoxic agents, agents used in radiation therapy, anti-angiogenesis agents, apoptotic agents, anti-tubulin agents, and other agents to treat cancer, such as anti-HER-2 antibodies (e.g., HERCEPTIN®), anti-CD20 antibodies, an epidermal growth factor receptor (EGFR) antagonist (e.g., a tyrosine kinase inhibitor), HER1/EGFR inhibitor (e.g., erlotinib (TARCEVA®)), platelet derived growth factor inhibitors (e.g., GLEEVEC™ (Imatinib Mesylate)), a COX-2 inhibitor (e.g., celecoxib), interferons, cytokines, antagonists (e.g., neutralizing antibodies) that bind to one or more of the following targets ErbB2, ErbB3, ErbB4, PDGFR-beta, BlyS, APRIL, BCMA or VEGF receptor(s), TRAIL/Apo2, and other bioactive and organic chemical agents, etc. Combinations thereof are also contemplated for use with the methods described herein.

A “chemotherapeutic agent” is a chemical compound useful in the treatment of cancer. Examples of chemotherapeutic agents include Erlotinib (TARCEVA®, Genentech/OSI Pharm.), Bortezomib (VELCADE®, Millennium Pharm.), Fulvestrant (FASLODEX®, Astrazeneca), Sutent (SU11248, Pfizer), Letrozole (FEMARA®, Novartis), Imatinib mesylate (GLEEVEC®, Novartis), PTK787/ZK 222584 (Novartis), Oxaliplatin (Eloxatin®, Sanofi), 5-FU (5-fluorouracil), Leucovorin, Rapamycin (Sirolimus, RAPAMUNE®, Wyeth), Lapatinib (GSK572016, GlaxoSmithKline), Lonafamib (SCH 66336), Sorafenib (BAY43-9006, Bayer Labs.), and Gefitinib (IRESSA®, Astrazeneca), AG1478, AG1571 (SU 5271; Sugen), alkylating agents such as Thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethiophosphoramide and trimethylomelamine; acetogenins (especially bullatacin and bullatacinone); a camptothecin (including the synthetic analogue topotecan); bryostatin; callystatin; CC-1065 (including its adozcicsin, carzcicsin and bizcicsin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimustine; antibiotics such as the enediyne antibiotics (e.g., calicheamicin, especially calicheamicin gammall and calicheamicin omegall (Angew Chem. Intl. Ed. Engl. (1994) 33:183-186); dynemicin, including dynemicin A; bisphosphonates, such as clodronate; an esperamicin; as well as neocarzinostatin chromophore and related chromoprotein enediyne antibiotic chromophores), aclacinomysins, actinomycin, anthramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, strcptonigrin, strcptozocin, tubcrcidin, ubenimcx, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacytidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosinc; arabinoside (“Ara-C”); cyclophosphamidc; thiotcpa; taxoids, e.g., TAXOL® paclitaxel (Bristol-Myers Squibb Oncology, Princeton, N.J.), ABRAXANE™ Cremophor-free, albumin-engineered nanoparticle formulation of paclitaxel (American Pharmaceutical Partners, Schaumberg, Ill.), and TAXOTERE® doxetaxel (Rhone-Poulenc Rorer, Antony, France); chloranbucil; GEMZAR® gemcitabine; 6-thioguanine; mercaptopurine; methotrexate; platinum analogs such as cisplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; CPT-11; topoisomerase inhibitor RFS 2000; difluoromethylomithine (DMFO); retinoids such as retinoic acid; capecitabine; and pharmaceutically acceptable salts, acids or derivatives of any of the above.

Also included in this definition of “chemotherapeutic agent” are: (i) anti-hormonal agents that act to regulate or inhibit hormone action on tumors such as anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY117018, onapristone, and FARESTON.toremifene; (ii) aromatase inhibitors that inhibit the enzyme aromatase, which regulates estrogen production in the adrenal glands, such as, for example, 4 (5)-imidazoles, aminoglutethimide, MEGASE® megestrol acetate, AROMASIN® exemestane, formestanie, fadrozole, RIVISOR® vorozole, FEMARA® letrozole, and ARIMIDEX® anastrozole; (iii) anti-androgens such as flutamide, nilutamide, bicalutamide, leuprolide, and goserelin; as well as troxacitabine (a 1,3-dioxolane nucleoside cytosine analog); (iv) aromatase inhibitors; (v) protein kinase inhibitors; (vi) lipid kinase inhibitors; (vii) antisense oligonucleotides, particularly those which inhibit expression of genes in signaling pathways implicated in abherant cell proliferation, such as, for example, PKC-alpha, Ralf and H-Ras; (viii) ribozymes such as a VEGF expression inhibitor (e.g., ANGIOZYME® ribozyme) and a HER2 expression inhibitor; (ix) vaccines such as gene therapy vaccines, for example, ALLOVECTIN.degree. vaccine, LEUVECTIN® vaccine, and VAXID® vaccine; PROLEUKIN® rIL-2; LURTOTECAN® topoisomerase 1 inhibitor; ABARELIX® rmRH; (x) anti-angiogenic agents such as bevacizumab (AVASTIN®, Genentech); and (xi) pharmaceutically acceptable salts, acids or derivatives of any of the above.

As used herein, “combination therapy” embraces administration of each agent or therapy in a sequential manner in a regiment that will provide beneficial effects of the combination and co-administration of these agents or therapies in a substantially simultaneous manner. Combination therapy also includes combinations where individual elements may be administered at different times and/or by different routes but which act in combination to provide a beneficial effect by co-action or pharmacokinetic and pharmacodynamics effect of each agent or tumor treatment approaches of the combination therapy. For example, the agents or therapies may be administered simultaneously, sequentially, or in a treatment regimen in a predetermined order.

A “cancer vaccine,” as used herein is a composition that stimulates an immune response in a subject against a cancer. Cancer vaccines typically consist of a source of cancer-associated material or cells (antigen) that may be autologous (from self) or allogenic (from others) to the subject, along with other components (e.g., adjuvants) to further stimulate and boost the immune response against the antigen. Cancer vaccines can result in stimulating the immune system of the subject to produce antibodies to one or several specific antigens, and/or to produce killer T cells to attack cancer cells that have those antigens.

By substantially free of endotoxin is meant that there is less endotoxin per dose of cell fusions than is allowed by the FDA for a biologic, which is a total endotoxin of 5 EU/kg body weight per day.

By substantially free for Mycoplasma and microbial contamination is meant as negative readings for the generally accepted tests know to those skilled in the art. For example, mycoplasm contamination is determined by subculturing a cell sample in broth medium and distributed over agar plates on day 1, 3, 7, and 14 at 37° C. with appropriate positive and negative controls. The product sample appearance is compared microscopically, at 100×, to that of the positive and negative control. Additionally, inoculation of an indicator cell culture is incubated for 3 and 5 days and examined at 600× for the presence of mycoplasmas by epifluorescence microscopy using a DNA-binding fluorochrome. The product is considered satisfactory if the agar and/or the broth media procedure and the indicator cell culture procedure show no evidence of Mycoplasma contamination.

The sterility test to establish that the product is free of microbial contamination is based on the U.S. Pharmacopedia Direct Transfer Method. This procedure requires that a pre-harvest medium effluent and a pre-concentrated sample be inoculated into a tube containing tryptic soy broth media and fluid thioglycollate media. These tubes are observed periodically for a cloudy appearance (turpidity) for a 14 day incubation. A cloudy appearance on any day in either medium indicate contamination, with a clear appearance (no growth) testing substantially free of contamination.

### EXAMPLES

### Example 1: General Methods

Cell culture. Human A549/KRAS(G12S), H460/KRAS(Q61H) and H1975/EGFR(L858R/T790M) NSCLC cells (ATCC) were grown in RPMI1640 medium supplemented with 10% heat-inactivated fetal bovine serum (HI-FBS), 100 μg/ml streptomycin, 100 units/ml penicillin and 2 mM L-glutamine. Authentication of the cells was performed by short tandem repeat (STR) analysis. Cells were transfected with lentiviral vectors to stably express a scrambled control shRNA (CshRNA; Sigma), a MUC1 shRNA (MUC1shRNA; Sigma), a NF-κB p65 shRNA (Sigma), MUC1-C or MUC1-C (AQA) (27, 8, 49). Cells were treated with the IκB inhibitor BAY-11-7085 (Sigma) or DMSO as the vehicle control. Cells were also treated with empty nanoparticles (NPs) or GO-203/NPs (39).

Quantitative real-time, reverse transcriptase PCR (qRT-PCR). Whole cell RNA was isolated using the RNeasy mini kit (Qiagen). The High Capacity cDNA Reverse Transcription kit (Life Technologies) was used to synthesize cDNAs from 2 μg RNA. The SYBR green qPCR assay kit and the ABI Prism Sequence Detector (Applied Biosystems) were used to amplify the cDNAs. Primers used are listed in Table 1.

Immunoblot analysis. Whole cell lysates were prepared in NP-40 lysis buffer and immunoblotted with antibodies against MUC1-C (LabVision), PD-L1 (Cell Signaling Technology) and β-actin (Sigma). Horseradish peroxidase secondary antibodies and enhanced chemiluminescence (GE Healthcare) were used for the detection of immune complexes.

Promoter-reporter assays. Cells were transfected with 1.5 μg of PD-L1 promoter-luciferase reporter (pPD-L1-Luc) or control vector (Active Motif) and SV-40-Renilla-Luc with Superfect (Qiagen). After 48 h, the cells were lysed in passive lysis buffer. Lysates were analyzed using the Dual-Luciferase assay kit (Promega).

Chromatin immunoprecipitation (ChIP) assays. Soluble chromatin was isolated from 3×106 cells and immunoprecipitated with anti-NF-κB p65 (Santa Cruz Biotechnology) or a control IgG as described (44). In re-ChIP experiments, NF-κB complexes obtained were reimmunoprecipitated with anti-MUC1-C (NeoMarkers) or a control IgG. qPCR analyses were performed using the SYBR green kit and the ABI Prism 7000 Sequence Detector (Applied Biosystems). Primers used for the PD-L1, TLR9 and IFNG promoters and GAPDH as a control are listed in Table 2. Relative fold enrichment was calculated as described (62).

NSCLC tumor xenograft studies. H460 cells (5×106) were injected subcutaneously in the flank of six-week old female NCR nu/nu mice. After reaching a tumor size of ˜150 mm3, mice were pair-matched in two groups and treated with empty NPs or 15 mg/kg GO-203/NPs. The formula V=L×W2/2, where L and W are the larger and smaller diameters, respectively, was used to calculate tumor volumes.

Statistics. Statistical significance was determined using the Student's t-test.

Bioinformatic analyses. NSCLC clinical datasets were obtained from The Cancer Genome Atlas (TCGA) and Gene Expression Omnibus (GEO) under the accession number (GSE72094) (34, 49, 63). GSE72084 microarray gene expression data were normalized with IRON as described (64). TCGA data were obtained from Firehose by using RTCGAToolbox (65). Log 2 expression values of MUC1, IFN-γ, TLR9 and MCP-1/CCL2 from both datasets were assessed for correlation using Spearman's coefficient. The prognostic value of TLR9, IFN-γ, MCP-1/CCL2 and CSF2/GMCSF expression in NSCLC dataset (GSE19188) was performed as previously described (Gyorffy, 2013, #10658; Goodwin, 2014, #10659). Expression values of TLR9, IFN-γ, MCP-1/CCL2 and CSF2/GMCSF were averaged and NSCLC patients were divided by the median expression. The Kaplan-Meier survival probability plot with the hazard ratio (95% confidence interval) and long rank p value was calculated and plotted in R.

TABLE 1
qPCR Primer Pairs

| Name | Forward | Reverse |
|---|---|---|
| MUC1 | 5′-GAAAGAACTACGGGCAGCTGG-3′ | 5′-CAAGTTGGCAGAAGTGGCTGC-3′ |
| PD-L1 | 5′-CCTACTGGCATTTGCTGAACGCAT- | 5′-CAATAGACAATTAGTGCAGCCAGGTC- |
| | 3′ | 3′ |
| TLR9 | 5′-CAACAACCTCACTGTGGTGC-3′ | 5′-GAGTGAGCGGAAGAAGATGC-3′ |
| IFNγ | 5′-CTAATTATTCGGTAACTGACTTGA- | 5′-ACAGTTCAGCCATCACTTGGA-3′ |
| | 3′ | |
| TIM-3 | 5′-GACTTCACTGCAGCCTTTCC-3′ | 5′-GATCCCTGCTCCGATGTAGA-3′ |
| CTLA- | 5′-CTACCTGGGCATAGGCAACG-3′ | 5′-CCCCGAACTAACTGCTGCAA-3′ |
| 4 | | |
| LAG-3 | 5′-CAGGAACAGCAGCTCAATGC-3′ | 5′-AGGGATCCAGGTGACCCAAA-3′ |
| PD-1 | 5′-CAACACATCGGAGAGCTTCGT-3′ | 5′-GGAAGGCGGCCAGCTT-3′ |
| PD-L2 | 5′-GTACATAATAGAGCATGGCAGCA-3′ | 5′-CCACCTTTTGCAAACTGGCTGT-3′ |
| TLR7 | 5′-CTCCCTGGATCTGTACACCTGTGAG- | 5′-CTCCCACAGAGCCTTTTCCGGAGCT- |
| | 3′ | 3′ |
| MCP-1 | 5′-TCCTCCTTCTCTCTGTCCATTA-3′ | 5′-CCCAGTGCTTCTGCCTATAC-3′ |
| GM- | 5′-CTGCTGAGATGGTAAGTGAGAG-3′ | 5′-CATCTTACCTGGAGGTCAAACA-3′ |
| CSF | | |

TABLE 2
Promoter ChIP qPCR Primer Pairs

| Name | Forward | Reverse |
|---|---|---|
| PD-L1 | 5′-CAAGGTGCGTTCAGATGTTG-3′ | 5′-GGCGTTGGACTTTCCTGA-3′ |
| TLR9 | 5′-GTGGACCCAGCAGAACTTG-3′ | 5′-CTTCCCACTCTCCTTCTGATCTA-3′ |
| IFNγ | 5′-CAAAGGACCCAAGGAGTCTAAAG- | 5′-ACAGATAGGCAGGGATGATAGT-3′ |
| | 3′ | |
| GAPDH | 5′-TACTAGCGGTTTTACGGGCG-3′ | 5′-TCGAACAGGAGGAGCAGAGAGCGA-3′ |

### Example 2: MUC1-C Drives PD-L1 Expression NSCLC Cells

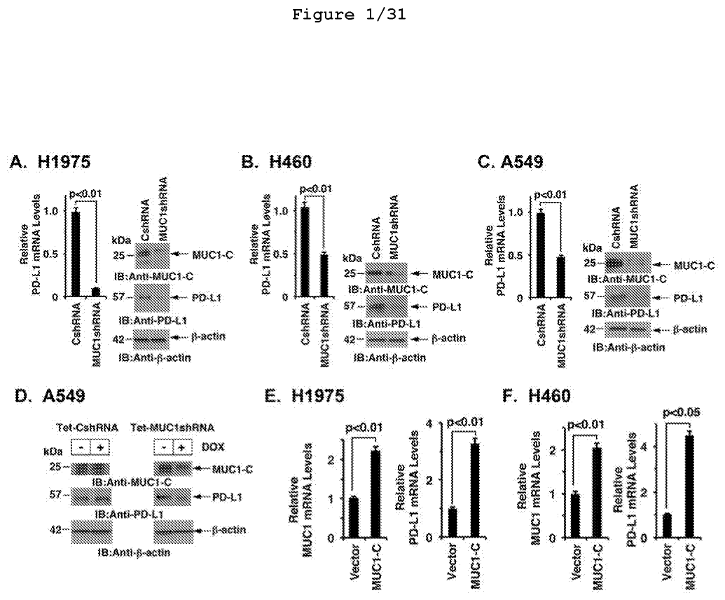

NSCLC cells driven by mutant EGFR activate the PD-1/PD-L1 pathway. (6) We have also shown that targeting MUC1-C in NSCLC cells is associated with suppression of EGFR(L858R/T790M) activation (27), invoking the possibility that MUC1-C could contribute to PD-L1 expression. Indeed, silencing MUC1-C in H1975/EGFR(L858R/T790M) cells resulted in suppression of PD-L1 mRNA (FIG. 1A, left) and protein (FIG. 1A, right). Previous work has also shown that MUC1-C confers EMT and self-renewal capacity in A549/KRAS(G12S) and H460/KRAS(Q61H) cells (28). To extend these observations to KRAS mutant NSCLC cells, we silenced MUC1-C in H460 cells and also found downregulation of PD-L1 expression (FIG. 1B, left and right). Similar results were obtained with A549 cells (FIG. 1C, left and right), indicating that stable silencing of MUC1-C in NSCLC cells decreases PD-L1 expression. To confirm these findings, we established A549 cells transduced to express a tetracycline-inducible tet-on control shRNA (tet-CshRNA) or MUC1 shRNA (tet-MUC1shRNA). Treatment of A549/tet-CshRNA cells with doxycycline (DOX) for 7 days had no apparent effect on MUC1-C or PD-L1 expression (FIG. 1D, left). By contrast, treatment of A549/tet-MUC1shRNA cells resulted in suppression of MUC1-C, as well as PD-L1, expression (FIG. 1D, right). In further support of a MUC1-PD-L1 pathway, enforced overexpression of MUC1-C in H1975 (FIG. 1E) and H460 (FIG. 1F) cells was associated with upregulation of PD-L1 expression, indicating that MUC1-C is sufficient for this response. These results supported the premise that MUC1-C is necessary for PD-L1 expression in mutant EGFR and KRAS NSCLC cells.

### Example 3: Targeting the MUC1-C Cytoplasmic Domain Downregulates PD-L1 Expression

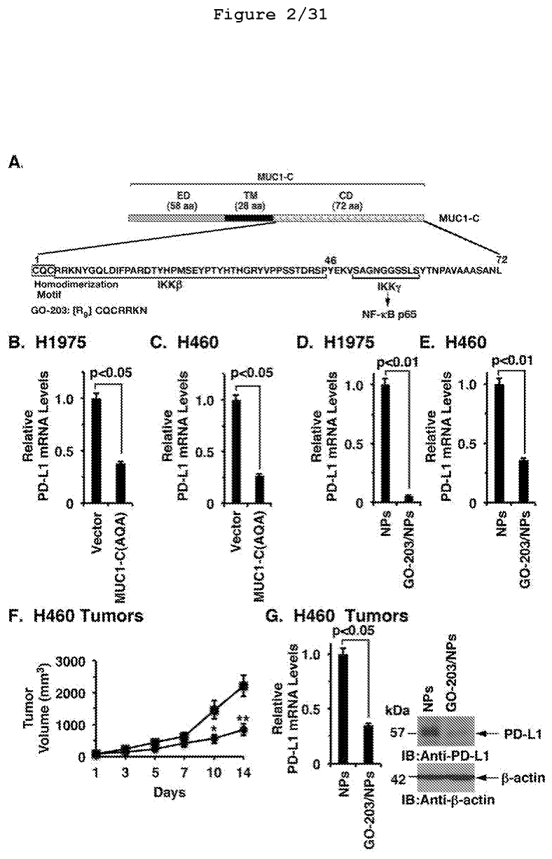

MUC1-C includes a 58 aa extracellular domain, a 28 aa transmembrane domain and a 72 aa cytoplasmic domain (FIG. 2A). The MUC1-C cytoplasmic domain contains a CQC motif that is necessary for MUC1-C homodimerization, nuclear localization and function as an oncoprotein (FIG. 2A) (23, 38). In this respect, expression of a MUC1-C (CQC→AQA) mutant in NSCLC cells blocks anchorage-independent growth and tumorigenicity, supporting a dominant-negative effect (27, 28, 38). We also found that stable expression of the MUC1-C (CQC→AQA) mutant in H1975 (FIG. 2B) and H460 (FIG. 2C) cells results in suppression of PD-L1 expression, further indicating that the MUC1-C cytoplasmic domain confers this response. Targeting the MUC1-C CQC motif with the cell-penetrating peptide, GO-203, blocks MUC1-C homodimerization and signaling in NSCLC cells (27, 28, 37). GO-203 has been recently formulated in polymeric nanoparticles (NPs) for more effective intracellular delivery to cancer cells growing in mouse models (39). Treatment of H1975 and H460 cells with GO-203/NPs, but not empty NPs, was associated with downregulation of PD-L1 expression (FIGS. 2D and 2E). Moreover, GO-203/NP treatment of mice bearing H460 tumor xenografts was associated with inhibition of growth (FIG. 2F) and suppression of PD-L1 mRNA and protein (FIG. 2G, left and right). These findings demonstrate that MUC1-C is a target for downregulating PD-L1 in NSCLC cells.

### Example 4: MUC1-C Drives PD-L1 Transcription by an NF-κB p65-Dependent Mechanism

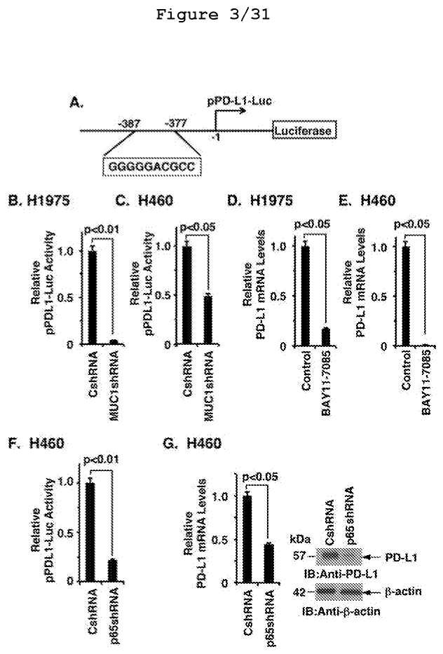

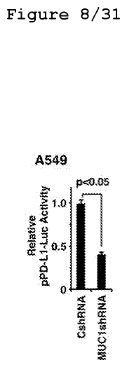

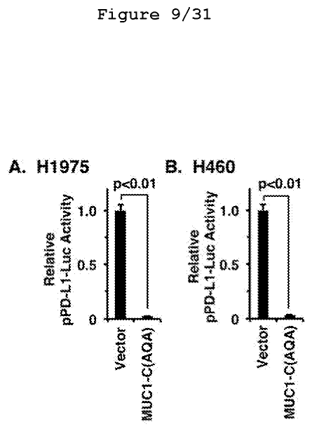

To define the mechanism by which MUC1-C drives PD-L1 expression, we first transfected H1975/CshRNA and H1975/MUC1shRNA cells with a PD-L1 promoter-luciferase reporter (pPD-L1-Luc) (FIG. 3A). The experiment revealed that silencing MUC1-C decreases pPD-L1-Luc activity (FIG. 3B). Similar results were obtained in H460 (FIG. 3C) and A549 (FIG. 8) cells, indicating that MUC1-C induces PD-L1 transcription. Along these lines, expression of the MUC1-C (CQC→AQA) mutant in H1975 (FIG. 9A) and H460 (Fig. S9B) cells also suppressed PD-L1 promoter activity. MUC1-C activates the inflammatory TAK1→IKK→NF-κB p65 pathway (32-34). Accordingly, we asked if treatment of NSCLC cells with BAY-11-7085, an irreversible inhibitor of IκBα phosphorylation, affects PD-L1 expression. Using this approach, we found that inhibiting the NF-κB pathway in H1975 (FIG. 3D) and H460 (FIG. 3E) cells suppresses PD-L1 expression. In concert with these findings, silencing NF-κB p65 in H460 cells was associated with downregulation of PD-L1 transcription (FIG. 3F) and PD-L1 mRNA and protein (FIG. 3G, left and right).

### Example 5: MUC1-C/NF-κB p65 Complexes Occupy the PD-L1 Promoter

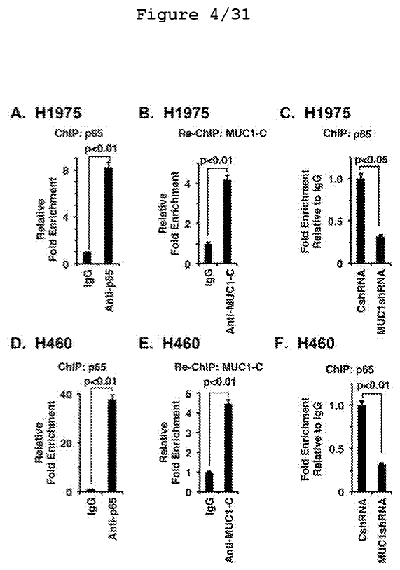

A potential NF-κB binding site (GGGGGACGCC) is located in the PD-L1 promoter at position −377 to −387 upstream to the transcription start site (FIG. 3A). ChIP analysis of H1975 cell chromatin demonstrated occupancy of the PD-L1 promoter by NF-κB p65 (FIG. 4A). Moreover and in concert with the finding that MUC1-C binds directly to NF-κB p65 (33), re-ChIP studies demonstrated that MUC1-C forms a complex with NF-κB p65 on the PD-L1 promoter (FIG. 4B). We also found that silencing MUC1-C decreases NF-κB p65 occupancy (FIG. 4C), consistent with previous studies on promoters of other NF-κB target genes, including MUC1 itself (33). Similar results were obtained in experiments with H460 cells; that is, (i) occupancy of the PD-L1 promoter by NF-κB p65/MUC1-C complexes (FIGS. 4D and 4E), and (ii) silencing MUC1-C decreases occupancy by NF-κB p65 (FIG. 4F).

### Example 6: Targeting MUC1-C Induces TLR7 Expression by an NF-κB p65-Mediated Mechanism

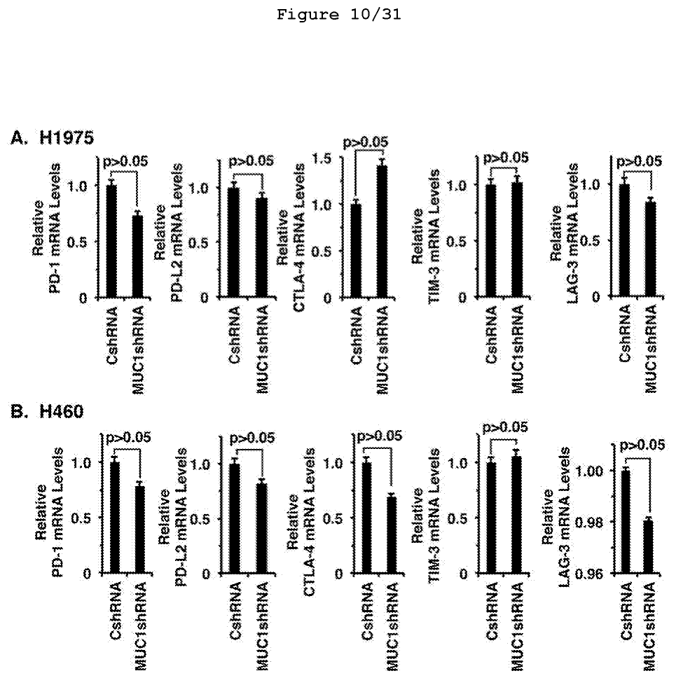

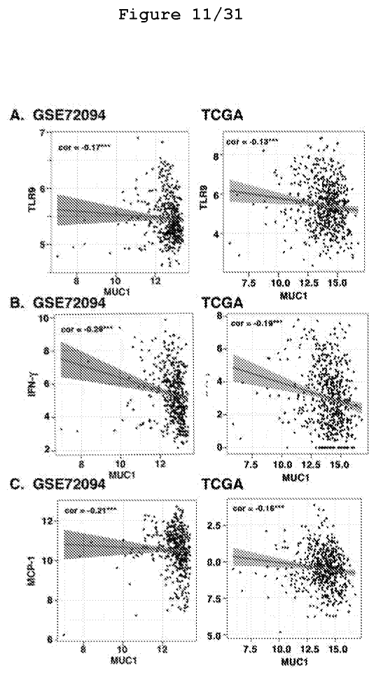

The above findings that MUC1-C induces PD-L1 expression through NF-κB p65 supported the notion that MUC1-C could also regulate other genes involved in immune responses. However, we found that targeting MUC1-C in H1975 (FIG. 10A) and H460 (FIG. 10B) cells had no significant effect on PD-1, PD-L2, CTLA-4, TIM-3 and LAG-3 expression, indicating that the above findings are selective for PD-L1. We therefore investigated the role of MUC1-C in regulation of the toll-like receptor 7 (TLR7) gene, which is constitutively expressed in NSCLC cells, activates the NF-κB pathway and confers chemoresistance and poor survival (40-42). In concert with the demonstration that the TLR7 promoter is activated by NF-κB p65 (Fig. S4A) (43), we found that targeting MUC1-C results in marked downregulation of TLR7 mRNA levels (FIG. 11B). Moreover targeting the NF-κB pathway with BAY-11-7085 (FIG. 11C) or silencing NF-κB p65 (FIG. 1D) was associated with decreases in TLR7 mRNA levels, indicating that, like PD-L1, the MUC1-C→NF-κB p65 pathway induces TLR7 in NSCLC cells.

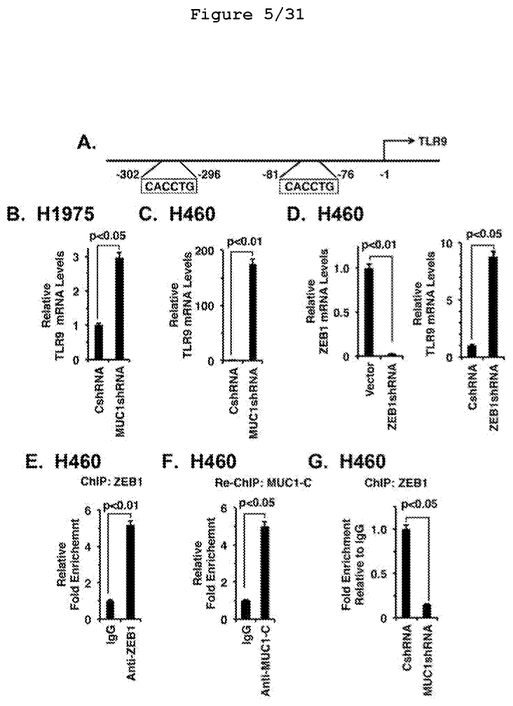

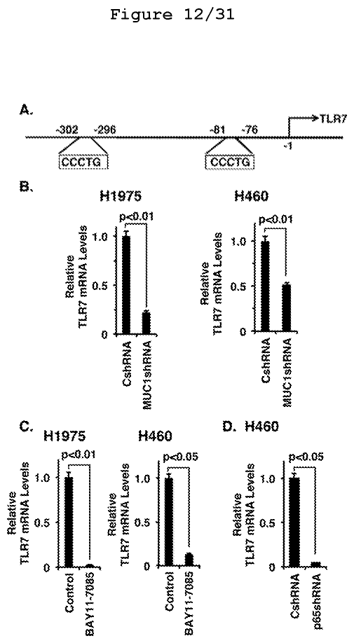

Targeting MUC1-C derepresses ZEB1-suppressed immune-related genes. The MUC1-C→NF-κB p65 pathway also activates the ZEB1 gene, which encodes the EMT-inducing transcription factor (44). In turn, MUC1-C interacts with ZEB1, represses miR-200c and induces EMT (44). Based on these findings, we reasoned that the MUC1-C→NF-κB→ZEB1 pathway might link EMT with the suppression of certain immune-related genes. Accordingly, we identified genes that are induced in response to silencing both MUC1-C and ZEB1. As one candidate, we studied the TLR9 gene, which encodes the innate TLR9 receptor, is downregulated by NF-κB signaling (45) and has two GC-rich E-boxes as potential binding sites for MUC1-C/ZEB1 complexes (FIG. 5A). Notably, targeting MUC1-C (FIGS. 5B and 5C) was associated with upregulation of TLR9 mRNA level. Moreover, silencing ZEB1 also resulted in induction of TLR9 expression (FIG. 5D). ChIP studies further demonstrated that ZEB1 occupies the TLR9 promoter in a complex with MUC1-C (FIGS. 5E and 5F). Silencing MUC1-C also decreased ZEB1 occupancy on the TLR9 promoter (FIG. 5G). Consistent with these results, analysis of TCGA and RNA-seq datasets demonstrated that MUC1 correlates negatively with TLR9 expression in NSCLCs (FIG. 12A, left and right).

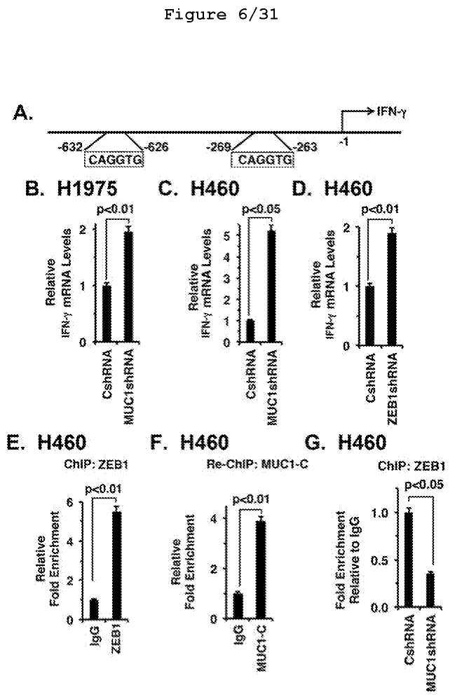

We also studied effects of targeting MUC1-C on the gene, which encodes IFN-γ and contains E-boxes in its promoter (FIG. 6A). As shown for TLR9, silencing MUC1-C in H1975 and H460 cells induced IFN-γ expression (FIGS. 6B and 6C). In addition, silencing ZEB1 was associated with increases in IFN-γ mRNA levels (FIG. 6D). ChIP experiments further demonstrated that (i) ZEB1 occupies the IFNG promoter in a complex with MUC1-C (FIGS. 6E and 6F) and (ii) MUC1-C promotes ZEB1 occupancy (FIG. 6G), supporting the notion that MUC1-C suppresses IFNγ activation by a ZEB1-mediated mechanism. In this respect, MUC1 correlated negatively with IFN-γ expression in NSCLCs (FIG. 12B, left and right).

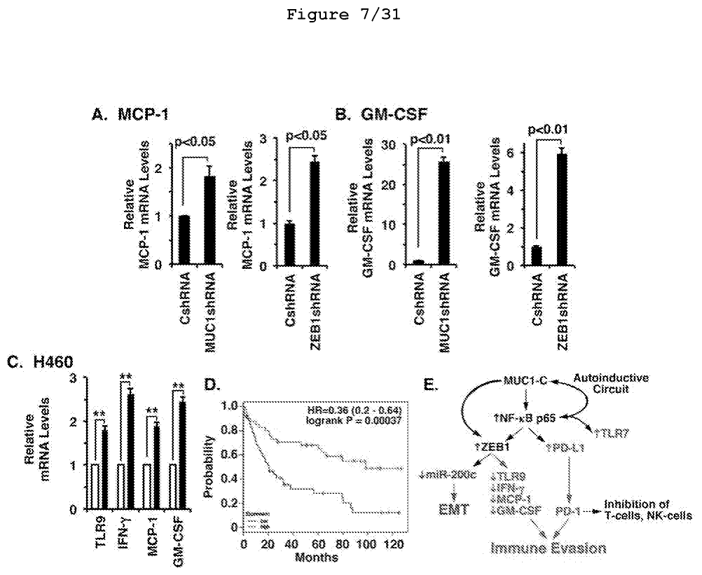

Along these same lines, we found that targeting MUC1-C and ZEB1 was associated with upregulation of (i) MCP-1/CCL2, a key chemokine that regulates the migration and infiltration of monocytes/macrophages (46) (FIG. 7A), and (ii) GM-CSF, a key hematopoietic growth factor and immune modulator (47) (FIG. 7B), supporting the notion that MUC1-C/ZEB1 complexes contribute to repression of the MCP-1 and GM-CSF gene. In concert with these findings, (i) the MCP-1 gene intron 1 region has 4 putative ZEB1 binding sites (CAGCTG) at +294 to +300, +328 to +334, +400 to +406 and +616 to +622 and (ii) the GM-CSF promoter contains a potential E-box (CACGTG) at −1097 to −1103 relative to their transcription start sites. Additionally, like TLR9 and IFN-γ, we found that MUC1 negatively correlates with MCP-1 in NSCLCs (FIG. 12C, left and right). In contrast, a negative correlation between MUC1 and GM-CSF was not statistically significant. Nonetheless and in concert with these results, treatment of H460 tumors with GO-203/NPs was associated with increases in TLR9, IFN-γ, MCP-1 and GM-CSF expression (FIG. 7C), indicating that targeting MUC1-C in vivo reverses this program of immune evasion. We also found that low levels of TLR9, IFN-γ, MCP-1 and GM-CSF in NSCLCs are associated with significant decreases in overall survival (FIG. 7D), further indicating that the MUC1-C→NF-κB→ZEB1 pathway suppresses multiple immune-related genes and thereby confers poor clinical outcomes.

### Example 7: General Methods: In Vivo Mouse Model Studies

Cell culture. Mouse LLC cells stably transfected with full length MUC1 (gift from Dr. Stephen Tomlinson, Medical University of South Carolina, Charleston, S.C.) were grown in DMEM medium supplemented with 10% heat-inactivated fetal bovine serum (HI-FBS), 100 μg/ml streptomycin, 100 units/ml penicillin and 2 mM L-glutamine. LLC cells were transfected with lentiviral vectors to stably express a control vector or MUC1-C. Geneticin (LLC/MUC1) and hygromycin (LLC/Vector, LLC/MUC1-C) were used to maintain a selection pressure. Authentication of the cells was performed by short tandem repeat (STR) analysis. Mycoplasma levels were measured monthly using the MycoAlert Mycoplasma Detection Kit (Lonza, Rockland, Mass., USA).

Immunoblot analysis. Whole cell lysates were prepared in NP-40 lysis buffer and immunoblotted with (i) anti-MUC1-C (ThermoFisher Scientific, Waltham, Mass., USA; Cat. # HM-1630-P1) and an anti-Armenian hamster secondary antibody (Abcam, Cambridge, Mass., USA; Cat. # ab5745), (ii) anti-PD-L1 (R&D Systems, Minneapolis, Minn., USA; Cat. # AF1019) and an anti-goat secondary antibody (Santa Cruz Biotechnology, Dallas, Tex., USA; Cat. # SC-2028, 1:3000 dilution) and (iii) anti-β-actin (Sigma, St. Louis, Mo., USA; Cat. A5316) and an anti-mouse secondary antibody (GE Healthcare Life Sciences, Pittsburgh, Pa., USA; Cat. # NA931). Horseradish peroxidase secondary antibodies and enhanced chemiluminescence (GE Healthcare Life Sciences) were used for the detection of immune complexes. Immunoblot results were each confirmed with two other analysis.

Quantitative real-time, reverse transcriptase PCR (qRT-PCR). The RNeasy mini kit (Qiagen, Germantown, Md., USA) was used to isolate whole cell RNA. The High Capacity cDNA Reverse Transcription kit (Life Technologies, Carlsbad, Calif., USA) was used to synthesize cDNAs from 2 μg RNA. The GAPDH gene was used as an internal control. The SYBR green qPCR assay kit and the ABI Prism Sequence Detector (Applied Biosystems, Foster City, Calif., USA) were used to amplify the cDNAs.

Animal studies. LLC/MUC1 cells (106 cells) were injected subcutaneously in the flank of six-week old MUC1.Tg mice. The mice were grouped and treated with control NPs or 15 mg/kg GO-203/NPs once a week for 2 weeks. At the end of the treatment, tumor tissues were harvested and processed for multi-parameter staining.

Flow cytometry. To generate cell suspensions, tumors were cut into small pieces, and further dissociated in RPMI-1640 buffer containing 5% FBS, 100 IU/ml collagenase type IV (Invitrogen, Carlsbad, Calif., USA), and 50 μg/ml DNAse I (Roche, Basel, Switzerland) for 45 min at 37° C. After incubation, cells were treated with red blood cell lysis buffer and filtered through a 70 μm cell strainer. After centrifugation, cell pellets were resuspended in 1×PBS/2% FBS. Approximately 0.5-1×106 cells were stained for surface markers in 1×PBS/2% FBS for 15 min at 4° C. Intracellular staining was performed for granzyme B using the Foxp3 staining buffer set (eBioscience, Santa Clara, Calif., USA). For intracellular cytokine detection assays, immune cells from tumors were obtained after Ficoll gradient separation. Cells (1×106) were cultured with PMA (50 ng) and ionomycin (500 ng) for 6 h at 37° C. GolgiPlug (BD Pharmingen, San Jose, Calif., USA) and FITC-conjugated CD107α (Biolegend, San Diego, Calif., USA; 1D4B) were added for the last 5 h of culture. The Cytofix/Cytoperm kit (BD Biosciences, San Jose, Calif.) was used for intracellular cytokine staining. Briefly, cells were washed with 1×PBS after harvesting, then stained for surface markers including CD8, and CD3, followed by intracellular staining with PE-conjugated anti-IFN-γ and Pacific blue anti-granzyme B or respective isotype-matched mAbs. In all stained samples, dead cells were excluded using Live/Dead Fixable Dead Cell staining kit (Invitrogen). Cells were acquired on the LSR Fortessa (BD Biosciences) and analyzed with FlowJo software (Tree Star, Ashland, Oreg., USA).

The following antibodies were utilized for staining in FACS analyses: FITC/AF488-conjugated mAbs to CD45 (30-F11), PE-conjugated mAbs to IFN-γ (XMG1.2), PerCP-conjugated mAbs to Nkp46 (29A1.4), CD45 (30-F11), APC/AF647-conjugated mAbs to PD-L1 (10F.9G2), Foxp3 (FJK-16s), Rat IgG (eBR2a), Pacific Blue/BV421-conjugated mAbs to Ki67 (16A8), granzyme B (GB11), CD4 (RM4-5), Rat IgG (eBRG1), PE-Cy7-conjugated mAbs to CD3 (17A2), CD62L (MEL-14), PD-L1 (10F.9G2), CD69 (H1.2F3), Rat IgG (RTK2758), APC-Cy7-conjugated mAbs to CD 4 (GK1.5), Alexa-Fluor 700-conjugated mAbs to CD8 (53-6.7), Rat IgG (RTK4530), were purchased from BD Biosciences, Biolegend or eBioscience.

CTL assays. The day before mice sacrifice, LLC/MUC1 cells (6×103 per well) were plated in 96-well plates and incubated overnight. Lymph nodes were harvested, digested with ACK lysis buffer (GIBCO, Waltham, Mass., USA) and rinsed with PBS. Cells (effector cells) were incubated with LLC/MUC1 cells (target cells) at different ratios in 96 well-plates for 6 h. The percentage of cytotoxicity was determined by measuring LDH release following the manufacturer's recommendations (CytoTox 96® Non-Radioactive Cytotoxicity Assay; Promega, Madison, Wis., USA) and calculated using the formula: ((Experimental-Effector spontaneous−Target spontaneous)/(Target maximum−Target spontaneous)×100.

Bioinformatic analysis. Clinical data of NSCLC patients was obtained from cBioPortal TCGA datasets (67). Correlations between MUC1 and CD8 (CD8A/B), IFNG and GZMB expression were assessed using Spearman's coefficient. The prognostic value of CD8 and IFNG in NSCLC patients was performed as described (68). Multiple probe set IDs were averaged for each sample. Patients were divided by the median expression. The Kaplan-Meier survival probability plot with the hazard ratio (95% confidence interval) and log-rank P-value were calculated and plotted in R.

Statistical analysis. Normal distribution of the data was confirmed using the Shapiro-Wilk test. The Student's t-test was used to determine statistical significance (GraphPad Software Inc, LaJolla, Calif., USA).

### Example 8: Effects of Targeting MUC1-C in an Immuno-Competent MUC1 Transgenic (MUC1.Tg)

We show that Lewis Lung Carcinoma cells expressing MUC1-C (LLC/MUC1) exhibit upregulation of PD-L1 and suppression of interferon-γ (IFN-γ). In studies of LLC/MUC1 cells growing in vitro and as tumors in MUC1.Tg mice, treatment with the MUC1-C inhibitor, GO-203, was associated with the downregulation of PD-L1 and induction of IFN-γ. The results further demonstrate that targeting MUC1-C results in enhanced activation and effector function of CD8+ tumor infiltrating lymphocytes (TILs) as evidenced by increased expression of the activation marker CD69, the degranulation marker CD107α and granzyme B. Notably, targeting MUC1-C was also associated with marked increases in TIL-mediated killing of LLC/MUC1 cells. Analysis of gene expression datasets further showed that overexpression of MUC1 in NSCLCs correlates negatively with CD8, IFNG and GZMB, and that decreases in CD8 and IFNG are associated with poor clinical outcomes. These findings in LLC/MUC1 tumors and in NSCLCs indicate that MUC1-C→PD-L1 signaling promotes the suppression of CD8+ T-cell activation and that MUC1-C is a potential target for reprogramming of the tumor microenvironment.

Few mechanistic insights are available regarding how NSCLCs evade immune recognition and destruction. In this regard, the present study evaluated how mucin 1 (MUC1) expression in tumor cells contributes to evasion of immune recognition and destruction in a model of NSCLC.

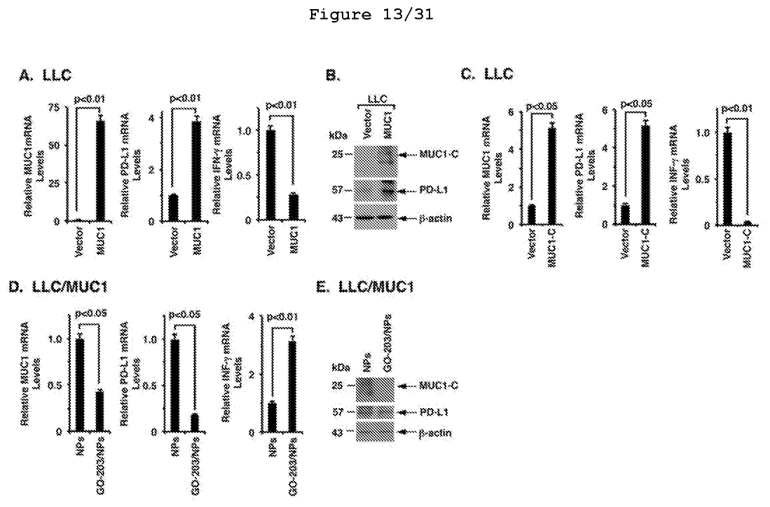

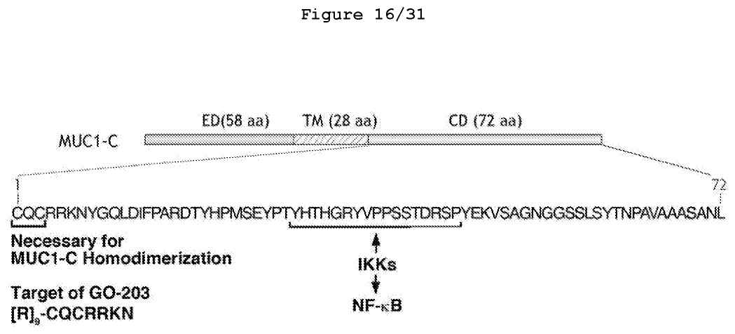

We studied Lewis Lung Carcinoma (LLC) cells stably expressing human MUC1 (LLC/MUC1). As expected, LLC/MUC1 cells exhibited high levels of MUC1 mRNA relative to that in control LLC cells expressing an empty vector (LLC/vector) (FIG. 13A). We also found that MUC1 increases PD-L1 and suppresses IFN-γ mRNA levels (FIG. 13A). MUC1 is a heterodimeric complex consisting of an extracellular N-terminal subunit (MUC1-N) and a transmembrane C-terminal subunit (MUC1-C) that functions as an oncoprotein (69, 22, 70). MUC1-C (20-25 kDa) includes a short extracellular domain, a transmembrane domain and an intrinsically disordered 72 amino acid cytoplasmic domain, which interacts with diverse kinases and effectors, such as NF-κB p65, that have been linked to inflammation and transformation (FIG. 16) (69, 22, 70). In this context, MUC1-C expression in LLC/MUC1 cells was associated with upregulation of PD-L1 protein (FIG. 13B). Additionally, overexpression of human MUC1-C in LLC cells was associated with induction of PD-L1 and suppression of IFN-γ (FIG. 13C), indicating that MUC1-C and not MUC1-N is sufficient for these responses. The MUC1-C cytoplasmic domain contains a CQC motif that is necessary for MUC1-C homodimerization and thereby function in signaling at the cell membrane and in the nucleus (FIG. 16) (69, 22, 70). Accordingly, we developed the GO-203 peptide inhibitor to target the CQC motif and block MUC1-C homodimerization and function (FIG. 16) (36,37). GO-203 has also been formulated in polymeric nanoparticles (GO-203/NPs) for sustained delivery in mouse tumor models (39). In concert with these and the above findings, treatment of LLC/MUC1 cells with GO-203/NPs, but not empty NPs, was associated with the (i) downregulation of MUC1 and PDL1, and (ii) induction of IFN-γ expression (FIGS. 13D and 13E).

### Example 9: Evaluation of MUC1 Inhibition in Studies in an Immune Competent MUC1 Transgenic (MUC1.Tg) Mouse Model

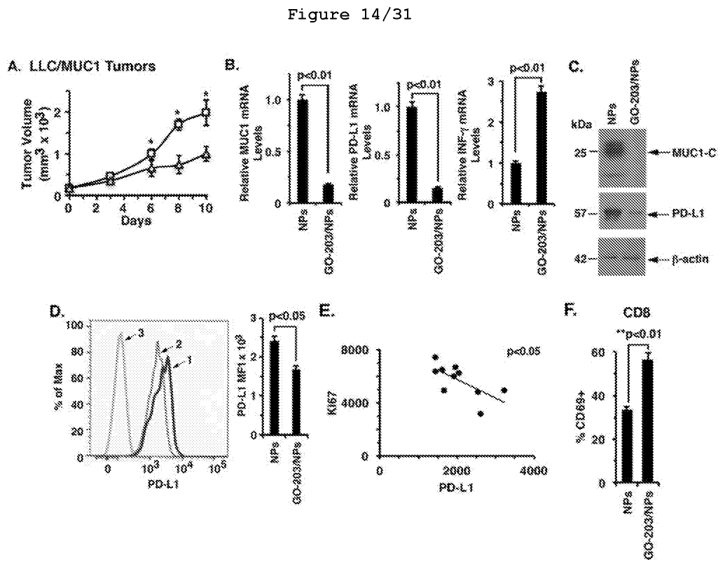

MUC1.Tg mice express the human MUC1 transgene in normal tissues in a pattern and at levels consistent with that in humans (71). MUC1.Tg mice are thus tolerant to MUC1, providing an experimental setting for the engraftment of LLC/MUC1 cells (72). MUC1.Tg mice with established LLC/MUC1 tumors were treated with GO-203/NPs to assess the effects of targeting MUC1-C on the tumor microenvironment. GO-203/NP treatment was associated with inhibition of LLC/MUC1 tumor growth as compared to that obtained with empty NPs (FIG. 14A). Analysis of the tumors on day 10 showed that targeting MUC1-C results in downregulation of MUC1 and PD-L1 mRNA levels with increases in IFN-γ expression (FIG. 14B). In addition, targeting MUC1-C resulted in the suppression of PD-L1 protein (FIG. 14C). In concert with our in vitro studies, analysis of LLC/MUC1 tumor cells by flow cytometry further demonstrated that targeting MUC1-C decreases PD-L1 expression (FIG. 14D, left and right). We also found that the expression levels of PD-L1 on tumor cells and Ki67 on T-cells were inversely correlated, suggesting that targeting MUC1-C decreases PD-L1 expression on tumors concomitant with a higher proliferative capacity of T-cells surrounding the tumor (FIG. 14E). Consistent with these results, ex-vivo analysis of TILs from the GO-203/NP-treated mice revealed that the CD69 activation marker is upregulated on CD8+ T-cells (FIG. 14F), supporting the notion that targeting MUC1-targeting MUC1-C decreases PD-L1 expression (FIG. 14D, left and right). We also found that the expression levels of PD-L1 on tumor cells and Ki67 on T-cells were inversely correlated, suggesting that targeting MUC1-C decreases PD-L1 expression on tumors concomitant with a higher proliferative capacity of T-cells surrounding the tumor (FIG. 14E). Consistent with these results, ex-vivo analysis of TILs from the GO-203/NP-treated mice revealed that the CD69 activation marker is upregulated on CD8+ T-cells (FIG. 14F) supporting the notion that targeting MUC1-C activates this population.

### Example 10: Characterization of CD8+ T-Cells in the Tumor Microenvironment

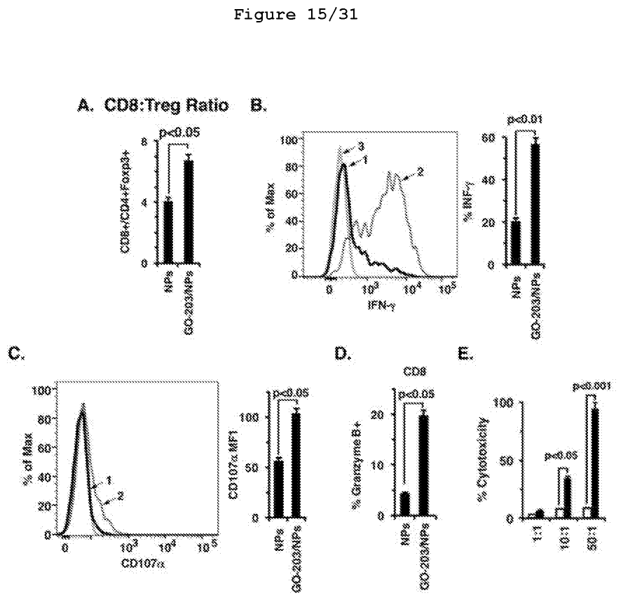

We found that GO-203/NP treatment is associated with a significant increase in the ratio of CD8+ T-cells to CD4+Foxp3+ Tregs (FIG. 15A). Moreover, and consistent with the GO-203/NP-induced upregulation of CD69, in vitro stimulation assays revealed that tumor-infiltrating CD8+ T-cells from GO-203/NP-treated mice exhibited increases in expression of IFN-γ (FIG. 15B, left and right), the degranulation marker CD107α (FIG. 15C, left and right), and granzyme B (FIG. 15D). In support of these findings indicative of enhanced function, T-cells from the GO-203/NP-, but not empty NP-, treated mice were highly effective in killing LLC/MUC1 tumor cells (FIG. 15E). These findings support the premise that targeting MUC1-C in LLC/MUC1 tumor cells with the suppression of PD-L1 is effective in restoring and potentiating tumor-infiltrating T-cell function.

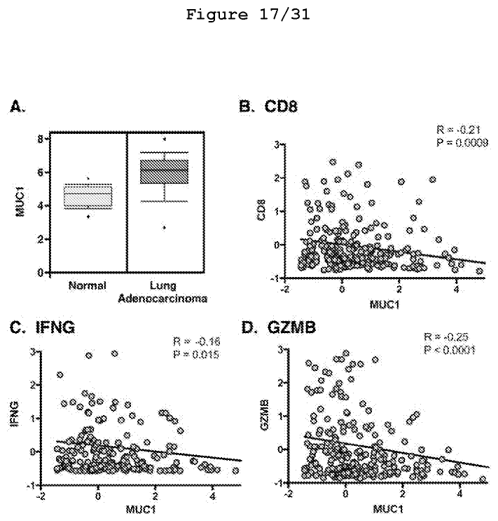

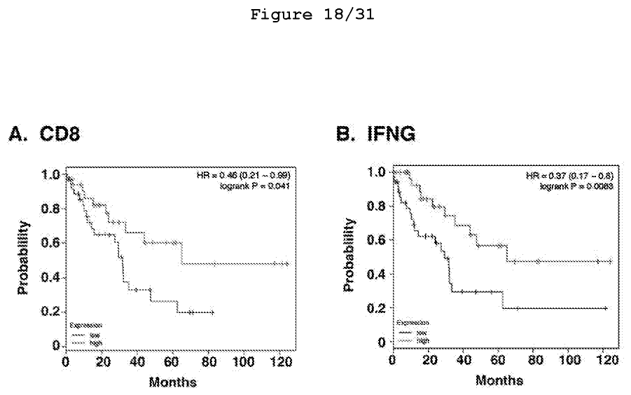

Analysis of gene expression datasets showed that MUC1 is expressed at increased levels in NSCLCs compared to that in normal tissue (FIG. 17A) and that MUC1 expression negatively correlates with CD8 (FIG. 17B; R=−0.21, p=0.0009). We also found that MUC1 expression negatively correlates with that of IFNG (FIG. 17C; R=−0.16, p=0.015) and GZMB (FIG. 17D; R=−0.25, p<0.0001), indicating that MUC1 suppresses the presence of activated CD8+ TILs in the NSCLC tumor microenvironment. Notably, lower levels of (i) CD8 (FIG. 18A; HR=0.46, p=0.041) and (ii) IFNG (FIG. 18B; HR=0.37, p=0.0083) expression in NSCLCs were associated with significant decreases in overall survival. These findings in NSCLCs thus lend further support to those obtained in LLC tumors, indicating that MUC1 plays a role in promoting immune evasion.

Aberrant overexpression of MUC1, and specifically the oncogenic MUC1-C subunit, by cancer cells has been linked with protection from killing by (i) TRAIL, (ii) Fas ligand, and (iii) T-cell perforin/granzyme B-mediated lysis (22, 23). The demonstration that MUC1-C induces PD-L1 and suppresses IFN-γ in NSCLC cells has further supported the notion that this oncoprotein integrates a program of EMT and immune evasion (73, 74). The present studies provide the first evidence that MUC1-C drives the dysregulation of PD-L1 and IFN-γ in thetumor microenvironment and that targeting MUC1-C induces cytotoxic TILs against the tumor. Notably, targeting MUC1-C with GO-203/NPs in the MUC1.Tg model had no apparent adverse effects, such as weight loss or other overt toxicity, indicating that MUC1-C is a potential target for reprogramming of the suppressive tumor microenvironment with induction of anti-tumor immunity. In this respect and regarding translational relevance, a Phase I trial of GO-203 in patients with advanced solid tumors demonstrated an acceptable safety profile. The formulation of GO-203 in NPs is now being advanced for more sustained and less frequent dosing of patients with NSCLC and other malignancies in Phase I-II studies. Based on the present findings, these GO-203/NP trials will be integrated with the administration of immune checkpoint inhibitors or other immunotherapeutic approaches.

### Example 11: General Methods: Triple Negative Breast Cancer Studies

Cell culture. Human BT-549, SUM-159 and mouse Eo771 TNBC cells were propagated in RPMI1640 medium (ATCC, Manassas, Va., USA). Human MDA-MB-468 and MDA-MB-231 TNBC cells were cultured in Dulbecco's modified Eagle's medium (DMEM) (Corning, Manassas, Va., USA). Human BT-20 TNBC cells were cultured in Eagle's Minimum Essential Medium (EMEM) (ATCC). Media were supplemented with 10% heat-inactivated fetal bovine serum, 100 units/ml penicillin, and 100 μg/ml streptomycin. Authentication of the cells was performed by short tandem repeat (STR) analysis. Cells were monitored for Mycoplasma contamination by MycoAlert® Mycoplasma Detection Kit (Lonza, Rockland, Mass., USA). BT-549 and MDA-MB-231 cells were transfected with lentiviral vectors to stably express a scrambled control shRNA (CshRNA; Sigma, St. Louis, Mo., USA) and a NF-κB p65 shRNA (Sigma). Human BT-20 and mouse Eo771 cells were stably transfected to express an empty vector or one encoding MUC1-C. Cells were treated with the IκB inhibitor BAY-11-7085 (Sigma), the BET bromodomain inhibitor JQ-1 (Delmore J E, Cell, 2011) or DMSO as the vehicle control. Cells were also treated with empty nanoparticles (NPs) or GO-203/NPs (39).

Tetracycline-inducible MUC1 and MYC silencing. MUC1shRNAs (shRNA TRCN0000122938 and shRNA #2 TRCN0000122937; MISSION shRNA; Sigma), MYCshRNA (TRCN0000039642; MISSION shRNA, Sigma) or a control scrambled CshRNA (Sigma) were cloned into the pLKO-tetpuro vector (Addgene, Cambridge, Mass., USA; Plasmid #21915). The viral vectors were co-transfected with the lentivirus packaging plasmids into 293T cells and the supernatant was collected at 48 h after transfection. BT-549 or MDA-MB-231 cells were incubated with the supernatant for 12 h in the presence of 8 μg/ml polybrene. Tet-inducible cells were selected for growth in 1-2 μg/ml puromycin and treated with doxycycline (DOX; Sigma).

Quantitative real-time, reverse transcriptase PCR (qRT-PCR). Whole cell RNA was isolated with Trizol reagent (Invitrogen, Carlsbad, Calif., USA) following the manufacturer's protocol. The High Capacity cDNA Reverse Transcription kit (Life Technologies, Carlsbad, Calif., USA) was used to synthesize cDNAs from 2 μg RNA. cDNA samples were then amplified using the Power SYBR Green PCR Master Mix (Applied Biosystems, Foster City, Calif., USA) and ABI Prism Sequence Detector Applied Biosystems). Primers used for qRT-PCR are listed in Table 1.

TABLE 1
qPCR primer sequences for RT-PCR

| | |
|---|---|
| Human GAPDH | Forward; 5′-CCATGGAGAAGGCTGGGG-3′ |
| | Reverse; 5′-CAAAGTTGTCATGGATGACC-3′ |
| Human MUC1 | Forward; 5′-TACCGATCGTAGCCCCTATG-3′ |
| | Reverse; 5′-CTCACCAGCCCAAACAGG-3′ |
| Human MUC1-C | Forward; 5′-AGACGTCAGCGTGAGTGATG-3′ |
| | Reverse; 5′-GCCAAGGCAATGAGATAGAC-3′ |
| Human PD-L1 | Forward; 5′ CCTACTGGCATTTGCTGAACGCAT-3′ |
| | Reverse; 5′-CAATAGACAATTAGTGCAGCCAGGTC-3′ |
| Mouse PD-L1 | Forward; 5′-TGCTGCATAATCAGCTACGG-3′ |
| | Reverse; 5′-GCTGGTCACATTGAGAAGCA-3′ |
| Mouse 36B4 | Forward; 5′-CTGTTGGCCAATAAGGTGCC-3′ |
| | Reverse; 5′ GTTCTGAGCTGGCACAGTGA-3′ |

Immunoblot analysis. Whole cell extracts were obtained using NP-40 buffer composed of 50 mM Tris-HCl (pH 7.4), 150 mM NaCl, 1% NP-40, protease inhibitor cocktail and DTT. Immunoblotting was performed with anti-MUC1-C (ThermoFischer Scientific, Waltham, Mass., USA), anti-PD-L1, anti-MYC, anti-phospho-p65 (Ser-536) (Cell Signaling Technology, Danvers, Mass., USA), anti-NF-κB p65 (Santa Cruz Biotechnology, Dallas, Tex.), mouse PD-L1 (Bio-Techne, Minneapolis, Minn., USA) and anti-β-actin (Sigma). Immunoreactive complexes were detected using horseradish peroxidase-conjugated secondary antibodies (GE Healthcare Life Sciences, Marlborough, Mass., USA) and an enhanced chemiluminescence (ECL) detection reagents (Perkin Elmer Health Sciences, Waltham, Mass., USA). Promoter-reporter assays. Cells were transfected with 1.5 μg of PD-L1 promoter-luciferase reporter (pPD-L1-Luc) or control vector (Active Motif, Carlsbad, Calif., USA) in the presence of Superfect (Qiagen, Germantown, Md., USA). After 48 h, the cells were lysed in passive lysis buffer. Lysates were analyzed using the Lightswitch Luciferase Assay Kit (Active Motif).

Promoter-reporter assays. Cells were transfected with 1.5 μg of PD-L1 promoter-luciferase reporter (pPD-L1-Luc) or control vector (Active Motif, Carlsbad, Calif., USA) in the presence of Superfect (Qiagen, Germantown, Md., USA). After 48 h, the cells were lysed in passive lysis buffer. Lysates were analyzed using the Lightswitch Luciferase Assay Kit (Active Motif).

Chromatin immunoprecipitation (ChIP) assays. Soluble chromatin was prepared from 3×106 cells and precipitated with anti-MYC, anti-NF-κB p65 (Santa Cruz Biotechnology), anti-MUC1-C or a control nonimmune IgG. Power SYBR Green PCR Master Mix (Applied Biosystems) and ABI Prism Sequence Detector (Applied Biosystems) were used for amplification of ChIP qPCRs. Primers used for qPCR of the PD-L1 promoter and GAPDH control region are listed in Table 2. Relative fold enrichment was calculated as described (44).

TABLE 2
ChIP qPCR Priner Pairs

| | |
|---|---|
| GAPDH Promoter | Forward; 5′ TACTAGCGGTTTTACGGGCG-3′ |
| | Reverse; 5′ TCGAACAGGAGGAGCAGAGAGCGA-3′ |
| PD-L1 Promoter | Forward; 5′-CATATGGGTCTGCTGCTGAC-3′ |
| | Reverse; 5′-CAACAAGCCAACATCTGAAC-3′ |

Mouse model studies Eo771/MUC1-C cells (0.5×10⁶ cells) were subcutaneously injected into the flanks of six-week old human MUC1.Tg mice. After reaching a tumor size of ˜150 mm3, mice were pair-matched into two groups and treated with empty NPs or 15 mg/kg GO-203/NPs once a week for 2 weeks. At the end of the treatment, mice were sacrificed for harvesting of the tumors. In an additional experiment, mice bearing Eo771/MUC1-C tumors were treated with vehicle control (PBS) or 10 mg/kg anti-PD-L1 (BioXCell, West Lebanon, N.H., USA) on days 1 and 5 as described (75). Animal care was performed in accordance with Dana-Farber Cancer Institute guidelines for animal experiments.

FACS analysis. Eo771/MUC1-C tumors were harvested, cut into small pieces and incubated in dissociation medium containing 100 units/ml Collagenase IV (ThermoFisher Scientific, Grand Island, N.Y., USA) and 50 μg/ml DNase I (Roche, Indianapolis, Ind., USA) for 30 min at 37° C. Tumor cell suspensions were passed through 70 μm strainers (ThermoFisher Scientific). After lysis of red blood cells with ACK buffer (ThermoFisher Scientific), tumor cells were counted, and an aliquot of each sample was analyzed by FACS staining for CD69 and granzyme B (BioLegend, San Diego, Calif., USA) expression on CD8+ T-cells (BD LSR II Flow Cytometer, BD Pharmingen, San Diego, Calif., USA). Spleen cells were used for adjusting compensation during the analysis. After Ficoll separation, 3×10⁶ cells were incubated with Leucocyte Activation Cocktail (BD Pharmingen) and Alexa 488 labeled anti-mouse CD107α antibody (BioLegend) for 6 h at 37° C. Cells were processed for FACS analysis of IFN-γ (ThermoFisher Scientific), granzyme B and CD107α.

CTL assays. The day before mice sacrifice, Eo771/MUC1-C cells (6×10³ per well) were plated in 96-well plates and incubated overnight. Lymph nodes were digested with ACK lysis buffer (GIBCO, Waltham, Mass., USA) and rinsed with PBS. Cells (effector cells) were incubated with Eo771/MUC1-C cells (target cells) in 96 well-plates for 6 h. The percentage cytotoxicity was assayed measuring LDH release following the manufacturer's recommendations (CytoTox 96® Non-Radioactive Cytotoxicity Assay; Promega, Madison, Wis., USA) and calculated using the formula: (Experimental-Effector spontaneous−Target spontaneous)/(Target maximum−Target spontaneous)×100.

Bioinformatic analyses. Datasets of TNBC patients were downloaded from the Gene Expression Omnibus (GEO) under the accession number GSE25066 (76). Raw signal intensities were RMA normalized across patients (77). Multiple probe sets corresponding to the same gene were averaged. Expression values of MUC1, CD8, CD69 and GZMB in TNBC samples were assessed for correlations using the Spearman coefficient. The prognostic value of CD8, CD69 and GZMB expression in TNBC datasets was determined as described (66). Expression values were averaged and TNBC patients were segregated by median expression. The Kaplan-Meier survival probability plot with the hazard ratio (95% confidence interval) and log-rank p-value were calculated and plotted in R.

Statistical analysis. Analyses were performed using GraphPad Prism version 7.0 (GraphPad Software Inc, San Diego, Calif., USA) and p values <0.05 were considered statistically significant differences.

### Example 12: MUC1 Drives PD-L1 Expression in TNBC Cells

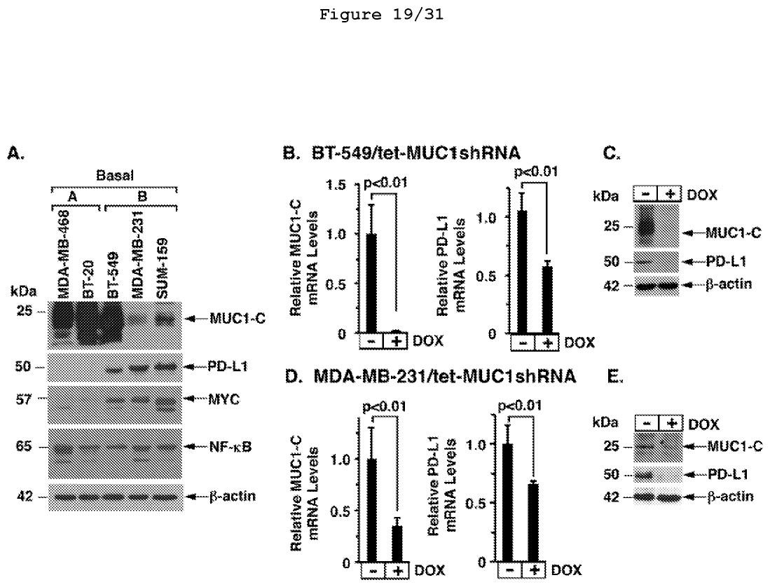

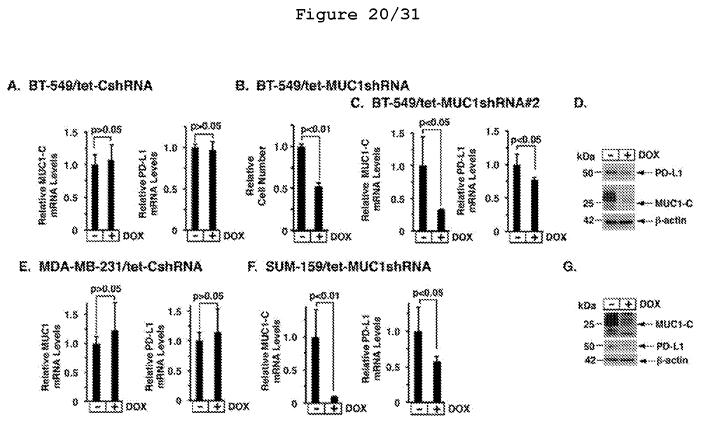

MUC1-C induces the EMT state, CSC characteristics and epigenetic reprogramming in basal B TNBC cells (44, 94, 96-98, 99). To investigate the potential relationships between MUC1-C and PD-L1, we first performed immunoblot analysis of TNBC cell lines and found readily detectable PD-L1 levels in the mesenchymal basal B BT-549, MDA-MB-231 and SUM159 cells, as compared to that in basal A MDA-MB-468 and BT-20 cells (FIG. 19A). The results further showed that, in contrast to NF-κB p65, MYC is upregulated in basal B, but not basal A, TNBC cells (FIG. 19A). We therefore established BT-549, MDA-MB-231 and SUM159 cells with stable expression a tetracycline-inducible control shRNA (tet-CshRNA) or MUC1 shRNA (tet-MUC1shRNA) to determine whether MUC1-C contributes to the regulation of PD-L1 expression. As a control, doxycycline (DOX) treatment of BT-549/tet-CshRNA cells had no effect on MUC1 or PD-L1 expression (FIG. 20A). By contrast, treatment of BT-549/tet-MUC1 shRNA cells with DOX was associated with slowing of proliferation (FIG. 20B) and downregulation of MUC1-C and PD-L1 mRNA (FIG. 19B) and protein (FIG. 19C). In addition, DOX treatment of BT-549/tet-MUC1shRNA #2 cells expressing a different MUC1 shRNA resulted in downregulation of PD-L1 expression (FIGS. 20C and 20D). Similar results were obtained with DOX-treated (i) MDA-MB-231/tet-CshRNA (FIG. 20E) and MDA-MB-231/tet-MUC1shRNA (FIGS. 19D and 19E) cells, and (ii) SUM-159/tet-MUC1shRNA (FIGS. 20F and 20G) cells, further supporting the premise that MUC1-C promotes the induction of PD-L1 expression.

### Example 13: Targeting the MUC1-C Cytoplasmic Domain Downregulates PD-L1 Expression

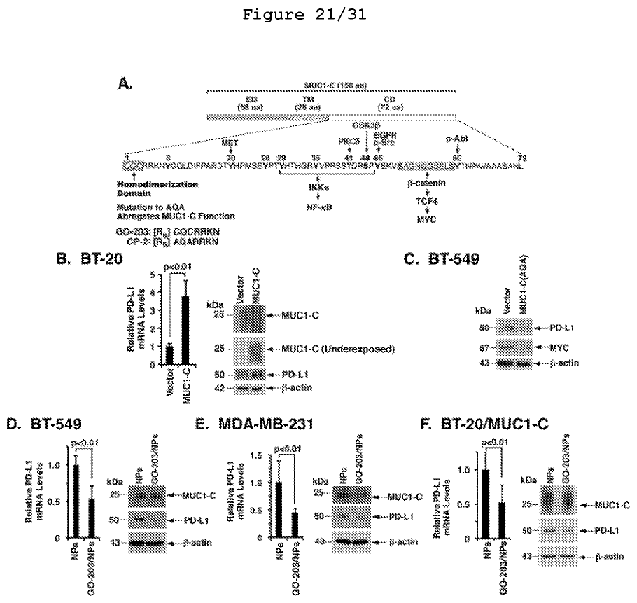

The MUC1-C subunit consists of a 58-amino acid (aa) ectodomain, a 28-aa transmembrane domain, and a 72-aa intrinsically disordered cytoplasmic domain (CD) (FIG. 21A). Enforced expression of MUC1-C has been linked to the induction of EMT (44). In concert with these and the above findings, overexpression of MUC1-C in BT-20 cells also resulted in upregulation of PD-L1 expression (FIG. 21B, left and right; an underexposed blot is shown to document MUC1-C upregulation), demonstrating that MUC1-C, and not the shed MUC1-N subunit, is sufficient for this response. Of note, the MUC1-C cytoplasmic domain includes a CQC motif (FIG. 21A), which is essential for the formation of MUC1-C homodimers and their import into the nucleus (23,35). In this regard, mutation of the CQC motif to AQA abrogates MUC1-C function (38) and, in the present studies, expression of the MUC1-C (AQA) mutant in BT-549 cells (98) resulted in downregulation of PD-L1 expression (FIG. 21C). The findings that the CQC motif is of importance to MUC1-C signaling provided the basis for developing the cell-penetrating GO-203 peptide to target this site (FIG. 2A) (36,37). In addition, GO-203 has been encapsulated in polymeric nanoparticles (GO-203/NPs) for sustained delivery in vitro and in animal models (39). Treatment of BT-549 (FIG. 21D, left and right) and MDA-MB-231 (FIG. 21E, left and right) cells with GO-203/NPs, but not empty NPs, was associated with downregulation of PD-L1 expression. Moreover, studies in BT-20 cells with MUC1-C overexpression (BT-20/MUC1-C) further demonstrated that targeting MUC1-C with GO-203 results in suppression of PD-L1 mRNA and protein (FIG. 21F, left and right). These findings thus demonstrated that MUC1-C is sufficient for the induction of PD-L1 expression and that this pathway is inhibited by targeting the MUC1-C CQC motif.

### Example 14: MUC1-C Drives PD-L1 by a MYC-Dependent Mechanism

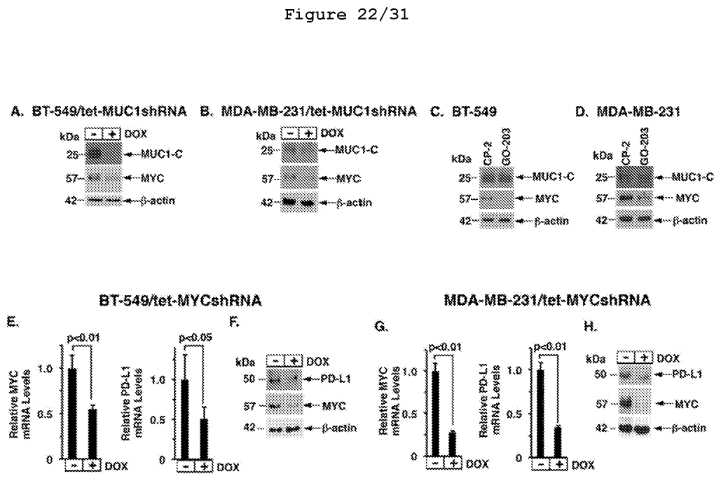

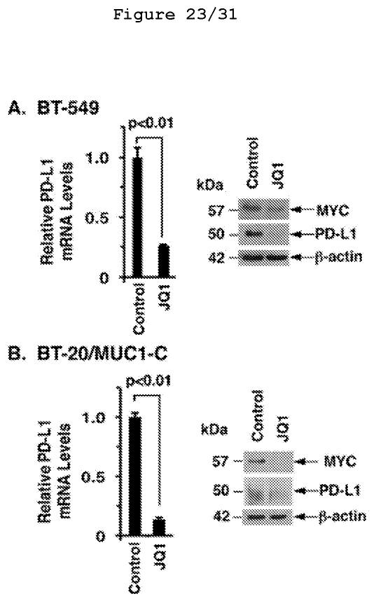

MUC1-C is associated with the upregulation of MYC (49,77) and drives MYC mediated epigenetic reprogramming (98); however, there is no known relationship between MUC1-C→MYC signaling and PD-L1. In searching for evidence, we found that DOX treatment of BT-549/tet-MUC1shRNA (FIG. 22A) and MDA-MB-231/tet-MUC1shRNA (FIG. 22B) cells results in the downregulation of MYC expression. Treatment of BT-549 (FIG. 22C) and MDA-MB-231 (FIG. 22D) cells with GO-203, but not the control CP-2, was also associated with the suppression of MYC, supporting the premise that MUC1-C induces MYC expression in TNBC cells. To determine if MYC drives PD-L1 in TNBC cells, we established BT-549 and MDA-MB-231 cells with stable expression of a tet-MYCshRNA. DOX treatment of BT-549/tet-MYCshRNA was associated with suppression of MYC and PD-L1 mRNA (FIG. 22E, left and right) and protein (FIG. 22F). Similar results were obtained in DOX-treated MDA-MB-231/tet-MYCshRNA cells (FIG. 22G, left and right; and FIG. 22H). Treatment of BT-549 with JQ1, a BET bromodomain inhibitor, was also associated with downregulation of PD-L1 expression in BT-549 (FIG. 23A) and BT-20/MUC1-C (FIG. 23B) cells, providing further support for a MUC1-C→MYC→PD-L1 pathway in basal B TNBC cells.

### Example 15: MUC1-C Induces PD-L1 Expression by the NF-κB p65 Pathway

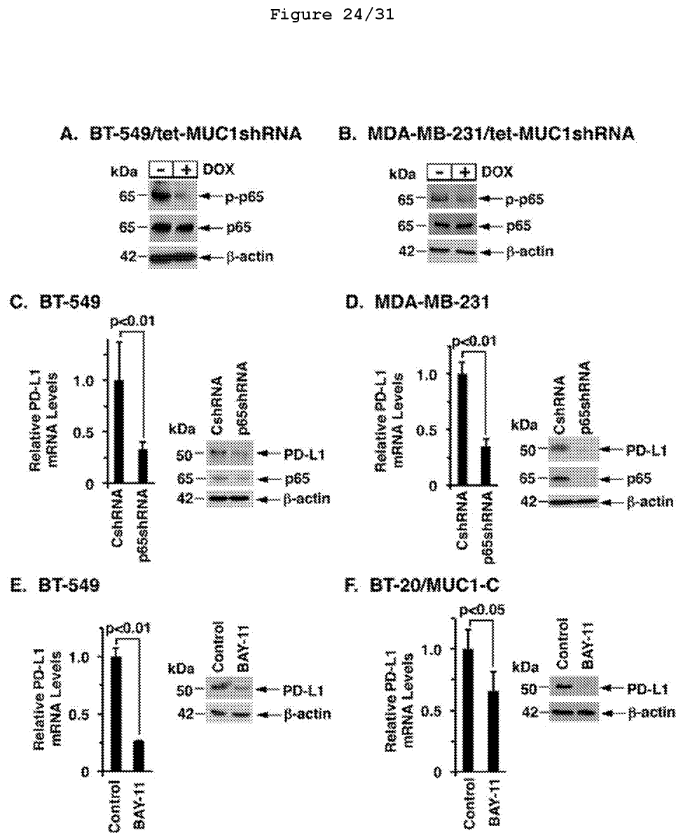

MUC1-C activates the proinflammatory TAK1→IKK→NF-κB p65 pathway in cancer cells (FIG. 21A) (32, 33, 100). MUC1-C also binds directly to NF-κB p65 and thereby drives its downstream target genes, including (i) MUC1 itself in an autoinductive loop (33), and (ii) ZEB1 with activation of the EMT program in basal B TNBC cells (44). To investigate whether MUC1-C activates PD-L1 by an NF-κB p65-mediated pathway, we first showed that downregulation of MUC1-C in DOX-treated BT-549/tet-MUC1shRNA (FIG. 24A) and MDA-MB-231/tet-MUC1 shRNA (FIG. 24B) cells results in the suppression of phosphop65, but not p65, levels. To extend this analysis, we established BT-549 and MDA-MB-231 cells expressing a p65shRNA. Targeting NF-κB p65 in BT-549/p65shRNA (FIG. 24C, left and right) and MDA-MB-231/p65shRNA (FIG. 24D, left and right) cells was associated with decreases in PD-L1 mRNA and protein, indicating that MUC1-C drives PD-L1 expression by the NF-κB p65 pathway. In support of this contention, treatment of BT-549 (FIG. 24E, left and right) and BT-20/MUC1-C (FIG. 24F, left and right) cells with BAY-11-7085, an irreversible inhibitor of IκB phosphorylation, resulted in suppression of PD-L1 expression.

### Example 16: MUC1-C Enhances MYC and NF-κB p65 Occupancy on the PD-L1 Promoter

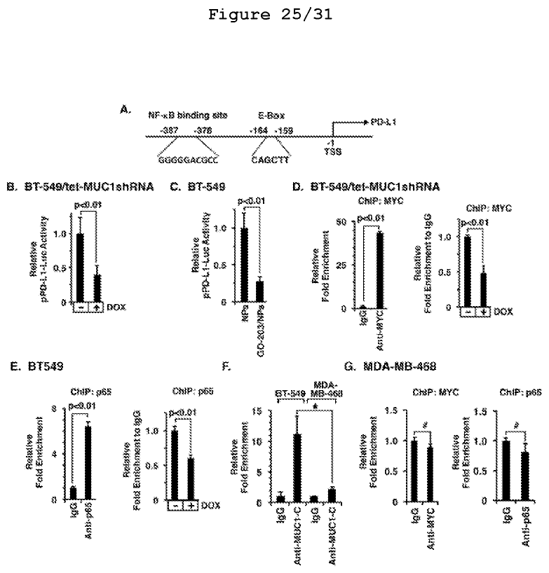

The PD-L1 promoter contains (i) an E-box sequence (CAGCTT) for MYC binding at positions −164 to −159, and (ii) an NF-κB p65 binding site (GGGGGACGCC) at positions −387 to −378 upstream to the transcription start site (FIG. 25A) (49). To determine whether MUC1-C activates the PD-L1 promoter, we transfected DOX-treated BT-549/tet-MUC1shRNA cells with a PD-L1 promoter-luciferase reporter (pPD-L1-Luc). The results demonstrated that silencing MUC1-C suppresses pPD-L1-Luc activity (FIG. 25B). Targeting MUC1-C in BT-549 cells with GO-203/NP treatment also decreased activity of the pPD-L1-Luc reporter (FIG. 25C), indicating that MUC1-C activates the PD-L1 promoter. To our knowledge, it is not known if MYC or NF-κB p65 occupies the PD-L1 promoter in TNBC cells. Accordingly, we performed ChIP studies of chromatin from BT-549/tet-MUC1shRNA cells which demonstrated that MYC is detectable on the PD-L1 promoter (FIG. 25D, left) and that silencing MUC1-C decreases MYC occupancy (FIG. 25D, right). In a similar way, we found that NF-κB p65 also occupies the PD-L1 promoter by a MUC1-C dependent mechanism (FIG. 25E, left and right). Notably, MUC1-C occupancy on the PD-L1 promoter was substantially greater in basal B BT-549 cells as compared to that found in basal A MDA-MB-468 cells, which have low to undetectable levels of PD-L1 expression (FIG. 25F). In addition, there was no significant detection of MYC or NF-κB p65 occupancy on the PD-L1 promoter in MDA-MB-468 cells (FIG. 25G, left and right), providing mechanistic evidence for the findings that MUC1-C drives PD-L1 in basal B, and not basal A, TNBC cells.

### Example 17: MUC1-C Drives PD-L1

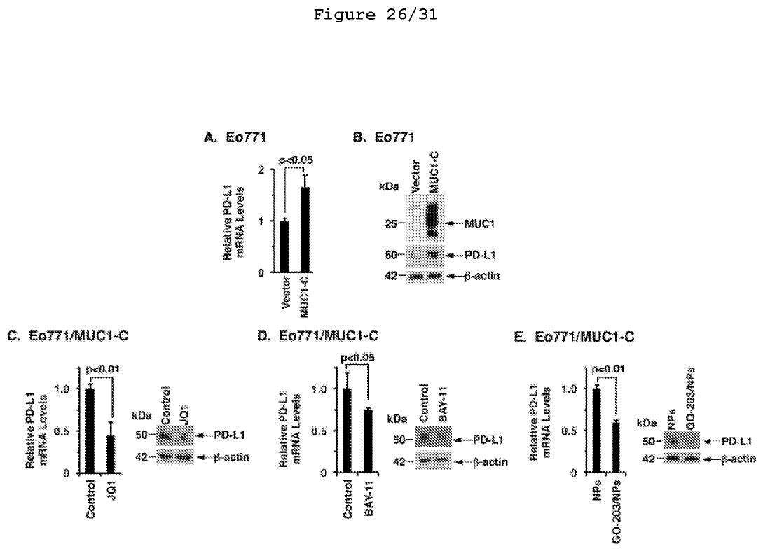

To extend this line of investigation, we studied mouse Eo771 TNBC cells stably expressing human MUC1-C (Eo771/MUC1-C). Notably, Eo771/MUC1-C cells exhibited increased levels of PD-L1 mRNA (FIG. 26A) and protein (FIG. 26B) relative to that in control cells expressing an empty vector (Eo771/vector). In concert with the above studies in human TNBC cells, we also found that the MUC1-C→PD-L1 response is inhibited by treatment with JQ1 (Fig. S26C, left and right) and BAY-11 (FIG. 26D, left and right). In addition, treatment of the Eo771/MUC1-C cells with GO-203/NPs was associated with downregulation of PD-L1 mRNA and protein (FIG. 26E, left and right), confirming that MUC1-C drives PD-L1 expression in mouse Eo771 cells by MYC- and NF-κB p65-mediated mechanisms.

### Example 18: Targeting MUC1-C in Suppresses PD-L1 Expression and Activates the Tumor Immune Microenvironment

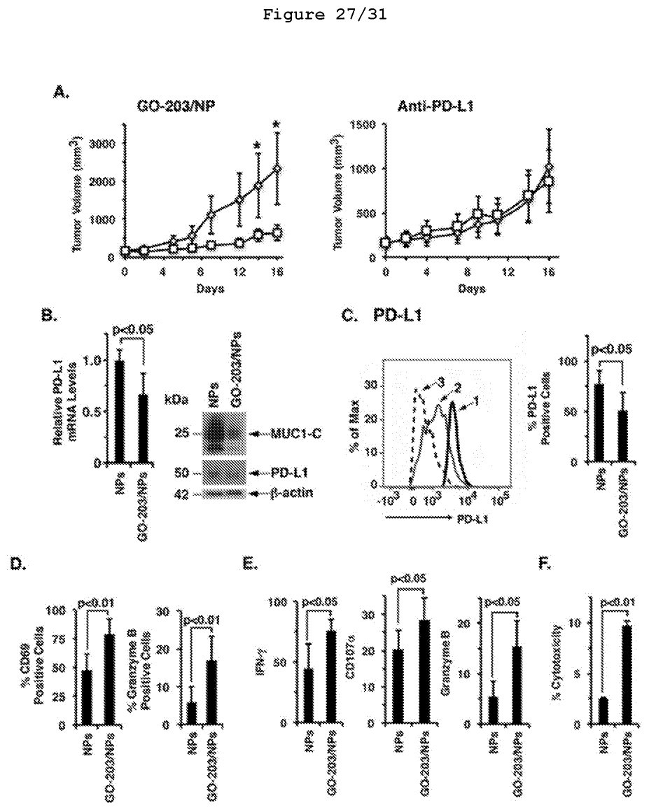

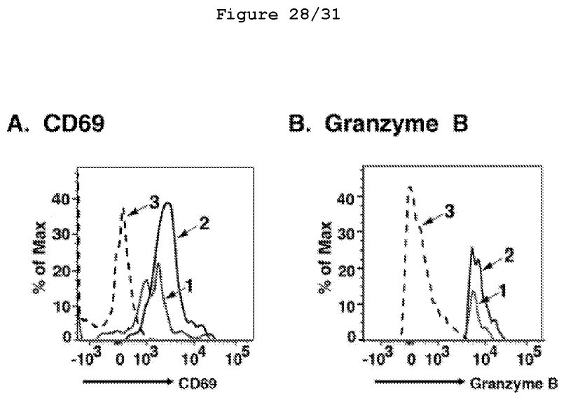

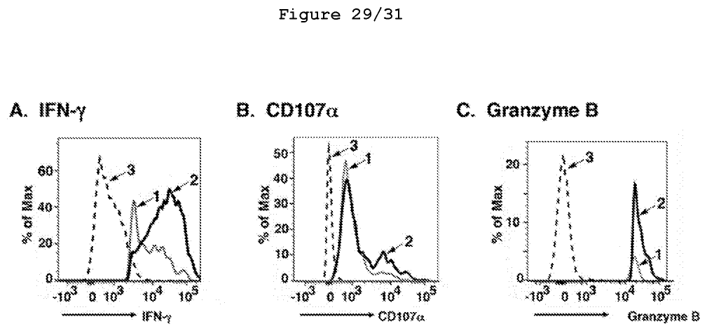

We next performed studies in the human MUC1 transgenic (MUC1.Tg) mouse model. The immune competent MUC1.Tg mice express the MUC1 transgene in normal tissues in a pattern and at levels consistent with that in humans (71). In addition, MUC1.Tg mice are tolerant to MUC1, thereby providing an experimental setting for engraftment of Eo771/MUC1-C cells. MUC1.Tg mice with established Eo771/MUC1-C tumors were treated with GO-203/NPs to assess the effects of targeting MUC1-C on the tumor microenvironment. GO-203/NP, but not anti-PD-L1, treatment was associated with inhibition of Eo771/MUC1-C tumor growth as compared to that obtained with respective controls (FIG. 27A, left and right). Analysis of the GO-203/NP-treated tumors on day 16 showed that targeting MUC1-C results in the downregulation of PD-L1 mRNA and protein (FIG. 27B, left and right). In addition, GO-203/NP treatment decreased PD-L1 expression on the Eo771/MUC1-C cell surface (FIG. 27C, left and right). Analysis of the TIL population also revealed that expression of the CD69 activation marker and granzyme B is upregulated in CD8+ T-cells after GO-203/NP treatment (FIG. 27D, left and right; FIGS. 28A and 28B). Moreover, and consistent with these results, in vitro stimulation assays demonstrated that tumor infiltrating CD8+ T-cells from GO-203/NP-treated mice exhibit increases in expression of IFN-γ (FIG. 27E, left; FIG. 29A), the degranulation marker CD107α (FIG. 27E, middle; FIG. 29B) and granzyme B (FIG. 27E, right; FIG. 29C). In support of these findings indicative of enhanced function, TILs from the GO-203/NP-treated mice were more effective in killing Eo771/MUC1-C tumor cells (FIG. 27F).

### Example 19: Correlation of MUC1 with T-Cell Activation in TNBC

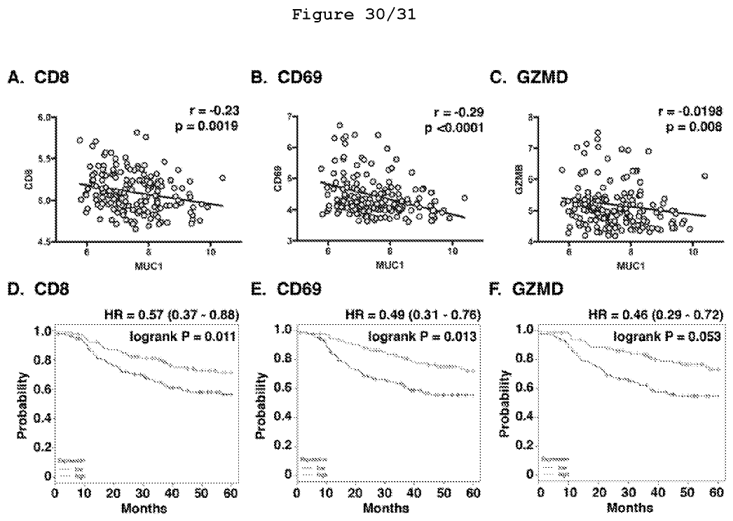

To further understand the relationship between MUC1-C and T-cell activation in TNBCs, we performed bioinformatics analyses on the microarray dataset from the Gene Expression Omnibus (GSE25066). The results demonstrated that MUC1 expression correlates inversely with that obtained for CD8 (FIG. 30A), CD69 (FIG. 30B) and GZMB (FIG. 30C). Additionally, we found that higher levels of CD8 (FIG. 30D), CD69 (FIG. 30E) and GZMB (FIG. 30F) expression are associated with significant increases in disease-free survival of TNBC patients.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

### REFERENCES

1. Hanahan, D., and Weinberg, R. A. 2011. Hallmarks of cancer: the next generation. Cell 144:646-674.
2. Balkwill, F., Charles, K. A., and Mantovani, A. 2005. Smoldering and polarized inflammation in the initiation and promotion of malignant disease. Cancer Cell 7:211-217.
3. Carrizosa, D. R., and Gold, K. A. 2015. New strategies in immunotherapy for non-small cell lung cancer. Transl Lung Cancer Res 4:553-559.
4. Reck, M., and Paz-Ares, L. 2015. Immunologic checkpoint blockade in lung cancer. Semin Oncol 42:402-417.
5. Mayor, M., Yang, N., Sterman, D., Jones, D. R., and Adusumilli, P. S. 2015. Immunotherapy for non-small cell lung cancer: current concepts and clinical trials. Eur J Cardiothorac Surg 49:1324-1333.
6. Akbay, E. A., Koyama, S., Carretero, J., Altabef, A., Tchaicha, J. H., Christensen, C. L., Mikse, O. R., Chemiack, A. D., Beauchamp, E. M., Pugh, T. J., et al. 2013. Activation of the PD-1 pathway contributes to immune escape in EGFR-driven lung tumors. Cancer Discov 3:1355-1363.
7. Koyama S, Akbay E A, Li Y Y, Aref A R, Skoulidis F, Herter-Sprie G S, Buczkowski K A, Liu Y, Awad M M, Denning W L, et al. 2016. STK11/LKB1 deficiency promotes neutrophil recruitment and proinflammatory cytokine production to suppress T cell activity in the lung tumor microenvironment. Cancer Res 76:999-1008.
8. Gong, W., Song, Q., Lu, X., Gong, W., Zhao, J., Min, P., and Yi, X. 2011. Paclitaxel induced B7-H1 expression in cancer cells via the MAPK pathway. J Chemother 23:295-299.
9. Qin, X., Liu, C., Zhou, Y., and Wang, G. 2010. Cisplatin induces programmed death-1-ligand 1 (PD-L1) over-expression in hepatoma H22 cells via Erk/MAPK signaling pathway. Cell Mol Biol (Noisy-le-grand) 56 Suppl:OL1366-1372.
10. Jiang, X., Zhou, J., Giobbie-Hurder, A., Wargo, J., and Hodi, F. S.
2013. The activation of MAPK in melanoma cells resistant to BRAF inhibition promotes PD-L1 expression that is reversible by MEK and PI3K inhibition. Clin Cancer Res 19:598-609.
11. Song, M., Chen, D., Lu, B., Wang, C., Zhang, J., Huang, L., Wang, X., Timmons, C. L., Hu, J., Liu, B., et al. 2013. PTEN loss increases PD-L1 protein expression and affects the correlation between PD-L1 expression and clinical parameters in colorectal cancer. PLoS One 8:e65821.
12. Noman, M. Z., and Chouaib, S. 2014. Targeting hypoxia at the forefront of anticancer immune responses. Oncoimmunology 3:e954463.
13. Marzec, M., Zhang, Q., Goradia, A., Raghunath, P. N., Liu, X., Paessler, M., Wang, H. Y., Wysocka, M., Cheng, M., Ruggeri, B. A., et al. 2008. Oncogenic kinase NPM/ALK induces through STAT3 expression of immunosuppressive protein CD274 (PD-L1, B7-H1). Proc Natl Acad Sci USA 105:20852-20857.
14. Casey, S. C., Tong, L., Li, Y., Do, R., Walz, S., Fitzgerald, K. N., Gouw, A. M., Baylot, V., Gutgemann, I., Eilers, M., et al. 2016. MYC regulates the antitumor immune response through CD47 and PD-L1. Science 352:227-231.
15. Chen, L., Gibbons, D. L., Goswami, S., Cortez, M. A., Ahn, Y. H., Byers, L. A., Zhang, X., Yi, X., Dwyer, D., Lin, W., et al. 2014. Metastasis is regulated via microRNA-200/ZEB1 axis control of tumour cell PD-L1 expression and intratumoral immunosuppression. Nat Commun 5:5241.
16. Situ, D., Wang, J., Ma, Y., Zhu, Z., Hu, Y., Long, H., and Rong, T. 2010. Expression and prognostic relevance of MUC1 in stage IB non-small cell lung cancer. Med. Oncol. 28:596-604.
17. Jarrard, J. A., Linnoila, R. I., Lee, H., Steinberg, S. M., Witschi, H. and Szabo, E. 1998. MUC1 is a novel marker for the type II pneumocyte lineage during lung carcinogenesis. Cancer Res 58:5582-5589.
18. Awaya, H., Takeshima, Y., Yamasaki, M., and Inai, K. 2004. Expression of MUC1, MUC2, MUC5AC, and MUC6 in atypical adenomatous hyperplasia, bronchioloalveolar carcinoma, adenocarcinoma with mixed subtypes, and mucinous bronchioloalveolar carcinoma of the lung. Am. J. Clin. Pathol. 121:644-653.
19. Khodarev, N., Pitroda, S., Beckett, M., MacDermed, D., Huang, L., Kufe, D., and Weichselbaum, R. 2009. MUC1-induced transcriptional programs associated with tumorigenesis predict outcome in breast and lung cancer. Cancer Res. 69:2833-2837.
20. MacDermed D M, Khodarev N N, Pitroda S P, Edwards D C, Pelizzari C A, Huang L, Kufe D W, and Weichselbaum, R. 2010. MUC1-associated proliferation signature predicts outcomes in lung adenocarcinoma patients. BMC Medical Genomics 3:16.
21. Xu, F., Liu, F., Zhao, H., An, G., and Feng, G. 2015. Prognostic significance of mucin antigen MUC1 in various human epithelial cancers: a meta-analysis. Medicine (Baltimore) 94:e2286.
22. Kufe, D. 2009. Mucins in cancer: function, prognosis and therapy. Nature Reviews Cancer 9:874-885.
23. Kufe, D. 2013. MUC1-C oncoprotein as a target in breast cancer: activation of signaling pathways and therapeutic approaches. Oncogene 32:1073-1081.
24. Ramasamy, S., Duraisamy, S., Barbashov, S., Kawano, T., Kharbanda, S., and Kufe, D. 2007. The MUC1 and galectin-3 oncoproteins function in a microRNA-dependent regulatory loop. Mol. Cell 27:992-1004.
25. Raina, D., Agarwal, P., Lee, J., Bharti, A., McKnight, C., Sharma, P., Kharbanda, S., and Kufe, D. 2015. Characterization of the MUC1-C cytoplasmic domain as a cancer target. PLoS One 10:e0135156.
26. Raina, D., Kharbanda, S., and Kufe, D. 2004. The MUC1 oncoprotein activates the anti-apoptotic PI3K/Akt and Bcl-xL pathways in rat 3Y1 fibroblasts. J Biol Chem 279:20607-20612.
27. Kharbanda, A., Rajabi, H., Jin, C., Tchaicha, J., Kikuchi, E., Wong, K., and Kufe, D. 2014. Targeting the oncogenic MUC1-C protein inhibits mutant EGFR-mediated signaling and survival in non-small cell lung cancer cells. Clin Cancer Res 20:5423-5434.
28. Kharbanda, A., Rajabi, H., Jin, C., Alam, M., Wong, K., and Kufe, D. 2014. MUC1-C confers EMT and KRAS independence in mutant KRAS lung cancer cells. Oncotarget 5:8893-8905.
29. Rajabi, H., Ahmad, R., Jin, C., Kosugi, M., Leng, M., Joshi, M., and Kufe, D. 2012. MUC1-C oncoprotein induces TCF7L2 transcription factor activation and promotes cyclin D1 expression in human breast cancer cells. J Biol Chem 287:10703-10713.
30. Khodarev, N., Ahmad, R., Rajabi, H., Pitroda, S., Kufe, T., McClary, C., Joshi, M. D., MacDermed, D., Weichselbaum, R., and Kufe, D. 2010. Cooperativity of the MUC1 oncoprotein and STAT1 pathway in poor prognosis human breast cancer. Oncogene 29:920-929.
31. Ahmad, R., Rajabi, H., Kosugi, M., Joshi, M., Alam, M., Vasir, B., Kawano, T., Kharbanda, S., and Kufe, D. 2011. MUC1-C oncoprotein promotes STAT3 activation in an auto-inductive regulatory loop. Science Signaling 4:ra9.
32. Ahmad, R., Raina, D., Trivedi, V., Ren, J., Rajabi, H., Kharbanda, S., and Kufe, D. 2007. MUC1 oncoprotein activates the IκB kinase β complex and constitutive NF-κB signaling. Nat Cell Biol 9:1419-1427.
33. Ahmad, R., Raina, D., Joshi, M. D., Kawano, T., Kharbanda, S., and Kufe, D. 2009. MUC1-C oncoprotein functions as a direct activator of the NF-κB p65 transcription factor. Cancer Res 69:7013-7021.
34. Takahashi, H., Jin, C., Rajabi, H., Pitroda, S., Alam, M., Ahmad, R., Raina, D., Hasegawa, M., Suzuki, Y., Tagde, A., et al. 2015. MUC1-C activates the TAK1 inflammatory pathway in colon cancer. Oncogene 34:5187-5197.
35. Leng, Y., Cao, C., Ren, J., Huang, L., Chen, D., Ito, M., and Kufe, D. 2007. Nuclear import of the MUC1-C oncoprotein is mediated by nucleoporin Nup62. J Biol Chem 282:19321-19330.
36. Raina, D., Ahmad R, Rajabi H, Panchamoorthy G, Kharbanda S, and Kufe, D. 2012. Targeting cysteine-mediated dimerization of the MUC1-C oncoprotein in human cancer cells. Int J Oncol 40:1643-1649.
37. Raina, D., Kosugi, M., Ahmad, R., Panchamoorthy, G., Rajabi, H., Alam, M., Shimamura, T., Shapiro, G., Supko, J., Kharbanda, S., et al. 2011. Dependence on the MUC1-C oncoprotein in non-small cell lung cancer cells. Mol Cancer Ther 10:806-816.
38. Kufe, D. 2009. Functional targeting of the MUC1 oncogene in human cancers. Cancer Biol Ther 8:1201-1207.
39. Hasegawa, M., Sinha, R. K., Kumar, M., Alam, M., Yin, L., Raina, D., Kharbanda, A., Panchamoorthy, G., Gupta, D., Singh, H., et al. 2015. Intracellular targeting of the oncogenic MUC1-C protein with a novel GO-203 nanoparticle formulation. Clin Cancer Res 21:2338-2347.
40. Cherfils-Vicini, J., Platonova, S., Gillard, M., Laurans, L., Validire, P., Caliandro, R., Magdeleinat, P., Mami-Chouaib, F., Dieu-Nosjean, M. C., Fridman, W. H., et al. 2010. Triggering of TLR7 and TLR8 expressed by human lung cancer cells induces cell survival and chemoresistance. J Clin Invest 120:1285-1297.
41. Chatterjee, S., Crozet, L., Damotte, D., Iribarren, K., Schramm, C., Alifano, M., Lupo, A., Cherfils-Vicini, J., Goc, J., Katsahian, S., et al. 2014. TLR7 promotes tumor progression, chemotherapy resistance, and poor clinical outcomes in non-small cell lung cancer. Cancer Res 74:5008-5018.
42. Dajon, M., Iribarren, K., and Cremer, I. 2015. Dual roles of TLR7 in the lung cancer microenvironment. Oncoimmunology 4:e991615.
43. Lee, J., Hayashi, M., Lo, J. F., Fearns, C., Chu, W. M., Luo, Y., Xiang, R., and Chuang, T. H. 2009. Nuclear factor kappaB (NF-kappaB) activation primes cells to a pro-inflammatory polarized response to a Toll-like receptor 7 (TLR7) agonist. Biochem J 421:301-310.
44. Rajabi, H., Alam M, Takahashi H, Kharbanda A, Guha M, Ahmad R, and D., K. 2014. MUC1-C oncoprotein activates the ZEB1/miR-200c regulatory loop and epithelial-mesenchymal transition. Oncogene 33:1680-1689.
45. Takeshita, F., Suzuki, K., Sasaki, S., Ishii, N., Klinman, D. M., and Ishii, K. J. 2004. Transcriptional regulation of the human TLR9 gene. J Immunol 173:2552-2561.
46. Deshmane, S. L., Kremlev, S., Amini, S., and Sawaya, B. E. 2009. Monocyte chemoattractant protein-1 (MCP-1): an overview. J Interferon Cytokine Res 29:313-326.
47. Metcalf, D. 2010. The colony-stimulating factors and cancer. Nat Rev Cancer 10:425-434.
48. Gowrishankar, K., Gunatilake, D., Gallagher, S. J., Tiffen, J., Rizos, H., and Hersey, P. 2015. Inducible but not constitutive expression of PD-L1 in human melanoma cells is dependent on activation of NF-kappaB. PLoS One 10:e0123410.
49. Bouillez, A., Rajabi, H., Pitroda, S., Jin, C., Alam, M., Kharbanda, A., Tagde, A., Wong, K., and Kufe, D. 2016. Inhibition of MUC1-C suppresses MYC expression and attenuates malignant growth in KRAS mutant lung adenocarcinomas. Cancer Res 76:1538-1548.
50. Hemmi, H., Takeuchi, O., Kawai, T., Kaisho, T., Sato, S., Sanjo, H., Matsumoto, M., Hoshino, K., Wagner, H., Takeda, K., et al. 2000. A Toll-like receptor recognizes bacterial DNA. Nature 408:740-745.
51. Takeshita, F., Leifer, C. A., Gursel, I., Ishii, K. J., Takeshita, S., Gursel, M., and Klinman, D. M. 2001. Cutting edge: Role of Toll-like receptor 9 in CpG DNA-induced activation of human cells. J Immunol 167:3555-3558.
52. Pivarcsi, A., Muller, A., Hippe, A., Rieker, J., van Lierop, A., Steinhoff, M., Seeliger, S., Kubitza, R., Pippirs, U., Meller, S., et al. 2007. Tumor immune escape by the loss of homeostatic chemokine expression. Proc Natl Acad Sci USA 104:19055-19060.
53. Ronkainen, H., Hirvikoski, P., Kauppila, S., Vuopala, K. S., Paavonen, T. K., Selander, K. S., and Vaarala, M. H. 2011. Absent Toll-like receptor-9 expression predicts poor prognosis in renal cell carcinoma. J Exp Clin Cancer Res 30:84.
55. Akira, S., Takeda, K., and Kaisho, T. 2001. Toll-like receptors: critical proteins linking innate and acquired immunity. Nat Immunol 2:675-680.
56. Schroder, K., Hertzog, P. J., Ravasi, T., and Hume, D. A. 2004. Interferon-gamma: an overview of signals, mechanisms and functions. J Leukoc Biol 75:163-189.
57. Seliger, B., Ruiz-Cabello, F., and Garrido, F. 2008. IFN inducibility of major histocompatibility antigens in tumors. Adv Cancer Res 101:249-276.
58. Agnello, D., Lankford, C. S., Bream, J., Morinobu, A., Gadina, M., O'Shea, J. J., and Frucht, D. M. 2003. Cytokines and transcription factors that regulate T helper cell differentiation: new players and new insights. J Clin Immunol 23:147-161.
59. Hu, X., Chakravarty, S. D., and Ivashkiv, L. B. 2008. Regulation of interferon and Toll-like receptor signaling during macrophage activation by opposing feedforward and feedback inhibition mechanisms. Immunol Rev 226:41-56.
60. Mak, M. P., Tong, P., Diao, L., Cardnell, R. J., Gibbons, D. L., William, W. N., Skoulidis, F., Parra, E. R., Rodriguez-Canales, J., Wistuba, I I, et al. 2016. A Patient-Derived, Pan-Cancer EMT Signature Identifies Global Molecular Alterations and Immune Target Enrichment Following Epithelial-to-Mesenchymal Transition. Clin Cancer Res 22:609-620.
61. Lou, Y., Diao, L., Parra Cuentas, E. R., Denning, W. L., Chen, L., Fan, Y. H., Byers, L. A., Wang, J., Papadimitrakopoulou, V. A., Behrens, C., et al. 2016. Epithelial-mesenchymal transition is associated with a distinct tumor microenvironment including elevation of inflammatory signals and multiple immune checkpoints in lung adenocarcinoma. ClinCancer Res:[Epub ahead of print].
62. Wang, Q., Carroll, J. S., and Brown, M. 2005. Spatial and temporal recruitment of androgen receptor and its coactivators involves chromosomal looping and polymerase tracking. Mol Cell 19:631-642.
63. Tagde, A., Rajabi, H., Bouillez, A., Alam, M., Gali, R., Bailey, S., Tai, Y. T., Hideshima, T., Anderson, K., Avigan, D., et al. 2016. MUC1-C drives MYC in multiple myeloma. Blood 127:2587-2597.
64. Welsh, E. A., Eschrich, S. A., Berglund, A. E., and Fenstermacher, D. A. 2013. Iterative rank-order normalization of gene expression microarray data. BMC Bioinformatics 14:153.
65. Samur, M. K. 2014. RTCGAToolbox: a new tool for exporting TCGA Firehose data. PLoS One 9:e106397.
66. Bouillez, A., Rajabi, H., Jin, C., Samur, M., Tagde, A., Alam, M., Hiraki, M., Maeda, T., Hu, X., Adeegbe, D., et al. 2017. MUC1-C integrates PD-L1 induction with repression of immune effectors in non-small cell lung cancer. Oncogene March 13 [Epub ahead of print].
67. Gao, J., Aksoy, B. A., Dogrusoz, U., Dresdner, G., Gross, B., Sumer, S. O., Sun, Y., Jacobsen, A., Sinha, R., Larsson, E., et al. 2013. Integrative analysis of complex cancer genomics and clinical profiles using the cBioPortal. Sci Signal 6:pl1.
68. Gyorffy, B., Surowiak, P., Budczies, J., and Lanczky, A. 2013. Online survival analysis software to assess the prognostic value of biomarkers using transcriptomic data in non-small-cell lung cancer. PLoS One 8:e82241.
69. Rajabi, H., and Kufe, D. 2017. MUC1-C oncoprotein integrates a program of EMT, epigenetic reprogramming and immune evasion in human carcinomas. BBA Reviews on Cancer 1868:117-122.
70. Kufe, D. 2013. MUC1-C oncoprotein as a target in breast cancer: activation of signaling pathways and therapeutic approaches. Oncogene 32:1073-1081.
71. Rowse, G. J., Tempero, R. M., VanLith, M. L., Hollingsworth, M. A., and Gendler, S. J. 1998. Tolerance and immunity to MUC1 in a human MUC1 transgenic murine model. Cancer Res. 58:315-321.
72. Gong, J., Chen, D., Kashiwaba, M., Li, Y., Takeuchi, H., Qu, H., Rowse, G. J., Gendler, S. J., and Kufe, D. W. 1998. Reversal of tolerance to human MUC1 antigen in MUC1 transgenic mice immunized with fusions of dendritic and carcinoma cells. Proc. Natl. Acad. Sci. USA 95:62179-6283.
73. Agata, N., Kawano, T., Ahmad, R., Raina, D., Kharbanda, S., and Kufe, D. 2008. MUC1 oncoprotein blocks death receptor-mediated apoptosis by inhibiting recruitment of caspase-8. Cancer Res. 68:6136-6144.
74. David, J. M., Hamilton, D. H., and Palena, C. 2016. MUC1 upregulation promotes immune resistance in tumor cells undergoing brachyury-mediated epithelial-mesenchymal transition. Oncoimmunology 5:e1117738.
75. Chen S, Lee L F, Fisher T S, Jessen B, Elliott M, Evering W, et al. Combination of 4-1BB agonist and PD-1 antagonist promotes antitumor effector/memory CD8 T cells in a poorly immunogenic tumor model. Cancer Immunology Research 2015; 3:149-60.
76. Hatzis C, Pusztai L, Valero V, Booser D J, Esserman L, Lluch A, et al. A genomic predictor of response and survival following taxane-anthracycline chemotherapy for invasive breast cancer. JAMA 2011; 305:1873-81.
77. Tagde A, Rajabi H, Bouillez A, Alam M, Gall R, Bailey S, et al. MUC1-C drives MYC in multiple myeloma. Blood 2016; 127:2587-97.
78. Dent R, Trudeau M, Pritchard K I, Hanna W M, Kahn H K, Sawka C A, et al. Triple-negative breast cancer: clinical features and patterns of recurrence. Clin Cancer Res 2007; 13:4429-34.
79. Balko J M, Giltnane J M, Wang K, Schwarz L J, Young C D, Cook R S, et al. Molecular profiling of the residual disease of triple-negative breast cancers after neoadjuvant chemotherapy identifies actionable therapeutic targets. Cancer Discov 2014; 4:232-45.
80. Shah S P, Roth A, Goya R, Oloumi A, Ha G, Zhao Y, et al. The clonal and mutational evolution spectrum of primary triple-negative breast cancers. Nature 2012; 486:395-9.
81. Sikov W M, Berry D A, Perou C M, Singh B, Cirrincione C T, Tolaney S M, et al. Impact of the addition of carboplatin and/or bevacizumab to neoadjuvant once-per-week paclitaxel followed by dose-dense doxorubicin and cyclosphosphamide on pathologic complete response rates in stage II to III triple-negative breast cancer: CALGB 40603 (Alliance). J Clin Oncol 2015; 33:13-21.
82. Adams S, Gray R J, Demaria S, Goldstein L, Perez E A, Shulman L N, et al. Prognostic value of tumor-infiltrating lymphocytes in triple negative breast cancers from two phase III randomized adjuvant breast cancer trials: ECOG 2197 and ECOG 1199. J Clin Oncol. 2014; 32:2959-66.
83. Denkert C, von Minckwitz G, Brase J C, Sinn B V, Gade S, Kronenwett R, et al. Tumor-infiltrating lymphocytes and response to neoadjuvant chemotherapy with or without carboplatin in human epidermal growth factor receptor 2-positive and triple-negative primary breast cancers. J Clin Oncol 2015; 33:983-891.
84. Garcia-Teijido P, Cabal M L, Fernandez I P, Perez Y F. Tumor infiltrating lymphocytes in triple negative breast cancer: The future of immune targeting. Clin Med Insights Oncol 2016; 10:31-9.
85. Groschel S, Bommer M, Hutter B, Budczies J, Bonekamp D, Heining C, et al. Integration of genomics and histology revises diagnosis and enables effective therapy of refractory cancer of unknown primary with PDL1 amplification. Cold Spring Harb Mol Case Stud 2016; 2:a001180.
86. Soliman H, Khalil F, Antonia S. PD-L1 expression is increased in a subset of basal type breast cancer cells. PLoS One 2014; 9:e88557.
87. Beckers R K, Selinger C I, Vilain R, Madore J, Wilmott J S, Harvey K, et al. Programmed death ligand 1 expression in triple-negative breast cancer is associated with tumour-infiltrating lymphocytes and improved outcome. Histopathology 2016; 69:25-34.
88. Guo L, Li W, Zhu X, Ling Y, Qiu T, Dong L, et al. PD-L1 expression and CD274 gene alteration in triple-negative breast cancer: implication for prognostic biomarker. Springerplus 2016; 5:805.
89. Muenst S, Schaerli A R, Gao F, Daster S, Trella E, Droeser R A, et al. Expression of programmed death ligand 1 (PD-L1) is associated with poor prognosis in human breast cancer. Breast Cancer Res Treat 2014; 146:15-24.
90. Sabatier R, Finetti P, Mamessier E, Adelaide J, Chaffanet M, Ali H R, et al. Prognostic and predictive value of PDL1 expression in breast cancer. Oncotarget 2015; 6:5449-64.
91. Ali H R, Glont S E, Blows F M, Provenzano E, Dawson S J, Liu B, et al. PD-L1 protein expression in breast cancer is rare, enriched in basal-like tumours and associated with infiltrating lymphocytes. Ann Oncol 2015; 26:1488-93.
92. Pusztai L, Karn T, Safonov A, Abu-Khalaf M M, Bianchini G. New strategies in breast cancer: Immunotherapy. Clin Cancer Res 2016; 22:2105-10.
93. Nanda R, Chow L Q, Dees E C, Berger R, Gupta S, Geva R, et al. Pembrolizumab in patients with advanced triple-negative breast cancer: Phase Ib KEYNOTE-012 study. J Clin Oncology 2016 May 2; [Epub ahead of print].
94. Alam M, Ahmad R, Rajabi H, Kharbanda A, Kufe D. MUC1-C oncoprotein activates ERK→C/EBPβ-mediated induction of aldehyde dehydrogenase activity in breast cancer cells. J Biol Chem 2013; 288:30829-903.
95. Raina D, Uchida Y, Kharbanda A, Rajabi H, Panchamoorthy G, Jin C, et al. Targeting the MUC1-C oncoprotein downregulates HER2 activation and abrogates trastuzumab resistance in breast cancer cells. Oncogene 2014; 33:3422-31.
96. Rajabi H, Tagde A, Alam M, Bouillez A, Pitroda S, Suzuki Y, et al. DNA methylation by DNMT1 and DNMT3b methyltransferases is driven by the MUC1-C oncoprotein in human carcinoma cells. Oncogene 2016; 35:6439-45.
97. Alam M, Bouillez A, Tagde A, Ahmad R, Rajabi H, Maeda T, et al. MUC1-C represses the Crumbs complex polarity factor CRB3 and downregulates the Hippo pathway. Molecular Cancer Research 2016; 14:1266-76.
98. Hiraki M, Maeda T, Bouillez A, Alam M, Tagde A, Hinohara K, et al. MUC1-C activates BMI1 in human cancer cells. Oncogene 2016; Nov. 28 [Epub ahead of print].
99. Alam M, Rajabi H, Ahmad R, Jin C, Kufe D. Targeting the MUC1-C oncoprotein inhibits self-renewal capacity of breast cancer cells. Oncotarget 2014; 5:2622-34.
100. Takahashi H, Jin C, Rajabi H, Pitroda S, Alam M, Ahmad R, et al. MUC1-C activates the TAK1 inflammatory pathway in colon cancer. Oncogene 2015; 34:5187-97.
101. Kao J, Salari K, Bocanegra M, Choi Y L, Girard L, Gandhi J, et al. Molecular profiling of breast cancer cell lines defines relevant tumor models and provides a resource for cancer gene discovery. PLoS One 2009; 4:e6146.
102. Neve R M, Chin K, Fridlyand J, Yeh J, Baehner F L, Fevr T, et al. A collection of breast cancer cell lines for the study of functionally distinct cancer subtypes. Cancer Cell 2006; 10:515-27.
103. Chavez K J, Garimella S V, Lipkowitz S. Triple negative breast cancer cell lines: one tool in the search for better treatment of triple negative breast cancer. Breast Dis 2010; 32:35-48.
104. Alsuliman A, Colak D, Al-Harazi O, Fitwi H, Tulbah A, Al-Tweigeri T, et al. Bidirectional crosstalk between PD-L1 expression and epithelial to mesenchymal transition: significance in claudin-low breast cancer cells. Mol Cancer 2015; 14:149.
105. Lou Y, Diao L, Parra Cuentas E R, Denning W L, Chen L, Fan Y H, et al. Epithelial-mesenchymal transition is associated with a distinct tumor microenvironment including elevation of inflammatory signals and multiple immune checkpoints in lung adenocarcinoma. Clin Cancer Res 2016: [Epub ahead of print].
106. Noman M Z, Janji B, Abdou A, Hasmim M, Terry S, Tan T Z, et al. The immune checkpoint ligand PD-L1 is upregulated in EMT-activated human breast cancer cells by a mechanism involving ZEB-1 and miR-200. Oncoimmunology 2017; 6:e1263412.
107. Pyzer A R, Stroopinsky D, Rosenblatt J, Anastasiadou E, Rajabi H, Washington A, et al. MUC1 inhibition leads to decrease in PD-L1 levels via up-regulation of miRNAs. Leukemia 2017; [Epub ahead of print].
108. Rajabi H, Hiraki M, Tagde A, Alam M, Bouillez A, Christensen C L, et al. MUC1-C activates EZH2 expression and function in human cancer cells. Sci Rep 2017; July [In press]

1. A method of de-repressing an anti-tumor immune response in a subject having cancer comprising administering to the subject a MUC1 inhibitor, a MYC inhibitor, a TAK1 inhibitor, an NF-κB p65 pathway inhibitor, an IKK inhibitor, or a ZEB1 pathway inhibitor.

2. The method of claim 1, wherein the immune response is an innate immune response or an adaptive immune response.

3. The method of claim 1, further comprising administering to the subject an immunotherapy.

4. A method of increasing the efficacy of an immunotherapy regimen comprising administering to the subject who has received or will receive an immunotherapy a MUC1 inhibitor, a MYC inhibitor, a TAK1 inhibitor, an NF-κB p65 pathway inhibitor, an IKK inhibitor, or a ZEB1 pathway inhibitor.

5. The method of claim 4, wherein the immunotherapy is a therapeutic antibody, a CAR T-cell therapy, a dendritic cell/tumor fusion, or a tumor vaccine.

6. The method of claim 1, wherein the inhibitor is administered in an amount sufficient to decrease tumor PD-L1 transcription and or TLR7 transcription.

7. The method of claim 1, wherein the inhibitor is administered in an amount sufficient to increase TLR9, IFNγ, MCP-1 or GM-CSF expression.

8. The method of claim 1, further comprising administering to the subject checkpoint inhibitor.

9. The method of claim 8, wherein the checkpoint inhibitor is PD-1, PD-L1, PD-L2, CTLA-4, LAG-3, B7-H3, B7-H4, Tim3, BTLA, KIR, A2aR, and/or CD200.

10. A method of augmenting the presentation of tumor associated antigen by a tumor comprising administering to said subject a MUC1 inhibitor, a MYC inhibitor, a TAK1 inhibitor, an NF-κB p65 pathway inhibitor, an IKK inhibitor, or a ZEB1 pathway inhibitor.

11. The method of claim 1, wherein the inhibitor is administered in an amount sufficient to increase the expression of TAP-1, TAP-2, MHC or Tapasin.
